(19) United States
(12) Patent Application Publication
(10) Pub. No.: US 2024/0159740 A1
(43) Pub. Date: May 16, 2024

(54) METHOD FOR DIRECT ANALYSIS OF FUNCTIONAL AVIDITY OF T CELLS

(71) Applicant: Charité - Universitätsmedizin Berlin, Berlin (DE)

(72) Inventors: Andreas Thiel, Berlin (DE); Lucie Loyal, Berlin (DE); Larissa Henze, Berlin (DE); Julian Braun, Berlin (DE)

(73) Assignee: Charité - Universitätsmedizin Berlin, Berlin (DE)

(21) Appl. No.: 18/282,280

(22) PCT Filed: Mar. 18, 2022
(86) PCT No.: PCT/EP2022/057253

§ 371 Date: Sep. 15, 2023

(30) Foreign Application Priority Data
Mar. 19, 2021 (EP) 21163756.6

(51) Int. Cl. G01N 33/50; G01N 33/569

(52) U.S. Cl. CPC G01N 33/5091; G01N 33/56983; G01N 2333/165; G01N 2469/20

(57) ABSTRACT

The invention relates to methods for assessing a level of T cell activation by one or more antigens or for assessing functional avidity of T cells for one or more antigens, the methods comprising providing a cell population comprising T cells, contacting said cell population with one or more antigens in vitro, and determining a level of a T-cell receptor (TCR) complex and/or component thereof in a sub-set of T cells of said cell population. The invention further relates to a kit for performing the method according to the invention

The present invention is in the field of immunology and methods for assessing immune responses.

The invention relates to methods for assessing a level of T cell activation by one or more antigens or for assessing functional avidity of T cells for one or more antigens, the methods comprising providing a cell population comprising T cells, contacting said cell population with one or more antigens in vitro, and determining a level of a T-cell receptor (TCR) complex and/or component thereof in a sub-set of T cells of said cell population. The invention further relates to a kit for performing the method according to the invention.

### BACKGROUND

Immunity to a specific antigen, antigen mixture, or pathogen or pathogen mixtures (whether in experimental models or in humans) is detected and analyzed either by serological analysis (analysis of antibodies present in serum) or by the quantity or functions of specific blood cells or cells that can be isolated from tissues or experimental models. A distinction is made between natural immunity, which is provided by a wide variety of factors and the interaction of often several cell types, and so-called adaptive immunity, in which primarily B and T lymphocytes act as effector cells. Both cell types express specific immune receptors that recognize antigens of various types either in their native form (B cells) or peptide or substance fragments of these antigens in the context of endogenous protein structures, such as the HLA molecules with their so-called immune receptors.

The strength and duration of the interaction of B cell receptors of B cells, and T cell receptors of T cells, with antigen or fragment-HLA complexes determine their development, but more importantly also their initial activation. So far, the main focus on this initial activation has been to understand how costimulatory molecules and soluble factors, such as various cytokines, influence initial differentiation of B and T cells upon activation. Less studied is the impact of duration and strength of immune receptor-antigen interactions on the further functionality of B- or T-cells.

In vitro studies in T cells indicate that T cells activated with lower strength, are typically less fit/effective. For example, it has been shown that T cells that experience short or weak activation signals cannot respond to further signals, such as IL7 or IL15, and are not maintained in in vivo models. Thus, the concept has been proposed that weakly activated T cells may play only a minor role in immunity to pathogens or antigens because they experience clear differentiation disadvantages during the course of initial activation.

However, this is contradicted by the fact that a large heterogeneity can be observed in the interactions of immune receptors of antigen-experienced B and T cells with their corresponding antigens. Mechanisms enabling this heterogeneity must however have evolved evolutionarily to ensure that a highly diverse repertoire of immune receptors is always provided against a wide variety of pathogens, which can rapidly change their antigenic structures by mutation or exchange of antigens.

In the course of the COVID-19 pandemic, it has been illustrated in a wide variety of scientific publications that immune responses against the SARS-CoV-2 virus and its diverse structural and non-structural antigens appear to have a decisive influence on the outcome of an infection. What was known, of course, was that specific immunodominant antigens often must be recognized to overcome infection, as in this case. Surprisingly, significant parts of the acute and also the memory immune response seem to be characterized by the presence of different functional avidities with respect to the interaction of immune receptors with antigenic structures.

The decisive factor is how many immune receptors bind an antigen or an antigen-HLA structure, and with what affinity over what period of time, resulting in immune cell activation. So far, this “quality” of an immune response cannot be analyzed directly. The immune receptors in question must either be characterized “molecularly” and re-expressed for further investigation, or the immune cells in question must be isolated and/or the response to the corresponding antigen analyzed in more detail by means of a titration experiment. A direct and simple determination of a functional avidity has not been previously developed.

In the prior art, techniques are disclosed that comprise culturing cell mixtures for a defined time with antigen, antigen mixtures or even whole pathogens or substance mixtures. Various leukocyte populations, and in particular lymphocytes, express receptors in the resting state with which they can bind certain antigenic structures, either in native form or as fragments. Depending on the strength and duration of the interaction, specific activation of the respective cell generally occurs in a very short time. It has been previously disclosed that in the course of this activation, characteristically for T cells, the CD40L molecule is very rapidly expressed. CD40L can thus be used as a direct signature for the amount of T cells recognizing a specific antigen (or mixtures). However, this procedure cannot be used to determine the functional quality of the cells analyzed.

Although analyses of distinct cytokines can be performed simultaneously, no statement can be made about the strength and duration of the original activation of the cell with the respective ligand. On the one hand, the analysis of co-expressed cytokines and other messengers during activation, especially of experienced leukocyte populations, is very important for the understanding of the function of these cells in physiological and especially also pathophysiological situations. On the other hand, the strength and duration of direct activation also determine whether immune reactions can be completed “successfully”.

Prominent examples of this are malignant diseases and infections. Especially in the current COVID-19 pandemic with SARS-CoV-2 as a new pathogen, many unresolved questions arise where it is of highest interest to characterize qualities of immune responses directly, in detail and precisely. For example, several publications have recently described that certain proportions of T-cell responses against the novel SARS-CoV-2 virus in healthy, previously unexposed individuals are due to cross-reactivities against homologous and also non-homologous antigens that have not been further characterized.

Moreover, such responses have been described to be executed in part by T cells characterized by low functional avidity. So far, there are approaches to analyze such a low functional avidity. One possibility is to titrate the stimulus in question and thereby analyze an indirect measure of functional avidity. Cells with high functional avidity also respond with lower-dosed antigen stimuli.

However, many stimulation approaches are necessary and with limited amounts of e.g. blood samples this is difficult to carry out in practice. In addition, secondary effects arise when antigen mixtures are tested. Especially in T cell assays it can happen that when peptide pools are used, certain peptides with high affinity to HLA molecules occupy them and thus ‘displace’ the actual antigenic peptides, especially in titration experiments. The results can therefore be skewed due to such effects. It is also possible to isolate specific reacting cells in a first step with optimal concentrations of the corresponding antigens and then subsequently titrate the antigen stimulus in a second culture. This then increases the specificity of the analysis, but introduces other difficulties, such as the partially limited reactivability of cells after initial activation for time periods that have not yet been clearly determined.

To circumvent these limitations, it would be of significant practical utility to be able to analyze functional avidity directly, which has not been possible to date.

### SUMMARY THE INVENTION

In light of the prior art the technical problem underlying the present invention is to provide alternative and/or improved means for assessing functional avidity of T cells for one or more antigens and/or assessing a level of T cell activation caused by one or more antigens. The objective of the invention may also be considered the provision of alternative and/or improved means for assessing the strength and/or quality of an immune response in a subject against any given one or more antigens.

This problem is solved by the features of the independent claims. Preferred embodiments of the present invention are provided by dependent claims.

The invention therefore relates to a method for assessing functional avidity of T cells for one or more antigens.

The term functional avidity preferably refers to a measure of a level of T cell activation caused by one or more antigens. The strength of T cell activation can therefore be assessed, preferably quantified, by assessing to what extent a T cell population, preferably a T helper cell population, can be activated by any given one or more antigens. Previous methods had disclosed means for determining the number of activated T cells in a cell population, for example by determining the amount of CD40L+ T cells (U.S. Pat. No. 7,659,084B2). However, until the present time, no means existed to determine a level of T cell activation, i.e. a preferably quantitative measure of the strength of an immune response, preferably determined through the level of T cell activation after incubation with any one or more antigens.

The invention therefore relates to a method for assessing a level of T cell activation caused by one or more antigens, comprising:
providing a cell population comprising T cells,
contacting said cell population with one or more antigens in vitro, and
determining a level of a T-cell receptor (TCR) complex and/or component thereof in a sub-set of T cells of said cell population.

The invention also relates to a method for assessing functional avidity of T cells for one or more antigens, comprising:
providing a cell population comprising T cells,
contacting said cell population with one or more antigens in vitro, and
determining a level of a T-cell receptor (TCR) complex and/or component thereof in a sub-set of T cells of said cell population.

Additional embodiments relate to those presented in the claims and in the following disclosure.

One of the surprising benefits of the methods according to the invention is that they can be employed to determine the immunocompetence of a patient to a certain antigen, such as a pathogen or infection. In embodiments described herein, the methods and kits according to the invention provide improved means for analyzing the immune competence and/or level of T-cell activation and/or functional avidity of T-cells of a patient to an infection with a certain pathogen and/or for determining a specific pathogen infection in a patient. Examples of the beneficial effects of the different aspects of the present invention are shown in the Examples and Figures below.

In embodiments, the cell population comprising a T cell comprises or consists of, or is derived from, a bodily fluid from a subject, such as blood or part thereof or cerebrospinal fluid, preferably from a human subject, or an in vitro cell population of T cells, for example a population of T cells isolated from a subject and cultivated in vitro, or a population of T cells generated in vitro, wherein most preferably the cell population comprising a T cell comprises or consists of peripheral blood mononuclear cells (PBMC).

In embodiments of the methods according to the invention, the bodily fluid from a subject is blood or part thereof or cerebrospinal fluid.

In embodiments of the methods according to the invention, the cell population comprising a T cell comprises or consists of, or is derived from a bodily fluid from a human subject, or an in vitro cell population of T cells, for example a population of T cells isolated from a subject and cultivated in vitro, or a population of T cells generated in vitro.

In embodiments of the methods according to the invention, the sub-set of cells in which a T-cell receptor (TCR) and/or component thereof is determined comprises or consists of T helper cells, preferably CD4+ T cells, more preferably activated T helper cells, more preferably CD40L+, 4-1BB+ and/or CD4+ T cells.

In embodiments of the methods according to the invention, the sub-set of cells in which a T-cell receptor (TCR) and/or component thereof is determined comprises or consists of CD40L+, 4-1BB+, CD4+ and/or CD8+ T cells.

In embodiments of the methods according to the invention, the sub-set of cells in which a T-cell receptor (TCR) and/or component thereof is determined comprises or consists of CD4+ T cells.

In embodiments of the methods according to the invention, the sub-set of cells in which a T-cell receptor (TCR) and/or component thereof is determined comprises or consists of CD8+ T cells.

In embodiments of the methods according to the invention, the sub-set of cells in which a T-cell receptor (TCR) and/or component thereof is determined comprises or consists of CD69+ and/or OX40+ T cells.

In the context of the present invention, embodiments are not limited to helper T cells. Also, CD8+ T cells can be analyzed be as well as those T cells expressing both CD4 and CD8 markers or none of them. In preferred embodiments the sub-set of cells comprises a T-cell receptor (TCR) and/or component thereof on their surface.

Hence, in embodiments of the methods according to the invention, the sub-set of cells in which a T-cell receptor (TCR) and/or component thereof is determined comprises or consists of activated T helper cells.

In embodiments of the methods according to the invention, the sub-set of cells in which a T-cell receptor (TCR) and/or component thereof is determined comprises or consists of any given T cell population that presents a TCR on the surface of said cells.

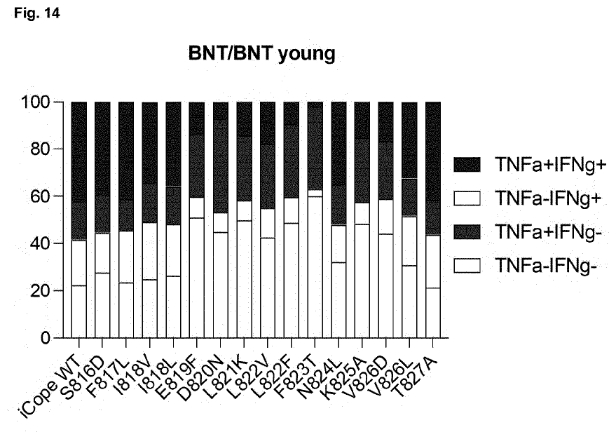

In embodiments of the invention CD40L+ and 41 BB+ are two possible T-cell activation markers that can be analyzed. In embodiments of the invention further T-cell activation markers such as CD69, OX40 and/or cytokines, such as e.g. IFNg, IL-2, or TNFα, can be analyzed alternatively or in addition to the afore mentioned (surface) markers (see e.g. Example 3 and FIG. 14).

In embodiments, the T-cell activation marker may be selected from any one or more markers that is expressed on the surface of a T cell, such as a surface receptor, such as but not limited to CD25 (the IL-2 receptor), CD71 (the transferrin receptor), or co-stimulatory molecules, such as, without limitation, CD26, CD27, CD28, CD30, CD154 or CD40L, and/or CD134.

In embodiments of the methods according to the invention, determining a level of a T-cell receptor (TCR) complex and/or component thereof comprises or consists of determining a level of TCR alpha (α), beta (β), gamma (γ) or delta (δ) chains, preferably constant regions thereof, or cluster of differentiation 3 (CD3), preferably wherein a level of CD3 is determined.

In aspects and embodiments of the invention described herein, cell mixtures are cultured in vitro with the respective ligands, antigens, antigen mixtures, pathogens and/or substance mixtures to be investigated, and then a reduction in expression of components of (molecules comprised in) the T cell receptor (TCR) complex is analyzed.

In some embodiments, antigen-reactive cells are first identified by activation-induced CD40L expression (or comparable other activation markers) and then, within the activated cells, the expression of CD3 or other molecules of the TCR complex (a TCR component) is analyzed.

In embodiments of the methods according to the invention, determining a level of a T-cell receptor (TCR) complex and/or component thereof comprises or consists of determining a level of CD3.

The proportion of cells with reduced (low levels or downregulated) amounts of a TCR component, preferably CD3, indicate (or determine) a level of functional avidity.

The functional avidity relates in some embodiments to a measure of the strength of binding between the antigen and TCR.

The functional avidity relates in some embodiments to a measure of effectiveness of a T-cell in inducing and/or supporting an immune response induced by the antigen.

The functional avidity relates in some embodiments to a measure of immune response strength, said immune response mediated by CD4+ T helper cells.

The functional avidity relates in some embodiments to a measure of immune response strength, capacity, speed and/or other quality of an immune response, said immune response mediated by the adaptive immune response.

The functional avidity relates in some embodiments to a measure of immune response strength, in particular a measure of CD4+ T cell antigen-specific activation.

The stronger and longer the interaction of immune receptor and ligand (antigen), the stronger this downregulation of TCR component (eg CD3), and the greater the avidity of the TCR-antigen interaction, indicating a greater effectiveness in an immune response after binding between the TCR and antigen.

In embodiments, the methods according to the invention comprise determining the proportion of cells in said sub-set of cells (preferably CD40L+, 4-1BB+, CD4+ T cells) in which the level of a T-cell receptor (TCR) complex and/or component thereof (preferably CD3) is below a first threshold value (TCR^{low} cells, preferably CD3^{low} cells).

In embodiments of the methods according to the invention, the proportion of CD40L+, 4-1BB+, CD4+ T cells is determined in said sub-set of cells.

In embodiments the methods according to the invention, comprise determining the proportion of cells in said sub-set of cells in which the level of a T-cell receptor (TCR) complex and/or CD3-component thereof is below a first threshold value (TCR^{low} cells).

In embodiments the methods according to the invention, comprise determining the proportion of cells in said sub-set of cells in which the level of a T-cell receptor (TCR) complex and/or component thereof is below a first threshold value (TCR^{low}-CD3^{low} cells).

In embodiments of the methods according to the invention, the proportion of TCR^{low} (CD3^{low}) cells in said sub-set of cells indicates a level of T cell functional avidity and/or activation.

In embodiments of the methods according to the invention, the proportion of TCRlow (CD3^{low}) cells in said sub-set of cells is positively correlated with the level of T cell functional avidity and/or activation.

In embodiments of the methods according to the invention, determining the level of a T-cell receptor (TCR) complex and/or component thereof (preferably CD3) in a sub-set of T cells of said cell population is carried out using flow cytometry of antibody labelled cells (preferably wherein the sub-set of cells is labelled using CD40L, 4-1BB, CD4 and CD3 antibodies).

In embodiments of the methods according to the invention, determining the level of a T-cell receptor (TCR) complex and/or component thereof (preferably CD3) in a sub-set of T cells of said cell population is carried out using flow cytometry of antibody labelled cells (preferably wherein the sub-set of cells is labelled using anti-CD40L, 4-1BB, CD4, CD3, CD69, OX40 and/or IFNg, IL-2, or TNFα antibodies).

In embodiments of the methods according to the invention, determining the level of a T-cell receptor (TCR) complex and/or CD3-component thereof in a sub-set of T cells of said cell population is carried out using flow cytometry of antibody labelled cells.

In embodiments of the methods according to the invention, determining the level of a T-cell receptor (TCR) complex and/or component thereof in a sub-set of T cells of said cell population is carried out using flow cytometry of antibody labelled cells, wherein the sub-set of cells is labelled using CD40L, 4-1BB, CD4 and CD3 antibodies.

In embodiments of the method for assessing a level of T cell activation caused by one or more antigens, contacting said cell population with one or more antigens in vitro comprises cultivation of said cells with said one or more antigens for 6 to 36 hours, preferably 10 to 24 hours, more preferably 12 to 20 hours, more preferably about 16 hours. In some embodiments the duration of the cultivation of said cells with said one or more antigens is between 2 to 36 hours.

In embodiments of the methods according to the invention, contacting said cell population with one or more antigens in vitro comprises cultivation of said cells with said one or more antigens for 10 to 24 hours.

In embodiments of the methods according to the invention, contacting said cell population with one or more antigens in vitro comprises cultivation of said cells with said one or more antigens for preferably 12 to 20 hours.

In embodiments of the methods according to the invention, contacting said cell population with one or more antigens in vitro comprises cultivation of said cells with said one or more antigens for about 16 hours. In some embodiments the cultivation for about 16 h represents the optimum for the combination of the activation markers 41 BB and CD40L.

In other embodiments the incubation optimum may be between 6 h or 12 h or between 2 and 36 hours of cultivation. In embodiments the cultivation optimum depends on the surface markers and/or cytokines analyzed.

In embodiments of the methods according to the invention, comprises additionally determining a level of one more cytokines after contacting said cell population with one or more antigens in vitro.

In some embodiments such cytokines may comprise IFNg, IL-2 and/or TNFα.

In embodiments of the methods according to the invention, said one or more antigens comprises or consists of one or more ligands, antigens, pathogens and/or mixtures or fragments thereof.

In embodiments of the methods according to the invention, the method is employed to assess the strength and/or quality of an immune response in a subject, from which said cell population comprising T cells was obtained, against one or more antigens of interest.

In embodiments of the methods according to the invention, the one or more antigens is of pathological relevance, preferably wherein the one or more antigens comprise or consist of an autoantigen, a tumor antigen, a pathogen, or antigenic part or mixture thereof.

In embodiments of the methods according to the invention, the one or more antigens is of pathological relevance and wherein the one or more antigens comprise or consist of an autoantigen, a tumor antigen, a pathogen, or antigenic part or mixture thereof.

In embodiments of the methods according to the invention, the method is employed to monitor the status of a disease and/or condition of a subject with a persistent immune-related medical condition, such as a viral infection, such as HCV, HIV infection, or an autoimmune disease.

In embodiments of the methods according to the invention, the one or more antigens is, comprises and/or is derived from SARS-CoV, preferably SARS-CoV-2, more preferably SARS-CoV-2 spike glycoprotein, more preferably SARS-CoV-2 spike glycoprotein S-II, more preferably a peptide as described herein, for example as described herein, wherein preferably the method is employed to assess (preferably prognose) an immune response of said subject to SARS-CoV-2 (i.e. to assess the strength of an immune response against a SARS-CoV-2 infection), or to assess a risk of a subject in developing a severe acute respiratory syndrome (SARS) or other adverse event or severe medical condition associated with a SARS-CoV (preferably SARS-CoV-2) infection.

The analyses can, in some embodiments, be performed in parallel with analyses on the differentiation status or functional cytokine expression pattern of cells in a sample.

The analysis of the present invention allows direct characterization of the functional avidity of reactive cells, such as T cells.

The analysis as described herein can be performed directly. No titration of ligands or antigens is necessary and no isolation and recultivation of reactive cells is required.

In some embodiments, the assay can be performed in a period of 2-24 h after activation. In some embodiments, activation refers to initiation of culturing of cells with an antigen of interest.

One embodiment relates to assessment of TCR component downregulation, such as CD3 levels, after 2 hours, or at 10-24 h, or 12-20 h, preferably 16 h after activation, as this is an optimal time period for the selection of activation markers, during which additional analyses (e.g. cytokine production) can also be performed.

The following examples are presented to describe particular embodiments of the invention, without being limiting in scope.

Various applications are conceivable in which, for the first time, critical conclusions can be drawn about the quality of immune responses in malignancies, in infections, in autoimmune diseases, and in the status of and/or recovery from various disease states. Immune responses can be characterized not only functionally in terms of distinct messengers or activation markers, but also in terms of their functional avidity. Poor functional avidity will generally be associated with a non-efficient (because, for example, too slow) immune response.

The method according to the invention can be a strong tool in tumor therapy. In embodiments the high avidity T cells against tumor antigens can be identified ex vivo and expanded in vitro, optionally followed by adoptive transfer into the patient (wherein all steps are donor specific).

One application of such embodiments would be a variant CAR (Chimeric Antigen Receptor) T cell therapy. Moreover, embodiments of the method according to the invention can be used to identify high avidity T cells against tumor antigens in a HLA dependent manner. Therein the TCR can be transduced into T cells of tumor patients followed by adoptive transfer. In these embodiments universal, MHC dependent tumor-targeting T cells are generated, which in specific cases might potentially work for any donor with matching HLA (although potential undesired autoimmune reaction based on the other HLA should preferably be excluded beforehand).

Accordingly, the methods herein can be applied for the identification of tumor targeting cells for adoptive tumor therapy. In addition, the present methods may be employed for the identification of tumor specific TCR with defined avidity (characteristic for high avidity=CD³ₗₒ, for low avidity=CD³ₕᵢ).

In the case of infections, this means that the pathogen may multiply more rapidly (as in e.g. SARS CoV 2) and then infect cells or tissues systemically. Thus, severe damage occurs. In malignancies, the immune system is likely to efficiently reject tumors only with highly functionally avidities. In autoimmune disease, predictive tests could be developed, or the type or strength of immunosuppression could be adapted to the functional avidity of autoimmunity. A further field of application for the present methods may be related to immunocompromised patients fighting with chronic infections.

A further objective of the invention was to provide alternative and/or improved means for assessing the strength and/or quality of an immune response in a subject against a SARS-CoV infection, or for assessing risk of a subject in developing a severe acute respiratory syndrome (SARS) or other severe medical condition associated with SARS-CoV infection.

The method as described herein can be used to assess an immune response in a subject against a SARS-CoV infection.

In embodiments, the invention relates to a method as described herein in which the one or more antigens is a peptide of up to 25 amino acids comprising or consisting of an amino acid sequence FIEDLLFNKVT (SEQ ID NO 1) or a sequence of at least 80%, preferably at least 90%, sequence identity thereto, preferably of 15 to 25 amino acids, more preferably comprising IEDLLFNKV (SEQ ID NO 3), EDLLFNKVT (SEQ ID NO 4) or FIEDLLFNKVT (SEQ ID NO 1) and optionally 1-4 additional amino acid at the N and/or C-termini.

The inventors identified an immunodominant Coronavirus peptide epitope (iCope) located within the fusion domain of spike recognized by CD4+ T cells in 20% of unexposed individuals, 50-60% of SARS-CoV-2 convalescents and 97% of BNT162b2 vaccinated individuals. iCope and spike-cross-reactive T cells were recruited into primary SARS-CoV-2 infection immune responses and also profoundly into BNT162b2 COVID-19 mRNA vaccination responses. Furthermore, cross-reactive immunity exhibited secondary response kinetics upon primary vaccination and abundance of pre-existing cross-reactive T cells correlated with high functional avidity already early in the immune response as well as with the early induction and stabilization of S1 IgG antibody titers. The invention therefore demonstrates a role of cross-reactive CD4+ T cells in SARS-CoV-2 infection and vaccination, which is relevant in implementing both single-dose vaccination of healthy adults and two-dose vaccination of the elderly.

In embodiments, the (preferably isolated) peptide comprises or consists of an amino acid sequence FIEDLLFNKVT (SEQ ID NO 1) or a sequence of at least 80%, preferably at least 90%, sequence identity thereto, preferably of 15-25 amino acids, more preferably consisting of the amino acid sequence SFIEDLLFNKVTLAD (SEQ ID NO 2).

In embodiments, the (preferably isolated) peptide comprises or consists of an amino acid sequence FIEDLLFNKVT (SEQ ID NO 1) or a sequence of at least 70% sequence identity thereto.

In embodiments, the (preferably isolated) peptide comprises or consists of an amino acid sequence FIEDLLFNKVT (SEQ ID NO 1) or a sequence of at least 80%, preferably at least 90%, sequence identity thereto, preferably consisting of 15 to 25 amino acids, even more preferably of 15 to 20 amino acids.

In embodiments, the (preferably isolated) peptide comprises IEDLLFNKV (SEQ ID NO 3) or EDLLFNKVT (SEQ ID NO 4).

In embodiments, the (preferably isolated) peptide comprises IEDLLFNKV (SEQ ID NO 3) or EDLLFNKVT (SEQ ID NO 4) and at least 1 additional amino acid at the N and/or C-termini.

In embodiments, the (preferably isolated) peptide comprises FIEDLLFNKVT (SEQ ID NO 1) and at least 1 additional amino acid at the N and/or C-termini.

In embodiments, the (preferably isolated) peptide comprises IEDLLFNKV (SEQ ID NO 3), EDLLFNKVT (SEQ ID NO 4) or FIEDLLFNKVT (SEQ ID NO 1) and at least 1-4 additional amino acid at the N and/or C-termini.

In embodiments, the (preferably isolated) peptide comprises or consists of an amino acid sequence FIEDLLFNKVT (SEQ ID NO 1), preferably of at least 11 amino acids, of at least 12 amino acids, of at least 13 amino acids, of at least 14 amino acids, of at least 15 amino acids, of at least 16 amino acids, of at least 17 amino acids, of at least 18 amino acids, of at least 19 amino acids, of at least 20 amino acids, of at least 21 amino acids, of at least 22 amino acids, of at least 23 amino acids, of at least 24 amino acids, or of at least 25 amino acids, of at least 30 amino acids, of at least 40 amino acids, of at least 50 amino acids, of at least 100 amino acids.

In embodiments, the (preferably isolated) peptide comprises or consists of an amino acid sequence FIEDLLFNKVT (SEQ ID NO 1), preferably of 11-200 amino acids, 11-100 amino acids, of 11-50 amino acids, of 12-200 amino acids, of 12-150 amino acids, of 12-100 amino acids, of 12-50 amino acids, of 15-200 amino acids, of 15-100 amino acids, of 15-50 amino acids, of 20-200 amino acids, 20-100 amino acids, 20-50 amino acids, of 11-150 amino acids, of 11-100 amino acids, of 11-50 amino acids, of 11-40 amino acids, of 11-30 amino acids, of 11-25 amino acids, of 11-20 amino acids, of 11-15 amino acids.

In embodiments, the peptide employed in the method comprises or consists of an amino acid sequence selected from the group consisting of:
a) an amino acid sequence comprising or consisting of an amino acid sequence FIEDLLFNKVT (SEQ ID NO 1); wherein the peptide is preferably no longer than 200, preferably no longer than 100 amino acids, preferably no longer than 50 amino acids, more preferably no longer than 25 amino acids;
b) an amino acid sequence comprising or consisting of an amino acid sequence FIEDLLFNKVT (SEQ ID NO 1), wherein the length of the amino acid molecule is 11 to 200 amino acids, preferably 11 to 100 amino acids, preferably 11 to 50 amino acids, more preferably 11 to 25 amino acids, or wherein the sequence comprises one of FIEDLLFNKVT (SEQ ID NO 1), FIEDLLFNKV (SEQ ID NO 6), IEDLLFNKVT (SEQ ID NO 7), IEDLLFNKV (SEQ ID NO 3), or EDLLFNKVT (SEQ ID NO 4), and additional flanking sequences, preferably according to the corresponding sequences of a SARS-CoV spike protein;
c) an amino acid sequence having sufficient sequence identity to be functionally analogous/equivalent to an amino acid sequence according to a) or b), comprising preferably a sequence identity to an amino acid sequence according to a) or b) of at least 70%, 75%, 80%, 85%, preferably 90%, more preferably 95%; and
d) an amino acid sequence of a), b) or c) which is modified by deletions, additions, substitutions, translocations, inversions and/or insertions and is functionally analogous/equivalent to an amino acid sequence according to a), b) or c).

In embodiments, the (preferably isolated) peptide comprises or consists of an amino acid sequence FIEDLLFNKVT (SEQ ID NO 1) and at least 1 additional amino acid at the N and/or C-termini, preferably 1-4 additional amino acids at the N and C-termini.

In some embodiments of the invention, the peptide, preferably according to sequences disclosed herein, may comprise a 0 to 10 amino acid addition or deletion at the N and/or C terminus of a sequence.

In embodiments, the (preferably isolated) peptide comprises or consists of an amino acid sequence FIEDLLFNKVT (SEQ ID NO 1) and at least the amino acids LAD or LADA (SEQ ID NO 5) at the C terminus and/or at least the amino acids SKRS (SEQ ID NO 8) at the N-terminus.

In embodiments, the (preferably isolated) peptide is selected from the group consisting of SFIEDLLFNKVTLAD (SEQ ID NO 2), SKRSFIEDLLFNKVT (SEQ ID NO 9) and FIEDLLFNKVTLADA (SEQ ID NO 10) or a sequence of at least 80%, preferably at least 90%, sequence identity thereto.

In embodiments, the peptide consists of SFIEDLLFNKVTLAD (SEQ ID NO 2).

In embodiments, the peptide of the invention comprises one or more of SEQ ID NO 1-4, 6-7, 9-10. In embodiments, the total length of the peptide is limited, as described herein, for example to a peptide of up to 25 amino acids. Any of the sequences of SEQ ID NO 1-4, 6-7, 9-10 may be considered as a “core” iCOPE peptide sequence. In embodiments, the peptide of the invention comprises at the N and/or C terminus an additional flanking sequence, i.e. further amino acid sequence adjacent to the “core” sequence. In embodiments, the flanking sequence may be one or more of the specific sequences disclosed herein, for example according to SEQ ID NO 5 and/or 8. In embodiments, the sequence flanking the core sequence may be according to the corresponding sequence of a SARS-CoV spike protein. For example, the peptide may comprise a “core” sequence, and additional amino acids derived from one or more SARS-CoV-2 spike proteins, such as those described in SEQ ID NO 11-15, or a sequence of at least 70%, 80%, 90% or 95% sequence identity thereto, or any other spike sequence available to a skilled person.

For example, in the context of the original “Wuhan sequence” of SEQ ID NO 11, the sequence of SEQ ID NO 1 is position within SEQ ID NO 11. Therefore, the peptide of the invention may comprise up to 25 amino acids, comprising SEQ ID NO 1 and the corresponding flanking sequence from SEQ ID NO 11, or any other sequence of SEQ ID NO 11-15. In the context of SEQ ID NO 11, by way of example, the sequence may therefore comprise or consist of a 25 amino acid sequence of SKPSKRSFIEDLLFNKVTLADAGFI (SEQ ID NO 16), wherein SEQ ID NO 1 is represented in bold type.

In further embodiments, the iCOPE peptide, or core peptide, according to the peptide as described herein, may comprise or consist of SEQ ID NO 16, 17, 18, 20 or 21.

The peptide sequences according to the invention represent immunodominant coronavirus peptide (iCope) sequences located within the fusion peptide domain (amino acids 816-830) of the spike protein of SARS-CoV-2. Said peptides have been shown by the inventors to dominate the cross-reactive T cell response against SARS-CoV-2, but are also highly conserved in endemic, human common cold coronaviruses. Accordingly, the peptides of the invention achieve surprising beneficial effects as they can be employed in different SARS-CoV-2-related assays and or even as targets or epitopes for antibody or immune cell responses.

The invention further relates to using a nucleic acid molecule encoding the peptide as described herein. The uses proposed for the peptide are also contemplated for the corresponding coding nucleic acid, and vice versa, and the disclosure of the invention is to be considered accordingly.

In embodiments the invention relates to the an in vitro method for assessing the risk of a subject in developing a severe acute respiratory syndrome (SARS) or other adverse event or severe medical condition associated with SARS-CoV infection (preferably with SARS-CoV-2), comprising use of the peptide according to the invention.

The invention further relates to an in vitro method for assessing the risk of a subject in developing a severe acute respiratory syndrome (SARS) or other adverse event or severe medical condition associated with SARS-CoV infection (preferably with SARS-CoV-2), comprising use of the peptide described herein.

The invention further relates to an in vitro method for assessing the strength and/or quality of an immune response in a subject against a SARS-CoV infection, comprising use of the peptide described herein.

In embodiments the in vitro method for assessing the strength and/or quality of an immune response in a subject against a SARS-CoV infection relates to or comprises a method for assessing a level of T cell activation caused by one or more antigens as described herein, comprising:
providing a cell population comprising T cells,
contacting said cell population with one or more antigens in vitro,
determining a level of a T-cell receptor (TCR) complex and/or component thereof in a sub-set of T cells of said cell population.

In embodiments the in vitro method for assessing the strength and/or quality of an immune response in a subject against a SARS-CoV infection relates to or comprises a method for assessing functional avidity of T cells for one or more antigens as described herein, comprising:
providing a cell population comprising T cells,
contacting said cell population with one or more antigens in vitro, and
determining a level of a T-cell receptor (TCR) complex and/or component thereof in a sub-set of T cells of said cell population.

In embodiments of the in vitro method according to the invention the method comprises a method for assessing a level of T cell activation by one or more antigens, wherein the one or more antigens is, comprises and/or is derived from SARS-CoV, preferably SARS-CoV-2, more preferably SARS-CoV-2 spike glycoprotein, more preferably SARS-CoV-2 spike glycoprotein S-II, more preferably a peptide as described herein, for example as described herein, wherein preferably the method is employed to assess (preferably prognose) an immune response of said subject to SARS-CoV-2 (i.e. to assess the strength of an immune response against a SARS-CoV-2 infection), or to assess a risk of a subject in developing a severe acute respiratory syndrome (SARS) or other adverse event or severe medical condition associated with a SARS-CoV (preferably SARS-CoV-2) infection.

The invention further relates to an in vitro method for assessing and/or selecting a subject for vaccination, for example for selecting a subject for a single vaccination, with a SARS-CoV-2 vaccine, comprising use of the peptide described herein.

The invention further relates to an in vitro method for assessing and/or selecting a subject for vaccination, comprising providing a sample from the subject, and determining the presence and/or absence and/or an amount of one or more antibodies and/or T cells in the sample of said subject that bind the peptide described herein.

In embodiments of the in vitro methods according to the invention the subject has not yet been infected by a SARS-CoV, preferably SARS-CoV-2.

In embodiments the in vitro methods according to the invention comprise any embodiment of a method for assessing a level of T cell activation by one or more antigens as described herein, preferably wherein the peptide according to the invention is employed as the one or more antigens in said method.

While evidence for pre-existing SARS-CoV-2-cross-reactive CD4+ T cells in unexposed individuals is increasing, their functional significance remains unclear. As described below, we comprehensively determined SARS-CoV-2-cross-reactivity and HCoV-reactivity in unexposed individuals. SARS-CoV-2-cross-reactive CD4⁺ T cells were ubiquitous, but their presence decreased with age. Within the spike glycoprotein fusion domain, we identified a universal immunodominant coronavirus-specific peptide epitope (iCope). Pre-existing spike- and iCope-reactive memory T cells were efficiently recruited into mild SARS-CoV-2 infections and their abundance correlated with higher IgG titers. Importantly, the cells were also reactivated after primary BNT162b2 COVID-19 mRNA vaccination in which their kinetics resembled that of secondary immune responses. Our results highlight the functional importance of pre-existing spike-cross-reactive T cells in SARS-CoV-2 infection and vaccination. Abundant spike-specific cross-immunity may be responsible for the unexpectedly high efficacy of current vaccines even with single doses and the high rate of asymptomatic/mild infection courses.

In embodiments of the present invention the one or more antigens are attached to a solid phase. In embodiments the binding or attachment of the one or more antigens to the solid phase can be effected via a spacer. All those chemical compounds having suitable structural and functional preconditions for spacer function can be used as spacers as long as they do not modify or adversely affect the binding behavior or binding abilities of the one or more antigens to a binding partner, such as an antibody, a T-cell, a cellular receptor or any other binding or interaction partner.

In embodiments the binding or attachment of the one or more antigens to the solid phase can be effected via a linker.

In another embodiment of the invention the one or more antigens are used as a soluble or solid phase-bound antigen for direct or indirect detection by an antibody or a cell, preferably a T cell, for example in a sample, preferably in a patient sample.

In another embodiment of the invention the one or more antigens are immobilized. More specifically, the solid phase-bound one or more antigens are bound to organic, inorganic, synthetic and/or mixed polymers, preferably agarose, cellulose, silica gel, polyamides and/or polyvinyl alcohols. In the meaning of the invention, immobilization is understood to involve various methods and techniques to fix the peptides on specific carriers, e.g. according to WO 99/56126 or WO 02/26292. For example, immobilization can serve to stabilize the one or more antigens so that their activity would not be reduced or adversely modified by biological, chemical or physical exposure, especially during storage or in single-batch use. Immobilization of the one or more antigens may in embodiments allow repeated use under technical or clinical routine conditions; furthermore in embodiments, a sample—preferably a patient sample—can be reacted with the one or more antigens in a continuous fashion. In particular, this can be achieved by means of various immobilization techniques, with binding of the one or more antigens to a carrier proceeding in such a way that the three-dimensional structure—particularly in the active center mediating the interaction with cells, preferably T cells, and/or cellular receptors—of the corresponding one or more antigens, would not be changed. In preferred embodiments there is no loss in specificity to the cells, preferably T cells, and/or cellular receptors of patients as a result of such immobilization. In the meaning of embodiments of the invention, at least three different methods can be used for immobilization:
(i) Crosslinking: in crosslinking, the one or more antigens are fixed to one another without adversely affecting their activity. Advantageously, they are no longer soluble as a result of such crosslinking.
(ii) Binding to a carrier: binding to a carrier proceeds via adsorption, ionic binding or covalent binding, for example. Such binding may also take place inside microbial cells or liposomes or other membranous, closed or open structures. Advantageously, the one or more antigens are not adversely affected by such fixing. For example, multiple or continuous use of carrier-bound one or more antigens is possible with advantage in clinical diagnosis or therapy.
(iii) Inclusion: inclusion in the meaning of the invention especially proceeds in a semipermeable membrane in the form of gels, fibrils or fibers. Advantageously, encapsulated antigens are separated from the surrounding sample solution by a semipermeable membrane in such a way that interaction with the cells, preferably T cells, and/or cellular receptors is still possible. Various methods are available for immobilization, such as adsorption on an inert or electrically charged inorganic or organic carrier. For example, such carriers can be porous gels, aluminum oxide, bentonite, agarose, starch, nylon or polyacrylamide. Immobilization proceeds via physical binding forces, frequently involving hydrophobic interactions and ionic binding. Advantageously, such methods are easy to handle and have little influence on the conformation of the one or more antigens.

In embodiments another method may be covalent binding to carrier materials. In addition, the carriers may have reactive groups forming homopolar bonds with amino acid side chains of antigens. Suitable groups in antigens are carboxy, hydroxy and sulfide groups and especially amino groups. Aromatic groups offer the possibility of diazo coupling. The surface of microscopic porous glass particles can be activated by treatment with silanes and subsequently reacted with the one or more antigens. For example, hydroxy groups of natural polymers can be activated with bromocyanogen and subsequently coupled with the antigens. Advantageously, a large number of antigens may undergo direct covalent binding with polyacrylamide resins. Inclusion in three-dimensional networks involves inclusion of the peptides in ionotropic gels or other structures well-known to those skilled in the art. More specifically, the pores of the matrix are such in nature that the peptides are retained, allowing interaction with the target molecules. In crosslinking, the peptides are converted into polymer aggregates by crosslinking with bifunctional agents. Such structures are gelatinous, easily deformable and, in particular, suitable for use in various reac-tors. By adding other inactive components such as gelatin in crosslinking, advantageous improvement of mechanical and binding properties is possible. In microencapsulation, the reaction volume of the antigens is restricted by means of membranes. For example, microencapsulation can be carried out in the form of an interfacial polymerization. Owing to the immobilization during microencapsulation, the antigens are made insoluble and thus reusable. In the meaning of embodiments of the invention, immobilized antigens are all antigens being in a condition that allows reuse thereof. Restricting the mobility and solubility of the antigens by chemical, biological or physical means advantageously results in lower process cost, particularly when analyzing the functional avidity of T cells in patient samples.

The invention also relates to a diagnostic kit for the determination of the functional avidity of T cells and/or activity of T cells towards a specific antigen using said one or more antigens. The diagnostic kit optionally includes instructions concerning combining the contents of the kit and/or providing a formulation for the detection of the functional avidity of T cells and/or activity of T cells towards a specific antigen. For example, the instruction can be in the form of an instruction leaflet or other medium providing the user with information as to the type of method wherein the substances mentioned are to be used. Obviously, the information need not necessarily be in the form of an instruction leaflet, and the information may also be imparted via the Internet, for example.

The invention further relates to a kit for assessing the functional avidity of T cells for one or more antigens, comprising:
means for determining a level of a T-cell receptor (TCR) complex and/or component thereof in a sub-set of T cells of a cell population, preferably comprising one or more antibodies, more preferably comprising one or more labelled antibodies, wherein the means preferably detect CD3, and
one or more antigens of pathological relevance,
and optionally, means for providing, maintaining and/or culturing a cell population comprising T cells and contacting said cell population with said one or more antigens in vitro, and
optionally, means for determining a level of one more cytokines, preferably one or more antibodies.

The present invention further relates to a kit for assessing the strength and/or quality of an immune response in a subject against a SARS-CoV infection.

In embodiments the invention relates to a kit for performing the method described herein for assessing a level of T cell activation caused by one or more antigens.

In embodiments the invention relates to a kit for performing the method described herein for assessing the functional avidity of T cells for one or more antigens.

In embodiments the invention relates to a kit for assessing a level of T cell activation by one or more antigens, comprising:
means for determining a level of a T-cell receptor (TCR) complex and/or component thereof in a sub-set of T cells of a cell population, preferably comprising one or more antibodies, more preferably comprising one or more labelled antibodies, and/or
optional means for determining a level of one more cytokines, preferably one or more antibodies,
optional one or more antigens, and/or
optional a nucleic acid encoding said one or more antigens, and/or
optional the solid phase according to the invention.

In embodiments the invention relates to a kit for performing the method described herein for assessing the functional avidity of T cells for one or more antigens, comprising:
means for determining a level of a T-cell receptor (TCR) complex and/or component thereof in a sub-set of T cells of a cell population, preferably comprising one or more antibodies, more preferably comprising one or more labelled antibodies, and/or
optional means for determining a level of one more cytokines, preferably one or more antibodies,
optional the one or more antigens, and/or
optional a nucleic acid encoding said one or more antigens, and/or
optional the solid phase according to the invention.

In embodiments the one or more antigens can be the peptide disclosed herein.

In one embodiment the kit of the present invention optionally comprises:
one or more peptides selected from the group consisting of FIEDLLFNKVT (SEQ ID NO 1), IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) and FIEDLLFNKVTLADA (SEQ ID NO 10) or a sequence of at least 80%, preferably at least 90%, sequence identity thereto, and/or
optionally one or more antibodies, wherein said antibodies bind the amino acid sequence selected from the group consisting of FIEDLLFNKVT (SEQ ID NO 1), IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) and FIEDLLFNKVTLADA (SEQ ID NO 10) or a sequence of at least 80%, preferably at least 90%, sequence identity thereto.

In one embodiment the kit of the present invention optionally comprises a solid phase to which peptides with the amino acid sequence FIEDLLFNKVT (SEQ ID NO 1) or a sequence of at least 80%, preferably at least 90%, sequence identity thereto are immobilized.

In one embodiment the kit of the present invention optionally comprises a solid phase to which are immobilized:
one or more peptides with an amino acid sequence FIEDLLFNKVT (SEQ ID NO 1) or a sequence of at least 80%, preferably at least 90%, sequence identity thereto, preferably of 15 to 20 amino acids, and/or
one or more peptides selected from the group consisting of IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) and FIEDLLFNKVTLADA (SEQ ID NO 10) or a sequence of at least 80%, preferably at least 90%, sequence identity thereto, and/or
optionally one or more antibodies, wherein said antibodies bind the amino acid sequence of FIEDLLFNKVT (SEQ ID NO 1), IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) and FIEDLLFNKVTLADA (SEQ ID NO 10) or a sequence of at least 80%, preferably at least 90%, sequence identity thereto.

In one embodiment the kit of the present invention the kit comprises:
one or more antibodies, wherein said antibodies bind the amino acid sequence FIEDLLFNKVT (SEQ ID NO 1), IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) or FIEDLLFNKVTLADA (SEQ ID NO 10), or a sequence of at least 80%, preferably at least 90%, sequence identity thereto,
optionally one or more labels, such as fluorescent labels, either capable of binding said one or more antibodies, or linked to said one or more antibodies, and
optionally means for detecting said one or more labels.

In embodiments of the kit according to the invention the kit comprises one or more nucleic acid molecules encoding a peptide as described herein, wherein preferably said peptide has the amino acid sequence FIEDLLFNKVT (SEQ ID NO 1), IEDLLFNKV (SEQ ID NO 3), EDLLFNKVT (SEQ ID NO 4), FIEDLLFNKV (SEQ ID NO 6), IEDLLFNKVT (SEQ ID NO 7), SFIEDLLFNKVTLAD (SEQ ID NO 2), SKRSFIEDLLFNKVT (SEQ ID NO 9) or FIEDLLFNKVTLADA (SEQ ID NO 10), or a sequence of at least 80%, preferably at least 90%, sequence identity thereto.

The invention also relates to the use of a kit as described herein for the diagnosis of a SARS-CoV infection.

The invention also relates to the use of a kit as described herein for assessing the strength and/or quality of an immune response in a subject against a SARS-CoV infection.

The invention also relates to the use of a kit as described herein for assessing the risk of a subject in developing a severe acute respiratory syndrome (SARS) or other adverse event or severe medical condition associated with SARS-CoV infection (preferably with SARS-CoV-2).

The invention also relates to the use of a kit as described herein for assessing a level of T cell activation caused by one or more antigens, wherein the antigen preferably is, comprises and/or is derived from SARS-CoV, preferably SARS-CoV-2, more preferably SARS-CoV-2 spike glycoprotein, more preferably SARS-CoV-2 spike glycoprotein S-II, more preferably a peptide as described herein.

The invention also relates to the use of a kit as described herein for assessing functional avidity of T cells for one or more antigens, wherein the antigen preferably is, comprises and/or is derived from SARS-CoV, preferably SARS-CoV-2, more preferably SARS-CoV-2 spike glycoprotein, more preferably SARS-CoV-2 spike glycoprotein S-II, more preferably a peptide as described herein.

The embodiments described herein with reference to the kit of the present invention are intended to also relate to structural features of the components of the method as described herein.

The features of the kit as described herein may therefore also be used to characterize the method, and vice versa.

Additional embodiments relate to those presented in the claims below.

### DETAILED DESCRIPTION

In one aspect of the present invention there is provided methods for assessing a level of T cell activation by one or more antigens or for assessing functional avidity of T cells for one or more antigens, the methods comprising providing a cell population comprising T cells, contacting said cell population with one or more antigens in vitro, and determining a level of a T-cell receptor (TCR) complex and/or component thereof in a sub-set of T cells of said cell population.

The term “antigen” as used herein concurs with its ordinary meaning in the art. An antigen (Ag) is a molecule or molecular structure or any foreign particulate matter that can bind to an antibody or T-cell receptor. The presence of antigens in the body may trigger an immune response. Antigens can be, without limitation, proteins, peptides (amino acid chains), polysaccharides (chains of monosaccharides/simple sugars), lipids, or nucleic acids.

The term “T cell” as used herein concurs with its ordinary meaning in the art. A T cell is a type of lymphocyte and are one of the important white blood cells of the immune system and play a central role in the adaptive immune response. T cells can be distinguished from other lymphocytes by the presence of a T-cell receptor (TCR) on their cell surface. Groups of specific, differentiated T cell subtypes have a variety of important functions in controlling and shaping the immune response. One of these functions is immune-mediated cell death, and it is carried out by two major subtypes: CD8+ “killer” and CD4+ “helper” T cells. CD8+ T cells, also known as “killer T cells”, are cytotoxic, meaning that they are able to kill e.g. virus-infected cells, as well as cancer cells. CD8+ T cells are also able to use small signalling proteins, known as cytokines, to recruit other types of cells when mounting an immune response. A different population of T cells, the CD4+ T cells, function as “helper cells”. Unlike CD8+ killer T cells, the CD4+ helper T (TH) cells function by further activating memory B cells and cytotoxic T cells, which leads to a larger immune response. Regulatory T cells are yet another distinct population of T cells that provide the critical mechanism of tolerance, whereby immune cells are able to distinguish invading cells from “self”. This prevents immune cells from inappropriately reacting against one's own cells, known as an “autoimmune” response. For this reason, these regulatory T cells have also been called “suppressor” T cells. These same regulatory T cells can also be co-opted by cancer cells to prevent the recognition of, and an immune response against, tumor cells. The present invention may be applied on any given population of T cells, as described herein, and as known to a skilled person.

The T-cell receptor (TCR) is a protein complex found on the surface of T cells, or T lymphocytes, that is responsible for recognizing fragments of antigen as peptides bound to major histocompatibility complex (MHC) molecules. The binding between TCR and antigen peptides is typically of relatively low affinity and is degenerate: that is, many TCRs recognize the same antigen peptide and many antigen peptides are recognized by the same TCR.

Structurally, the TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells. Each chain is composed of two extracellular domains: Variable (V) region and a Constant (C) region, both of Immunoglobulin superfamily (IgSF) domain forming antiparallel s-sheets.

The Constant region is proximal to the cell membrane, followed by a transmembrane region and a short cytoplasmic tail, while the Variable region binds to the peptide/MHC complex. The variable domain of both the TCR α-chain and β-chain each have three hypervariable or complementarity-determining regions (CDRs). There is also an additional area of hypervariability on the β-chain (HV4) that does not normally contact antigen and, therefore, is not considered a CDR. The residues in these variable domains are located in two regions of the TCR, at the interface of the α- and β-chains and in the β-chain framework region that is thought to be in proximity to the CD3 signal-transduction complex.

CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition but has been shown to interact with superantigens.

Means for determining a level of a T-cell receptor (TCR) complex and/or component thereof are known to a skilled person.

In embodiments, the proportion of cells with reduced (low levels or downregulated) amounts of a TCR component, preferably CD3, indicate (or determine) a level of functional avidity.

As used herein, the term functional avidity preferably refers to a measure of a level of T cell activation caused by one or more antigens. The strength of T cell activation can therefore be assessed, preferably quantified, by assessing to what extent a T cell population, preferably a T helper cell population, can be activated by any given one or more antigens. As commonly used in the art, avidity typically refers to the accumulated strength of multiple affinities of individual non-covalent binding interactions, such as between a protein receptor and its ligand, and is commonly referred to as functional affinity. Avidity differs from affinity, which describes the strength of a single interaction.

The functional avidity relates in some embodiments to a measure of the strength of binding between the antigen and TCR. The functional avidity relates in some embodiments to a measure of effectiveness of a T-cell in inducing and/or supporting an immune response induced by the antigen. The functional avidity relates in some embodiments to a measure of immune response strength, said immune response mediated by CD4+ T helper cells. The functional avidity relates in some embodiments to a measure of immune response strength, capacity, speed and/or other quality of an immune response, said immune response mediated by the adaptive immune response. The functional avidity relates in some embodiments to a measure of immune response strength, in particular a measure of CD4+ T cell antigen-specific activation.

The stronger and longer the interaction of immune receptor and ligand (antigen), the stronger this downregulation of TCR component (eg CD3), and the greater the avidity of the TCR-antigen interaction, indicating a greater effectiveness in an immune response after binding between the TCR and antigen.

Flow cytometric analysis can be employed, by way of example, in order to determine a proportion of cells with reduced (low levels or downregulated) amounts of a TCR component, preferably CD3.

Using analysis of CD3 expression, functional avidities can be directly determined. Responses in the range up to about 5%, 10%, or 20% may be considered as only poorly functional, or as exhibiting low function. Here it can be assumed that recognition by memory T cells occurs with only low functional avidity, i.e. such T cells would for example recognize completely different antigenic peptides via their immune receptors, occurring for example also by chance. By way of further example, values of >50%, 60% or 70% or above indicate T cell reactivities with high functional avidity.

Flow cytometry is a widely used method for analyzing the expression of cell surface and intracellular molecules, characterizing and defining different cell types in a heterogeneous cell population, assessing the purity of isolated subpopulations, and analyzing cell size and volume. It allows simultaneous multi-parameter analysis of single cells. It is predominantly used to measure fluorescence intensity produced by fluorescent-labeled antibodies detecting proteins, or ligands that bind to specific cell-associated molecules such as propidium iodide binding to DNA. The staining procedure involves making a single-cell suspension from cell culture or tissue samples.

The cells are then incubated in tubes or microtiter plates with unlabeled or fluorochrome-labeled antibodies and analyzed on the flow cytometer Appropriate flow cytometric approaches can be elected by a skilled person based on common knowledge. For example, a flow cytometric setup may be employed as follows:

A sample source can include one or more containers, such as test tubes, that hold a sample to be analyzed. A fluid transfer system is provided in some embodiments, such as to aspirate the sample from the container and deliver the sample to the fluid nozzle. The sample is typically injected into a sheath fluid within the flow cytometer, which is provided by a sheath fluid source. In some embodiments a fluid nozzle is provided to generate the fluid stream and to inject the particles of the sample into the fluid stream. An example of a fluid nozzle is a flow cell. The fluid nozzle typically includes an aperture having a size selected to at least be larger than the sizes of particles of interest in the sample, but small enough to arrange the particles into a narrow stream. Ideally the particles are arranged in a single file or near single file arrangement so that a single particle, or a small number of particles (e.g., 1-3), can be passed through the light beam at a time. A light source (which can include one or more light sources) generates at least one light beam that is directed toward the fluid stream. Examples of light sources include a laser and an arc lamp. The light beam from the light source intersects the fluid stream. The particles contained in the light beam disturb the light beam and generate radiated light. The type and pattern of radiated light depends upon the type and size of the particles, but the radiated light can include forward scattered light, side scattered light, back scattered light, as well as fluorescent light (which occurs when light rays are absorbed and reemitted by the particle, which is detectable by the corresponding change in wavelength (i.e., colour) of the light rays). One or more detectors are provided to detect radiated light. For example, the detectors may include a detector arranged to detect forward scatter and florescence, a detector arranged to detect side scatter and florescence, and detector arranged to detect back scatter and florescence. One example of a detector is a photomultiplier.

Furthermore, cell sorting may also be applied to isolate T cells of interest, for example cell sorting allows to purify cell populations based on the presence or absence of specific physical characteristics. In flow cytometers with sorting capabilities, the instrument detects cells using parameters including cell size, morphology, protein expression, in particular cells labelled with particular markers, for example labeled (fluorescent) antibodies directed against specific targets, such as TCR receptor components, and then droplet technology is used to sort cells and recover the subsets for post-experimental use.

Flow cytometry cell sorters typically have a collection system unlike flow cytometry analyzers. The collection process starts when a sample is injected into a stream of sheath fluid that passes through the flow cell and laser intercepts. The stream then carries the cell through a vibrating nozzle which generates droplets with most containing either one cell or no cells. An electrical charging ring is placed just at the point where the stream breaks into droplets and a charge is placed on the ring based immediately prior to fluorescence intensity being measured; the opposite charge is trapped on the droplet as it breaks from the stream and the droplets are therefore charged. The charged droplets then fall through an electrostatic deflection system that diverts droplets into containers based on their charge. In some systems, the charge is applied directly to the stream, and the droplet breaking off retains charge of the same sign as the stream. The stream is then returned to neutral after the droplet breaks off. After collecting, these cells can be further cultured, manipulated, and studied.

Flow cytometry may also use the light properties scattered from cells or particles for identification or quantitative measurement of physical properties. Labels, dyes, and stains can be used for multi-parametric analysis (understand more properties about a cell). For example, immunophenotyping is the analysis of heterogeneous populations of cells using labeled antibodies and other fluorophore containing reagents such as dyes and stains. Alternatively, mass cytometry may be employed, which overcomes the fluorescent labeling limit by utilizing lanthanide isotopes attached to antibodies.

As used herein, the “contacting” of a cell population comprising T cells to an antigen can occur by any suitable means in the art. For example, a cell population obtained from a subject may be collected, optionally cultivated, and maintained in vitro or ex vivo, preferably in a liquid medium, into which an antigen can be applied. The antigen may also be immobilized to a solid phase, which is brought into contact with the relevant T cell containing population. Means for obtaining samples comprising T cells, and means for collecting and/or cultivating T cells, are known in the art.

The term “determining a level of a T-cell receptor (TCR) complex and/or component thereof” covers any suitable method to determine, preferably quantitatively or semi-quantitatively, an amount or level of a T-cell receptor (TCR) complex and/or component thereof. This determining preferably occurs in a sub-set of T cells of said cell population, such that a certain cell type, e.g. a subset of T cells is interrogated, and the proportion of T cells in said sub-set of cells in which the level of a T-cell receptor (TCR) complex and/or component thereof is below a first threshold value (TCR^{low} cells). By way of example, suitable flow cytometry approaches are disclosed herein, alternative means may be known to a skilled person. A threshold can therefore be established, using routine skills in the art, distinguishing between low or high levels of a T-cell receptor (TCR) complex and/or component thereof (preferably CD3). Within a cell population, the number of cells, or proportion of cells, that show levels above or below any given threshold, can be reliably determined. This output subsequently enables determination of functional T cell avidity to a specific antigen, and potentially selection of cells (e.g. using cell sorting techniques) with a desired functional T cell avidity, that can be selected and/or isolated for an appropriate downstream application, such as a therapeutic administration.

As used herein, the term “cross-reactivity” or “cross-immunoreactivity” shall mean that an antibody or receptor that binds an antigen is also effective against a region of another antigen.

For example, such a region can be an epitope which is specific area on the protein structure that is recognized by antibodies or T-cell receptors. Cross-immunoreactivity can occur between epitopes of different domains, or within the same domains, or between epitopes of different viruses or between epitopes of virus and other domains.

The innate immune system is the first line of defence against non-self pathogens, which is a non-specific immune response. The innate immune response consists of physical, chemical and cellular defences against pathogens. The main purpose of the innate immune response is to immediately prevent the spread and movement of foreign pathogens throughout the body.

The acquired immune system is the second line of defence against non-self pathogens. Adaptive immunity is also referred to as acquired immunity or specific immunity and is only found in vertebrates. The acquired immune response is specific to the pathogen presented. The term “adaptive immune response” and “acquired immune response” are interchangeable.

As used herein, the term “immune receptor” shall mean a receptor, usually on a cell membrane which binds to a substance and causes a response in the immune system, including pattern recognition receptors, Toll-like receptors, killer activated receptors, killer inhibitor receptors, complement receptors, Fc receptors, B cell receptors and T cell receptors.

The method according to the invention can be employed to identify high avidity T cells against tumor antigens ex vivo, for example for subsequent expansion in vitro, optionally followed by adoptive transfer into the patient. One of the main treatment modalities within cancer immunotherapy is adoptive T cell therapy (ACT). Using this approach, tumor-specific cytotoxic T cells are infused into cancer patients with the goal of recognizing, targeting, and destroying tumor cells. To mount an effective and targeted response, T cells must be able to recognize and target specific antigens presented in the context of major histocompatibility complex (MHC) proteins on the tumor that are not present or are poorly expressed on healthy tissue. The present invention therefore enable screening or assessment of T cells in order to select T cells with a high functional avidity to the cancer antigen.

Tumor-associated antigens (TAAs) were identified by seminal studies in the 1990s which conclusively demonstrated that immune cells could distinguish cancerous from healthy cells. Tumor-associated antigens can be classified into three major groups (Perica et al, Rambam Maimonides Med J. 2015 January; 6(1): e0004, Adoptive T Cell Immunotherapy for Cancer), including 1) Antigens over-expressed in tumors which are present on healthy tissue, but are over-expressed in cancer, often because they provide a growth advantage to the cell. These include the melanoma differentiation antigens, derived from differentiation proteins specific to the melanocyte lineage, are over-expressed in melanoma, and are recognized by TILs in many patients. 2) Neo-antigens arising from somatic mutations in cancer. 3) Cancer germline antigens, proteins that are normally expressed on germline cells, which reside in an immunoprivileged site and are thus less vulnerable to autoimmune T cell targeting. Any given TAA may for example be used in the method of the invention as the peptide of interest.

Through one of several techniques, T cells are harvested from a patient's blood or tumor, then stimulated to grow and expand in an in vitro culture system. After sufficient in vitro expansion, these cells are reinfused into the host, where they will hopefully mediate tumor destruction. The method of the invention therefore enables an additional step in existing techniques to identify effective T cells for adoptive therapy.

Protein modifications to the antigens or peptides of the present invention, which may occur through substitutions in amino acid sequence, and nucleic acid sequences encoding such molecules, are also included within the scope of the invention.

Substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the peptide. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, lie and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

As used herein, “nucleic acid” shall mean any nucleic acid molecule, including, without limitation, DNA, RNA and hybrids or modified variants thereof. An “exogenous nucleic acid” or “exogenous genetic element” relates to any nucleic acid introduced into the cell, which is not a component of the cells “original” or “natural” genome. Exogenous nucleic acids may be integrated or non-integrated, or relate to stably transfected nucleic acids.

As used herein, “peptide” may refer to peptides, polypeptides and proteins. In this invention, the peptides may be naturally occurring or recombinant (i.e., produced via recombinant DNA technology), and may contain mutations (e.g., point, insertion and deletion mutations) as well as other covalent modifications (e.g., glycosylation and labelling (via biotin, streptavidin, fluorescein, and radioisotopes)) or other molecular bonds to additional components.

In some embodiments of the invention the peptide, preferably according to sequences disclosed herein, may comprise a 0 to 10 amino acid addition or deletion at the N and/or C terminus of a sequence. As used herein the terms “1-10 additional amino acids at the N and/or C-termini” or “deletion at the N and/or C terminus of a sequence” means that the polypeptide may have:
a) 1, 2, 3, 4, 5, 6, 7, 8, 9 or 10 additional amino acids at its N terminus and 1, 2, 3, 4, 5, 6, 7, 8, 9 or 10 amino acids deleted at its C terminus, or
b) 1, 2, 3, 4, 5, 6, 7, 8, 9 or 10 additional amino acids at its C terminus and 1, 2, 3, 4, 5, 6, 7, 8, 9 or 10 nucleotides deleted at its N terminus, or
c) 1, 2, 3, 4, 5, 6, 7, 8, 9 or 10 additional amino acids at its N terminus and 1, 2, 3, 4, 5, 6, 7, 8, 9 or 10 additional amino acids at its N terminus, or
d) 1, 2, 3, 4, 5, 6, 7, 8, 9 or 10 amino acids deleted at its N terminus and 1, 2, 3, 4, 5, 6, 7, 8, 9 or 10 amino acids deleted at its C terminus.

The same principle applies for “1-20 additional amino acids at the N and/or C-termini” and “1-30 additional amino acids at the N and/or C-termini”. As used herein, the “patient” or “subject” may be a vertebrate. In the context of the present invention, the term “subject” includes both humans and animals, particularly mammals, and other organisms.

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

The prophylactic therapy as described herein is intended to encompass prevention or reduction of risk of Coronavirus infection, due to a reduced likelihood of Coronavirus infection of cells via interaction with the ACE2 protein after treatment with the agents described herein.

In one embodiment, a patient with symptoms of an infectious disease may be assessed using the method of the invention. A “patient with symptoms of an infectious disease” is a subject who presents with one or more of, without limitation, fever, diarrhea, fatigue, muscle aches, coughing, if have been bitten by an animal, having trouble breathing, severe headache with fever, rash or swelling, unexplained or prolonged fever or vision problems. Other symptoms may be fever and chills, very low body temperature, decreased output of urine (oliguria), rapid pulse, rapid breathing, nausea and vomiting. In preferred embodiments the symptoms of an infectious disease are fever, diarrhea, fatigue, muscle aches, rapid pulse, rapid breathing, nausea and vomiting and/or coughing.

In one embodiment, a patient with symptoms of a viral infection of the respiratory tract may be assessed using the method of the invention. As used herein, a patient with “symptoms of a viral infection of the respiratory tract” is a subject who presents with one or more of, without limitation, cold-like symptoms or flu-like illnesses, such as fever, cough, runny nose, sneezing, sore throat, having trouble breathing, headache, muscle aches, fatigue, rapid pulse, rapid breathing, nausea and vomiting, lack of taste and/or smell and/or malaise (feeling unwell).

In some embodiments, symptoms of infection with a SARS-virus are fever, sore throat, cough, myalgia or fatigue, and in some embodiments, additionally, sputum production, headache, hemoptysis and/or diarrhea. In some embodiments, symptoms of an infection with a SARS-coronavirus, for example SARS-CoV-2, are fever, sore throat, cough, lack of taste and/or smell, shortness of breath and/or fatigue.

In one embodiment, a patient that is at risk of developing a severe acute respiratory syndrome (SARS) may be assessed using the method of the invention. As used herein, the term “a patient that is at risk of developing a severe acute respiratory syndrome (SARS)” relates to a subject, preferably distinct from any given person in the general population, who has an increased (e.g. above-average) risk of developing SARS. In some embodiments, the patient has symptoms of SARS or symptoms of a SARS Coronavirus infection. In some embodiments, the patient has no symptoms of SARS or symptoms of a SARS Coronavirus infection. In some embodiments, the subject has been in contact with people with SARS Coronavirus infections or symptoms. In some embodiments, the person at risk of developing SARS has been tested for the presence of a SARS Coronavirus infection. In some embodiments, the person at risk of developing SARS has tested positive for the presence of a SARS Coronavirus infection, preferably a coronavirus infection.

In some embodiments, the patient that has or is at risk of developing a severe acute respiratory syndrome (SARS) has a coronavirus infection. Coronaviruses are a group of related viruses that cause diseases in mammals and birds. The scientific name for coronavirus is Orthocoronavirinae or Coronavirinae. Coronavirus belongs to the family of Coronaviridae. The family is divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts.

In humans, coronaviruses cause respiratory tract infections that can be mild, such as some cases of the common cold, and others that can be lethal, such as SARS, MERS, and COVID-19. Coronaviruses are enveloped viruses with a positive-sense single-stranded RNA genome and a nucleocapsid of helical symmetry. The genome size of coronaviruses ranges from approximately 27 to 34 kilobases, the largest among known RNA viruses.

Various species of human coronaviruses are known, such as, without limitation, Human coronavirus OC43 (HCoV-OC43), of the genus β-CoV, Human coronavirus HKU1 (HCoV-HKU1), of the genus β-CoV, Human coronavirus 229E (HCoV-229E), α-CoV, Human coronavirus NL63 (HCoV-NL63), α-CoV, Middle East respiratory syndrome-related coronavirus (MERS-CoV), Severe acute respiratory syndrome coronavirus (SARS-CoV), Severe acute respiratory syndrome coronavirus 2 (SARS-CoV-2).

Coronaviruses vary significantly in risk factor. Some can kill more than 30% of those infected (such as MERS-CoV), and some are relatively harmless, such as the common cold. Coronaviruses cause colds with major symptoms, such as fever, and a sore throat, e.g. from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). Coronaviruses can also cause SARS.

As used herein, “diagnosis” in the context of the present invention relates to the recognition and (early) detection of a clinical condition of a subject associated with a SARS-CoV infection. Also the assessment of the possibilities of developing adverse symptoms may be encompassed by the term “diagnosis”.

“Prognosis” relates to the prediction of an outcome or a specific risk for a subject based on an a clinical condition of a subject linked to a SARS-CoV infection. This may also include an estimation of the chance of recovery or the chance of an adverse outcome for said subject.

In the present invention, the term “risk assessment” relates to the grouping of subjects into one or more risk groups according to their further prognosis. Risk assessment also relates to stratification for applying preventive and/or therapeutic measures. Risk assessment may also apply to “therapy stratification”, in particular relates to grouping or classifying patients into different groups, such as risk groups or therapy groups that receive certain differential therapeutic measures depending on their classification. The term “therapy stratification” also relates to grouping or classifying patients with infections or having symptoms of an infectious disease into a group that are not in need to receive certain therapeutic measures.

As used herein, viral spike protein refers to a transmembrane protein ranging from 1 to 160, or 200, or 250 or 300 or 350 or 400 or 450 or 500 or 600 or 700 or 800 or 900 or 1000 amino acids. Viral spike protein can be highly glycosylated. SARS-CoV diversity is reflected in the variable spike protein, which have evolved into forms differing in their receptor interactions and their response to various environmental triggers of virus-cell membrane fusion. SARS-CoV-2 infects human epithelial cells through interaction with the human ACE2 receptor. A notable distinction between the spike proteins of different coronaviruses is whether it is cleaved or not during assembly and exocytosis of virons. With some exceptions, in most alphacoronaviruses and the betacoronavirus SARS-CoV, the virions harbor a spike protein that is uncleaved, whereas in some beta- and all gamma-coronaviruses the protein is found cleaved between the S1 and S2 domains, typically by furin, a Golgi-resident host protease.

A mutation of SARS-CoV-2 can be caused by mutation of the spike protein of SARS-CoV-2. For example, the spike protein of SARS-CoV-2 acquired a deletion 69-70, deletion 144, N501Y, A570D, D614G, P681H, T716I, S982A or D1118H. Among these, the D614G mutation confers greater infectivity.

As used herein, the “immunogenic composition” refers to a foreign substance, such as an antigen, to provoke an immune response in the body of a human or other animal. In other words, immunogenicity is the ability to induce a humoral and/or cell-mediated immune responses.

As used herein, the “vaccine” refers to a biological preparation that provides active acquired immunity to a particular infectious disease. A vaccine typically contains an agent that resembles a disease-causing microorganism and is often made from weakened or killed forms of the microbe, its toxins, or one of its surface proteins, or with nucleic acid molecules encoding a relevant antigen. The agent stimulates the body's immune system to recognize the agent as a threat, destroy it, and to further recognize and destroy any of the microorganisms associated with that agent that it may encounter in the future.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”. Thus, the terms “comprising”/“including”/“having” mean that any further component (or likewise features, integers, steps and the like) can/may be present. The term “consisting of” means that no further component (or likewise features, integers, steps and the like) is present.

### Sequences of the Invention:

| SEQ ID NO | Sequence | Description |
|---|---|---|
| 1 | FIEDLLFNKVT | Exemplary iCOPE |
| | | sequence 1 |
| 2 | SFIEDLLFNKVTLAD | Exemplary iCOPE |
| | | sequence 2 |
| 3 | IEDLLFNKV | Exemplary iCOPE |
| | | sequence 3 |
| 4 | EDLLFNKVT | Exemplary iCOPE |
| | | sequence 4 |
| 5 | LADA | Flanking |
| | | sequence 1 |
| 6 | FIEDLLFNKV | Exemplary iCOPE |
| | | sequence 5 |
| 7 | IEDLLFNKVT | Exemplary iCOPE |
| | | sequence 6 |
| 8 | SKRS | Flanking |
| | | sequence 2 |
| 9 | SKRSFIEDLLFNKVT | Exemplary iCOPE |
| | | sequence 7 |
| 10 | FIEDLLFNKVTLADA | Exemplary iCOPE |
| | | sequence 8 |
| 11 | MFVFLVLLPLVSSQCVNLTTRTQLPPAYTNSFTRGVYYPDKV | WUHAN |
| | FRSSVLHSTQDLFLPFFSNVTWFHAIHVSGTNGTKRFDNPVL | Sequence |
| | PFNDGVYFASTEKSNIIRGWIFGTTLDSKTQSLLIVNNATNVVI | >YP_009724390. |
| | KVCEFQFCNDPFLGVYYHKNNKSWMESEFRVYSSANNCTF | 1 surface |
| | EYVSQPFLMDLEGKQGNFKNLREFVFKNIDGYFKIYSKHTPI | glycoprotein |
| | NLVRDLPQGFSALEPLVDLPIGINITRFQTLLALHRSYLTPGDS | [Severe acute |
| | SSGWTAGAAAYYVGYLQPRTFLLKYNENGTITDAVDCALDP | respiratory |
| | LSETKCTLKSFTVEKGIYQTSNFRVQPTESIVRFPNITNLCPF | syndrome |
| | GEVFNATRFASVYAWNRKRISNCVADYSVLYNSASFSTFKC | coronavirus 2] |
| | YGVSPTKLNDLCFTNVYADSFVIRGDEVRQIAPGQTGKIADY | |
| | NYKLPDDFTGCVIAWNSNNLDSKVGGNYNYLYRLFRKSNLK | |
| | PFERDISTEIYQAGSTPCNGVEGFNCYFPLQSYGFQPTNGV | |
| | GYQPYRVVVLSFELLHAPATVCGPKKSTNLVKNKCVNFNFN | |
| | GLTGTGVLTESNKKFLPFQQFGRDIADTTDAVRDPQTLEILDI | |
| | TPCSFGGVSVITPGTNTSNQVAVLYQDVNCTEVPVAIHADQL | |
| | TPTWRVYSTGSNVFQTRAGCLIGAEHVNNSYECDIPIGAGIC | |
| | ASYQTQTNSPRRARSVASQSIIAYTMSLGAENSVAYSNNSIAI | |
| | PTNFTISVTTEILPVSMTKTSVDCTMYICGDSTECSNLLLQYG | |
| | SFCTQLNRALTGIAVEQDKNTQEVFAQVKQIYKTPPIKDFGG | |
| | FNFSQILPDPSKPSKRSFIEDLLFNKVTLADAGFIKQYGDCLG | |
| | DIAARDLICAQKFNGLTVLPPLLTDEMIAQYTSALLAGTITSG | |
| | WTFGAGAALQIPFAMQMAYRFNGIGVTQNVLYENQKLIANQ | |
| | FNSAIGKIQDSLSSTASALGKLQDVVNQNAQALNTLVKQLSS | |
| | NFGAISSVLNDILSRLDKVEAEVQIDRLITGRLQSLQTYVTQQ | |
| | LIRAAEIRASANLAATKMSECVLGQSKRVDFCGKGYHLMSFP | |
| | QSAPHGVVFLHVTYVPAQEKNFTTAPAICHDGKAHFPREGV | |
| | FVSNGTHWFVTQRNFYEPQIITTDNTFVSGNCDVVIGIVNNT | |
| | VYDPLQPELDSFKEELDKYFKNHTSPDVDLGDISGINASVVNI | |
| | QKEIDRLNEVAKNLNESLIDLQELGKYEQYIKWPWYIWLGFIA | |
| | GLIAIVMVTIMLCCMTSCCSCLKGCCSCGSCCKFDEDDSEPV | |
| | LKGVKLHYT | |
| 12 | MFVFLVLLPLVSSQCVNLTTRTQLPPAYTNSFTRGVYYPDKV | B.1.1.7 (Alpha) |
| | FRSSVLHSTQDLFLPFFSNVTWFHAISGTNGTKRFDNPVLPF | >QQQ47833.1 |
| | NDGVYFASTEKSNIIRGWIFGTTLDSKTQSLLIVNNATNVVIKV | surface |
| | CEFQFCNDPFLGVYHKNNKSWMESEFRVYSSANNCTFEYV | glycoprotein |
| | SQPFLMDLEGKQGNFKNLREFVFKNIDGYFKIYSKHTPINLVR | [Severe acute |
| | DLPQGFSALEPLVDLPIGINITRFQTLLALHRSYLTPGDSSSG | respiratory |
| | WTAGAAAYYVGYLQPRTFLLKYNENGTITDAVDCALDPLSET | syndrome |
| | KCTLKSFTVEKGIYQTSNFRVQPTESIVRFPNITNLCPFGEVF | coronavirus 2] |
| | NATRFASVYAWNRKRISNCVADYSVLYNSASFSTFKCYGVS | |
| | PTKLNDLCFTNVYADSFVIRGDEVRQIAPGQTGKIADYNYKL | |
| | PDDFTGCVIAWNSNNLDSKVGGNYNYLYRLFRKSNLKPFER | |
| | DISTEIYQAGSTPCNGVEGFNCYFPLQSYGFQPTYGVGYQP | |
| | YRVVVLSFELLHAPATVCGPKKSTNLVKNKCVNFNFNGLTGT | |
| | GVLTESNKKFLPFQQFGRDIDDTTDAVRDPQTLEILDITPCSF | |
| | GGVSVITPGTNTSNQVAVLYQGVNCTEVPVAIHADQLTPTW | |
| | RVYSTGSNVFQTRAGCLIGAEHVNNSYECDIPIGAGICASYQ | |
| | TQTNSHRRARSVASQSIIAYTMSLGAENSVAYSNNSIAIPINF | |
| | TISVTTEILPVSMTKTSVDCTMYICGDSTECSNLLLQYGSFCT | |
| | QLNRALTGIAVEQDKNTQEVFAQVKQIYKTPPIKDFGGFNFS | |
| | QILPDPSKPSKRSFIEDLLFNKVTLADAGFIKQYGDCLGDIAA | |
| | RDLICAQKFNGLTVLPPLLTDEMIAQYTSALLAGTITSGWTFG | |
| | AGAALQIPFAMQMAYRFNGIGVTQNVLYENQKLIANQFNSAI | |
| | GKIQDSLSSTASALGKLQDVVNQNAQALNTLVKQLSSNFGAI | |
| | SSVLNDILARLDKVEAEVQIDRLITGRLQSLQTYVTQQLIRAAE | |
| | IRASANLAATKMSECVLGQSKRVDFCGKGYHLMSFPQSAPH | |
| | GVVFLHVTYVPAQEKNFTTAPAICHDGKAHFPREGVFVSNG | |
| | THWFVTQRNFYEPQIITTHNTFVSGNCDVVIGIVNNTVYDPLQ | |
| | PELDSFKEELDKYFKNHTSPDVDLGDISGINASVVNIQKEIDR | |
| | LNEVAKNLNESLIDLQELGKYEQYIKWPWYIWLGFIAGLIAIVM | |
| | VTIMLCCMTSCCSCLKGCCSCGSCCKFDEDDSEPVLKGVKL | |
| | HYT | |
| 13 | MFVFLVLLPLVSSQCVNFTTRTQLPPAYTNSFTRGVYYPDKV | B.1.351 (Beta) |
| | FRSSVLHSTQDLFLPFFSNVTWFHAIHVSGTNGTKRFANPVL | >QWW93436.1 |
| | PFNDGVYFASTEKSNIIRGWIFGTTLDSKTQSLLIVNNATNVVI | surface |
| | KVCEFQFCNDPFLGVYYHKNNKSWMESEFRVYSSANNCTF | glycoprotein |
| | EYVSQPFLMDLEGKQGNFKNLREFVFKNIDGYFKIYSKHTPI | [Severe acute |
| | NLVRGLPQGFSALEPLVDLPIGINITRFQTLHRSYLTPGDSSS | respiratory |
| | GWTAGAAAYYVGYLQPRTFLLKYNENGTITDAVDCALDPLS | syndrome |
| | ETKCTLKSFTVEKGIYQTSNFRVQPTESIVRFPNITNLCPFGE | coronavirus 2] |
| | VFNATRFASVYAWNRKRISNCVADYSVLYNSASFSTFKCYG | |
| | VSPTKLNDLCFTNVYADSFVIRGDEVRQIAPGQTGNIADYNY | |
| | KLPDDFTGCVIAWNSNNLDSKVGGNYNYLYRLFRKSNLKPF | |
| | ERDISTEIYQAGSTPCNGVKGFNCYFPLQSYGFQPTYGVGY | |
| | QPYRVVVLSFELLHAPATVCGPKKSTNLVKNKCVNFNFNGLT | |
| | GTGVLTESNKKFLPFQQFGRDIADTTDAVRDPQTLEILDITPC | |
| | SFGGVSVITPGTNTSNQVAVLYQGVNCTEVPVAIHADQLTPT | |
| | WRVYSTGSNVFQTRAGCLIGAEHVNNSYECDIPIGAGICASY | |
| | QTQTNSPRRARSVASQSIIAYTMSLGVENSVAYSNNSIAIPTN | |
| | FTISVTTEILPVSMTKTSVDCTMYICGDSTECSNLLLQYGSFC | |
| | TQLNRALTGIAVEQDKNTQEVFAQVKQIYKTPPIKDFGGFNF | |
| | SQILPDPSKPSKRSFIEDLLFNKVTLADAGFIKQYGDCLGDIA | |
| | ARDLICAQKFNGLTVLPPLLTDEMIAQYTSALLAGTITSGWTF | |
| | GAGAALQIPFAMQMAYRFNGIGVTQNVLYENQKLIANQFNSA | |
| | IGKIQDSLSSTASALGKLQDVVNQNAQALNTLVKQLSSNFGAI | |
| | SSVLNDILSRLDKVEAEVQIDRLITGRLQSLQTYVTQQLIRAAE | |
| | IRASANLAATKMSECVLGQSKRVDFCGKGYHLMSFPQSAPH | |
| | GVVFLHVTYVPAQEKNFTTAPAICHDGKAHFPREGVFVSNG | |
| | THWFVTQRNFYEPQIITTDNTFVSGNCDVVIGIVNNTVYDPLQ | |
| | PELDSFKEELDKYFKNHTSPDVDLGDISGINASVVNIQKEIDR | |
| | LNEVAKNLNESLIDLQELGKYEQYIKWPWYIWLGFIAGLIAIVM | |
| | VTIMLCCMTSCCSCLKGCCSCGSCCKFDEDDSEPVLKGVKL | |
| | HYT | |
| 14 | MFVFLVLLPLVSSQCVNLRTRTQLPPAYTNSFTRGVYYPDKV | B.1.617.2 (Delta) |
| | FRSSVLHSTQDLFLPFFSNVTWFHAIHVSGTNGTKRFDNPVL | |
| | PFNDGVYFASTEKSNIIRGWIFGTTLDSKTQSLLIVNNATNVVI | |
| | KVCEFQFCNDPFLGVYYHKNNKSWMESEVYSSANNCTFEY | |
| | VSQPFLMDLEGKQGNFKNLREFVFKNIDGYFKIYSKHTPINLV | |
| | RDLPQGFSALEPLVDLPIGINITRFQTLLALHRSYLTPGDSSS | |
| | GWTAGAAAYYVGYLQPRTFLLKYNENGTITDAVDCALDPLS | |
| | ETKCTLKSFTVEKGIYQTSNFRVQPTESIVRFPNITNLCPFGE | |
| | VFNATRFASVYAWNRKRISNCVADYSVLYNSASFSTFKCYG | |
| | VSPTKLNDLCFTNVYADSFVIRGDEVRQIAPGQTGKIADYNY | |
| | KLPDDFTGCVIAWNSNNLDSKVGGNYNYRYRLFRKSNLKPF | |
| | ERDISTEIYQAGSKPCNGVEGFNCYFPLQSYGFQPTNGVGY | |
| | QPYRVVVLSFELLHAPATVCGPKKSTNLVKNKCVNFNFNGLT | |
| | GTGVLTESNKKFLPFQQFGRDIADTTDAVRDPQTLEILDITPC | |
| | SFGGVSVITPGTNTSNQVAVLYQGVNCTEVPVAIHADQLTPT | |
| | WRVYSTGSNVFQTRAGCLIGAEHVNNSYECDIPIGAGICASY | |
| | QTQTNSRRRARSVASQSIIAYTMSLGAENSVAYSNNSIAIPTN | |
| | FTISVTTEILPVSMTKTSVDCTMYICGDSTECSNLLLQYGSFC | |
| | TQLNRALTGIAVEQDKNTQEVFAQVKQIYKTPPIKDFGGFNF | |
| | SQILPDPSKPSKRSFIEDLLFNKVTLADAGFIKQYGDCLGDIA | |
| | ARDLICAQKFNGLTVLPPLLTDEMIAQYTSALLAGTITSGWTF | |
| | GAGAALQIPFAMQMAYRFNGIGVTQNVLYENQKLIANQFNSA | |
| | IGKIQDSLSSTASALGKLQNVVNQNAQALNTLVKQLSSNFGAI | |
| | SSVLNDILSRLDKVEAEVQIDRLITGRLQSLQTYVTQQLIRAAE | |
| | IRASANLAATKMSECVLGQSKRVDFCGKGYHLMSFPQSAPH | |
| | GVVFLHVTYVPAQEKNFTTAPAICHDGKAHFPREGVFVSNG | |
| | THWFVTQRNFYEPQIITTDNTFVSGNCDVVIGIVNNTVYDPLQ | |
| | PELDSFKEELDKYFKNHTSPDVDLGDISGINASVVNIQKEIDR | |
| | LNEVAKNLNESLIDLQELGKYEQYIKWPWYIWLGFIAGLIAIVM | |
| | VTIMLCCMTSCCSCLKGCCSCGSCCKFDEDDSEPVLKGVKL | |
| | HYT | |
| 15 | MFVFLVLLPLVSSQCVNLTTRTQLPPAYTNSFTRGVYYPDKV | B.1.1.529 |
| | FRSSVLHSTQDLFLPFFSNVTWFHVISGTNGTKRFDNPVLPF | (Omicron) |
| | NDGVYFASIEKSNIIRGWIFGTTLDSKTQSLLIVNNATNVVIKV | PDB: 7Q09_B |
| | CEFQFCNDPFLDHKNNKSWMESEFRVYSSANNCTFEYVSQ | |
| | PFLMDLEGKQGNFKNLREFVFKNIDGYFKIYSKHTPIIVREPE | |
| | DLPQGFSALEPLVDLPIGINITRFQTLLALHRSYLTPGDSSSG | |
| | WTAGAAAYYVGYLQPRTFLLKYNENGTITDAVDCALDPLSET | |
| | KCTLKSFTVEKGIYQTSNFRVQPTESIVRFPNITNLCPFDEVF | |
| | NATRFASVYAWNRKRISNCVADYSVLYNLAPFFTFKCYGVS | |
| | PTKLNDLCFTNVYADSFVIRGDEVRQIAPGQTGNIADYNYKL | |
| | PDDFTGCVIAWNSNKLDSKVSGNYNYLYRLFRKSNLKPFER | |
| | DISTEIYQAGNKPCNGVAGFNCYFPLRSYSFRPTYGVGHQP | |
| | YRVVVLSFELLHAPATVCGPKKSTNLVKNKCVNFNFNGLKGT | |
| | GVLTESNKKFLPFQQFGRDIADTTDAVRDPQTLEILDITPCSF | |
| | GGVSVITPGTNTSNQVAVLYQGVNCTEVPVAIHADQLTPTW | |
| | RVYSTGSNVFQTRAGCLIGAEYVNNSYECDIPIGAGICASYQ | |
| | TQTKSHGSASSVASQSIIAYTMSLGAENSVAYSNNSIAIPTNF | |
| | TISVTTEILPVSMTKTSVDCTMYICGDSTECSNLLLQYGSFCT | |
| | QLKRALTGIAVEQDKNTQEVFAQVKQIYKTPPIKYFGGFNFS | |
| | QILPDPSKPSKRSFIEDLLFNKVTLADAGFIKQYGDCLGDIAA | |
| | RDLICAQKFKGLTVLPPLLTDEMIAQYTSALLAGTITSGWTFG | |
| | AGAALQIPFAMQMAYRFNGIGVTQNVLYENQKLIANQFNSAI | |
| | GKIQDSLSSTASALGKLQDVVNHNAQALNTLVKQLSSKFGAI | |
| | SSVLNDIFSRLDPPEAEVQIDRLITGRLQSLQTYVTQQLIRAA | |
| | EIRASANLAATKMSECVLGQSKRVDFCGKGYHLMSFPQSAP | |
| | HGVVFLHVTYVPAQEKNFTTAPAICHDGKAHFPREGVFVSN | |
| | GTHWFVTQRNFYEPQIITTDNTFVSGNCDVVIGIVNNTVYDPL | |
| | QPELDSFKEELDKYFKNHTSPDVDLGDISGINASVVNIQKEID | |
| | RLNEVAKNLNESLIDLQELGKYEQGSGYIPEAPRDGQAYVRK | |
| | DGEWVLLSTFLGRSLEVLFQGPGHHHHHHHHSAWSHPQFE | |
| | KGGGSGGGGSGGSAWSHPQFEK | |
| 16 | SKPSKRSFIEDLLFNKVTLADAGFI | Exemplary iCOPE |
| | | sequence 9 |
| 17 | EDLLFNKV | Exemplary iCOPE |
| | | sequence 10 |
| 18 | SFIEDLLF | Exemplary iCOPE |
| | | sequence 11 |
| 19 | PSKPSKRSFIEDLLFNKV | biotinylated |
| | | peptide |
| 20 | KRSFIEDLLFNKVTL | Exemplary iCOPE |
| | | sequence 12 |
| 21 | RSFIEDLLFNKVTLA | Exemplary iCOPE |
| | | sequence 13 |
| 22 | VNNTVYDPLQPELDS | Control peptide |
| 23 | KQYTSACKTIEDALRLSAHLETNDVSSMLTFDSNAFSLANVT | NL63 |
| | SFGDYNLSSVLPQRNIRSSRIAGRSALEDLLFSKVVTSGLGT | |
| 24 | KQYTSACKTIEDALRNSAMLESADVSEMLTFDKKAFTLANVS | 229E |
| | SFGDYNLSSVIPSLPRSGSRVAGRSAIEDILFSKLVTSGLGT | |
| 25 | SEYGTFCDNINSILNEVNDLLDITQLQVANALMQGVTLSSNLN | HKU1 |
| | TNLHSDVDNIDFKSLLGCLGSQCGSSSRSLLEDLLFNKVKLS | |
| | DVGF | |
| 26 | VEYGSFCDNINAILTEVNELLDTTQLQVANSLMNGVTLSTKLK | OC43 |
| | DGVNFNVDDINFAPVLGCLGSECSKASSRSAIEDLLFDKVKL | |
| | SDVGF | |
| 27 | REYGQFCSKINQALHGANLRQDDSVRNLFASVKSSQSSPIIP | MERS |
| | GFGGDFNLTLLEPVSISTGSRSARSAIEDLLFDKVTIADPGY | |
| 28 | LQYGSFCTQLNRALSGIAAEQDRNTREVFAQVKQMYKTPTL | SARS |
| | KYFGGFNFSQILPDPLKPTKRSFIEDLLFNKVTLADAGF | |
| 29 | LQYGSFCTQLNRALTGIAVEQDKNTQEVFAQVKQIYKTPPIK | SARS-COV-2 |
| | DFGGFNFSQILPDPSKPSKRSFIEDLLFNKVTLADAGF | |

The following figures and examples are presented to describe particular embodiments of the invention, without being limiting in scope.

### FIGURES

### Brief Description of the Figures

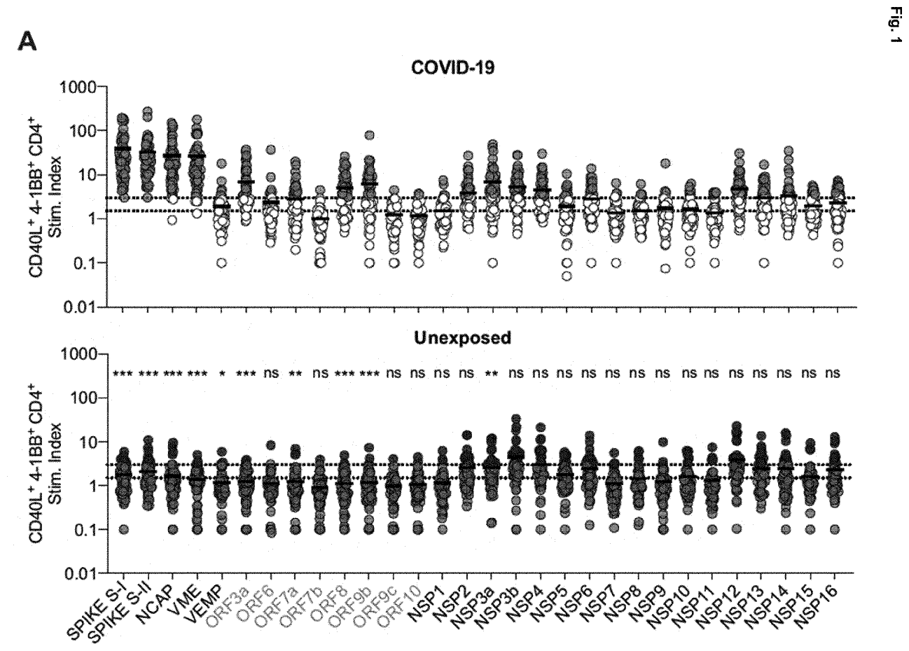

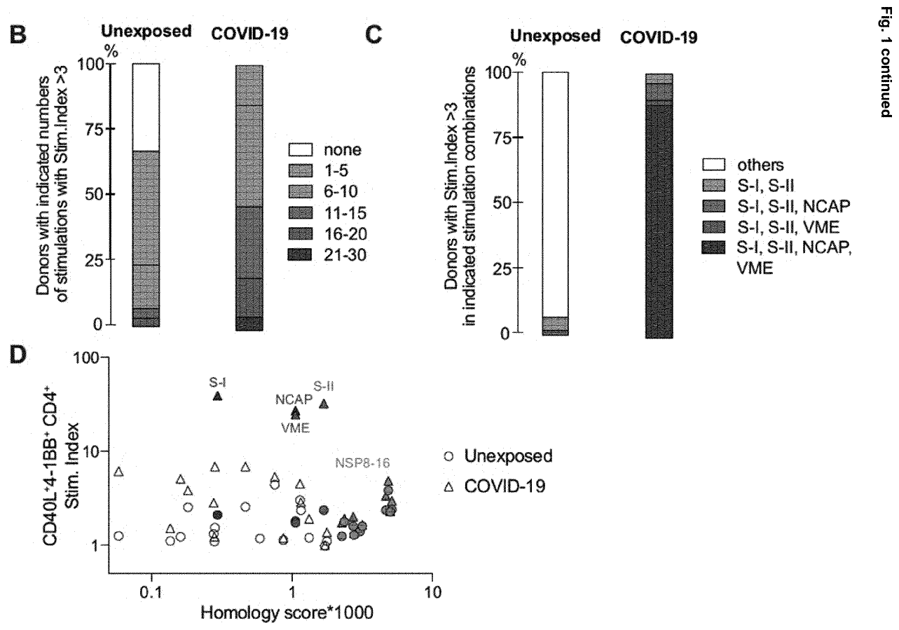

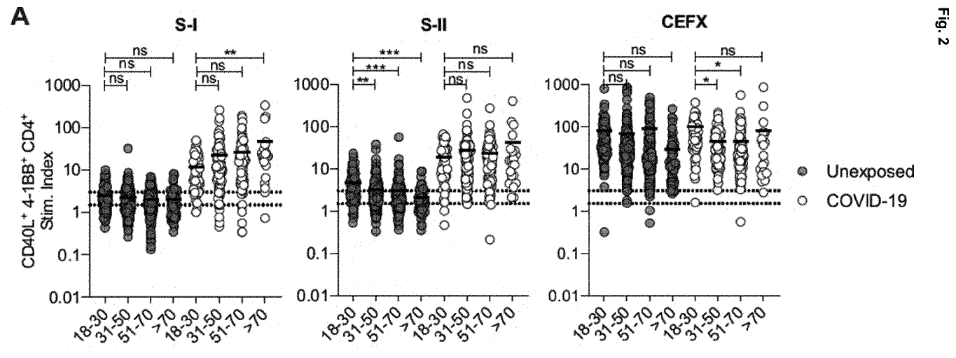

FIG. 1: CD4+ T cell cross-reactivity against the SARS-CoV-2 orfeome

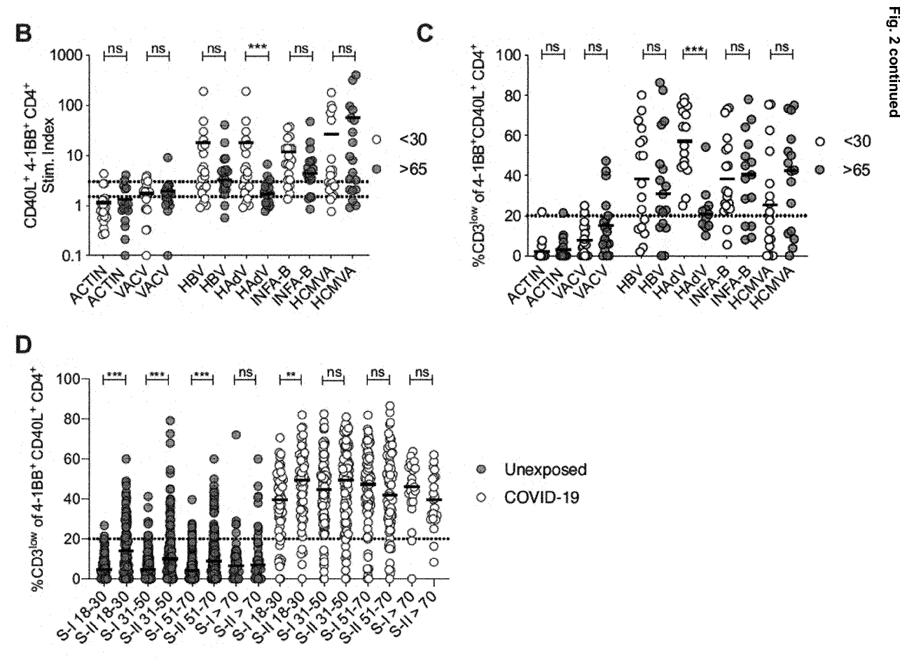

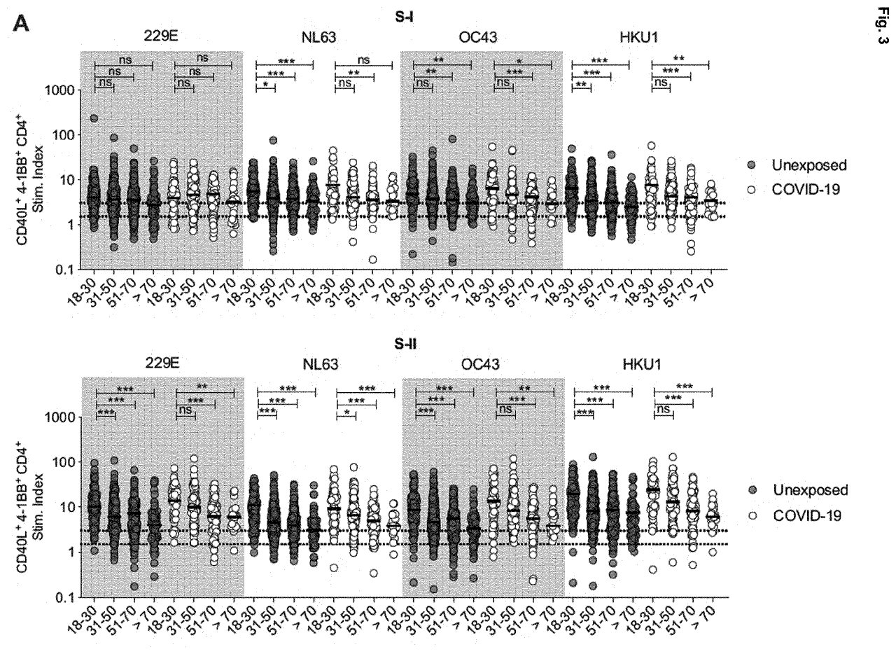

FIG. 2: Magnitude of SARS-CoV-2-cross-reactivity decreases with age

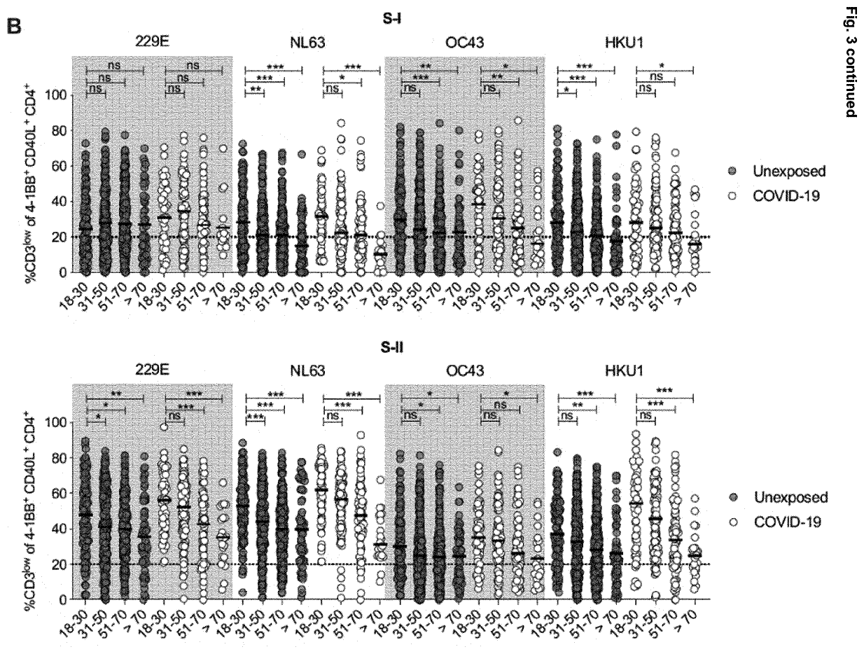

FIG. 3: High functional avidity T cells to spike S-II from HCoVs decrease with age

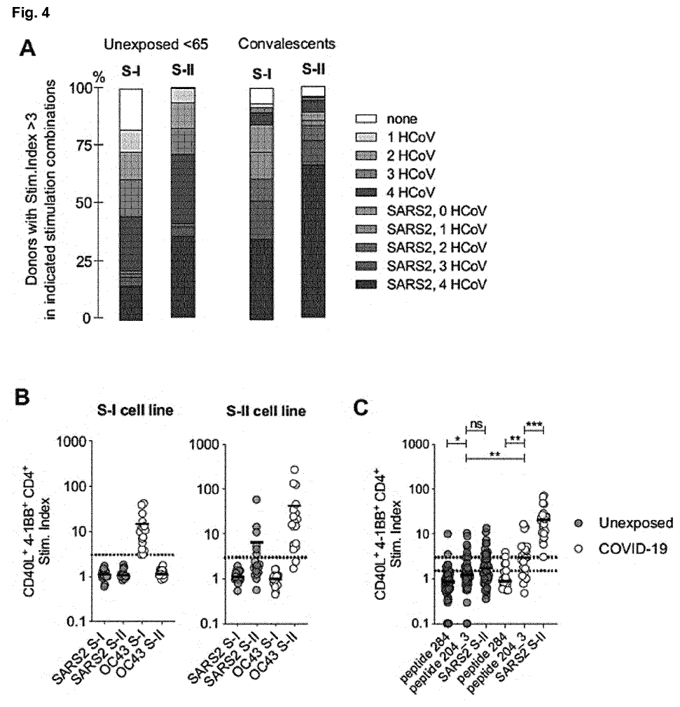

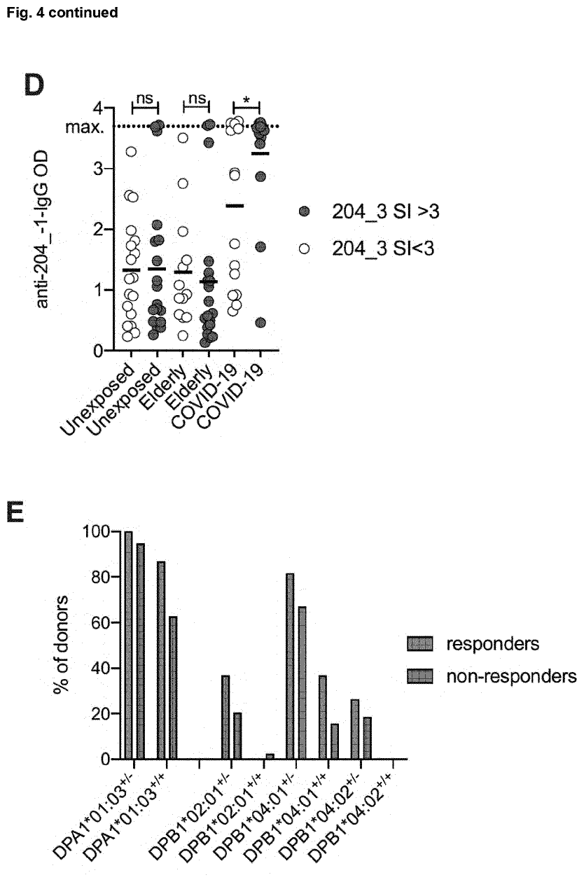

FIG. 4: Peptide 204_3 (iCope) constitutes an immunodominant epitope of SARS-CoV-2 T cell (cross)-reactivity

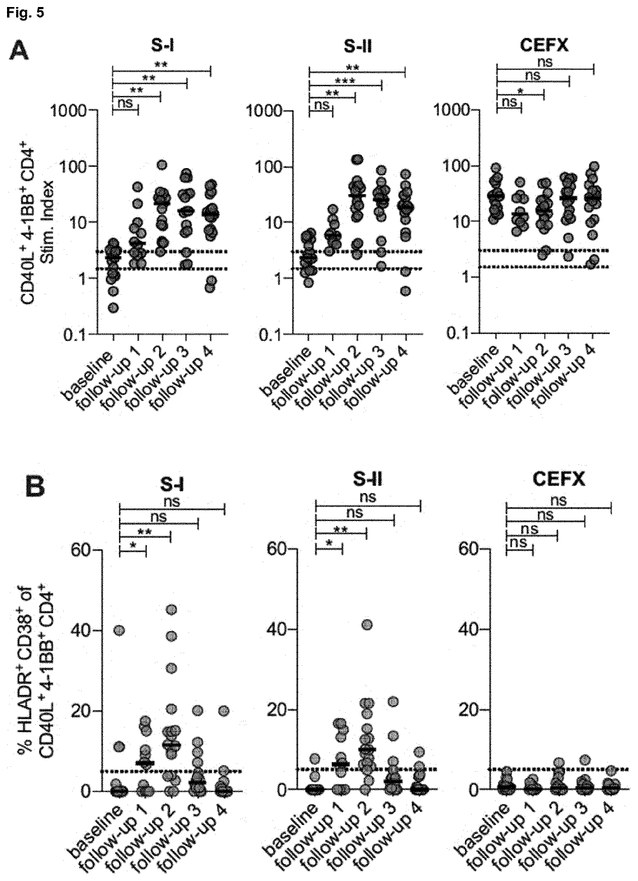

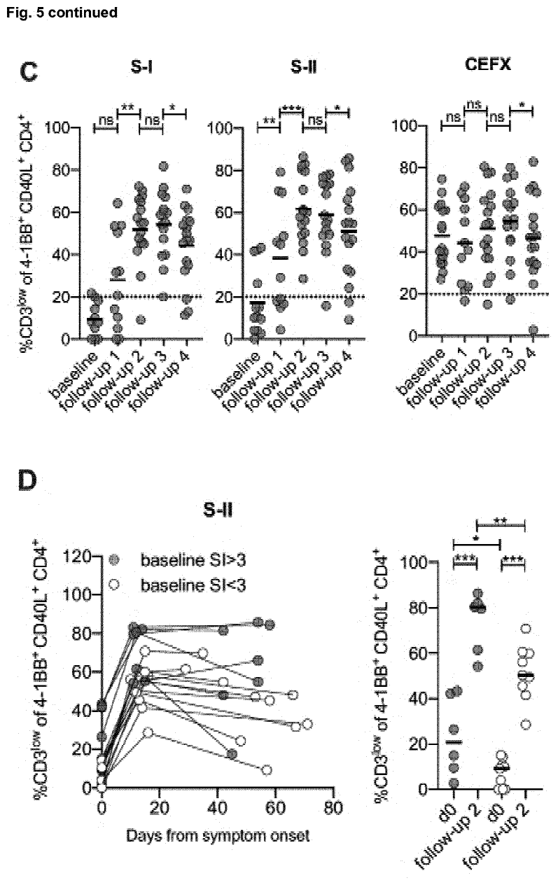

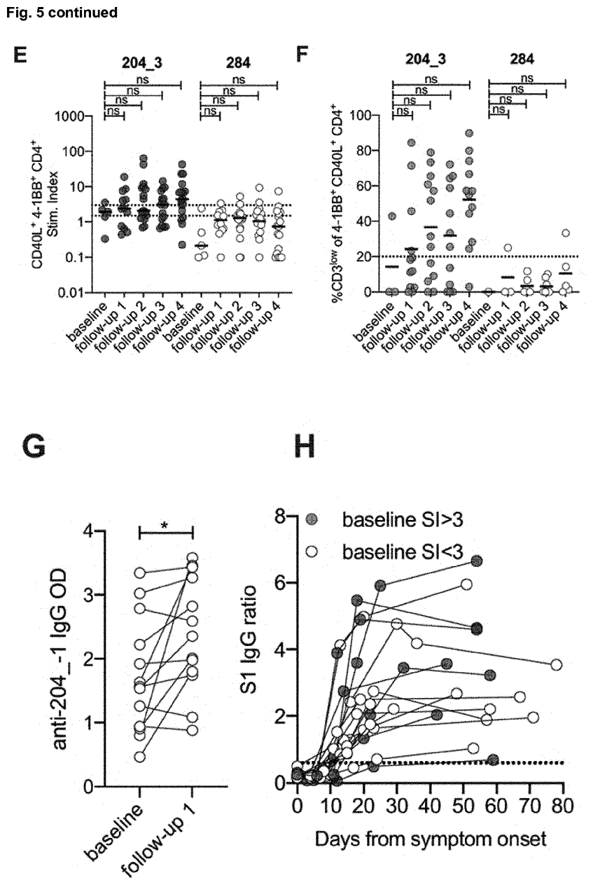

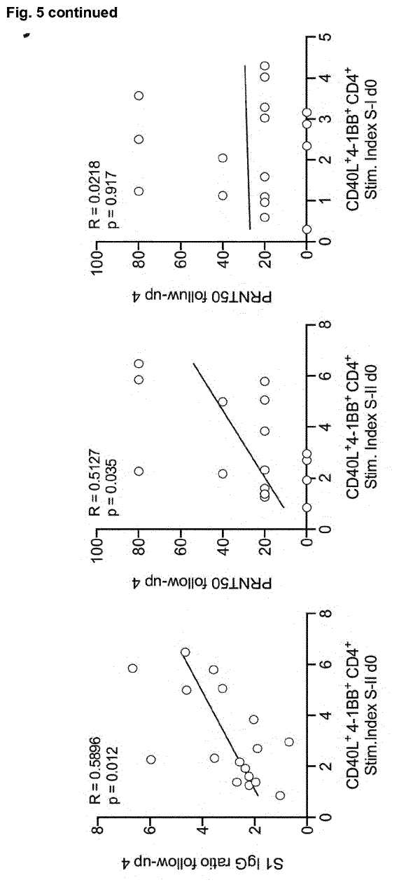

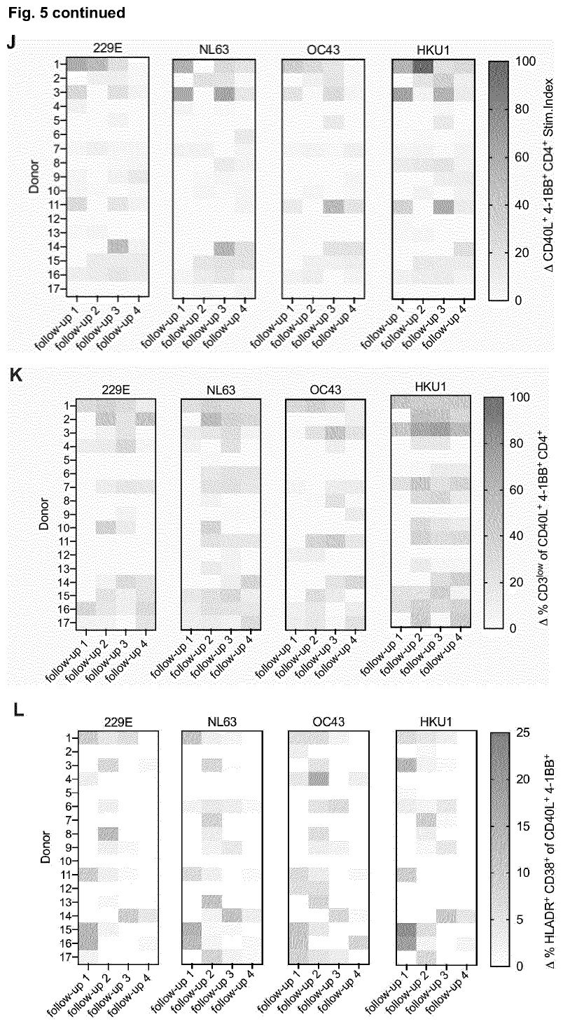

FIG. 5: HCoV-specific SARS-CoV-2-cross-reactive T cells are recruited into the primary SARS-CoV-2 infection response

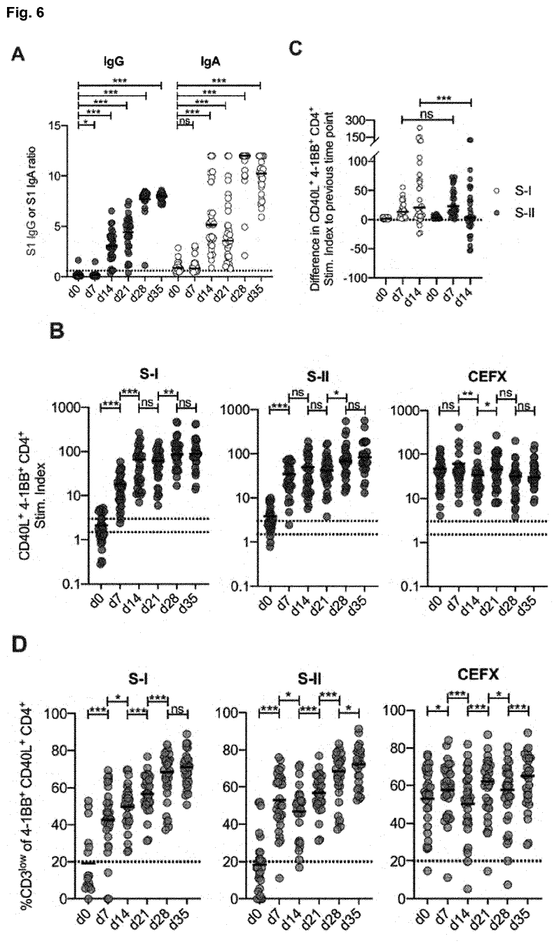

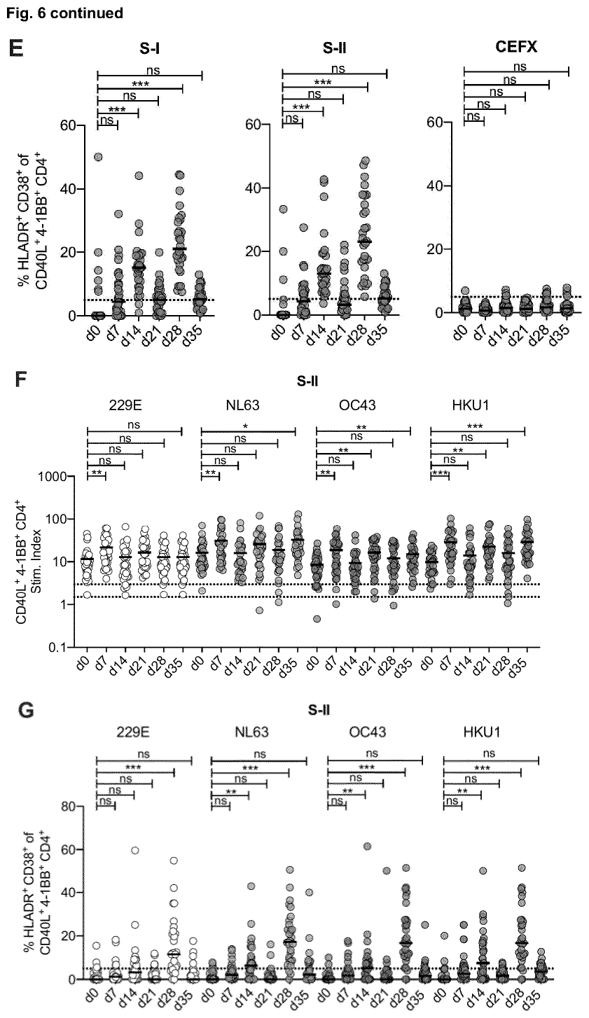

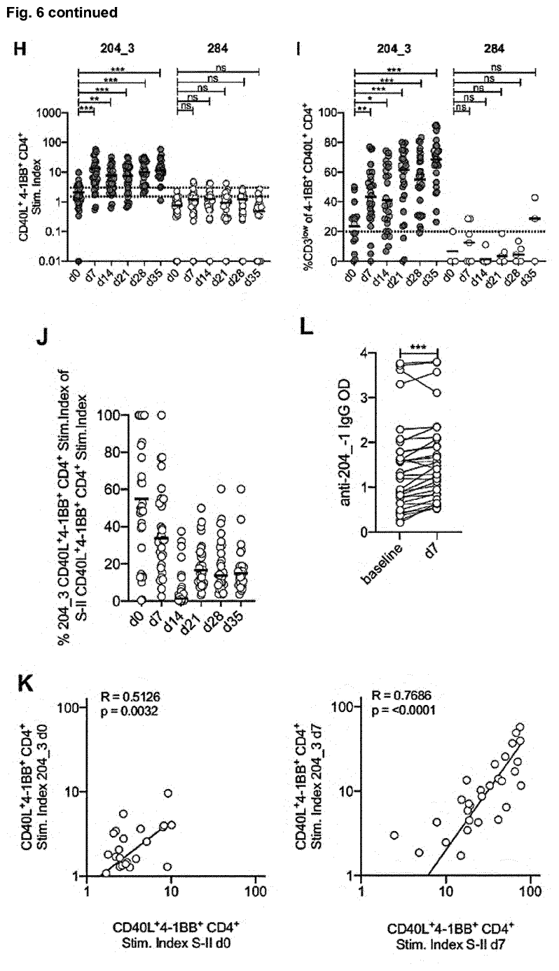

FIG. 6: HCoV-specific SARS-CoV-2-cross-reactive T cells are recruited into the BNT162b2 vaccine response

FIG. 7: HCoV homology scores and SARS-CoV-2 peptide pool reactivity (A) Similarity

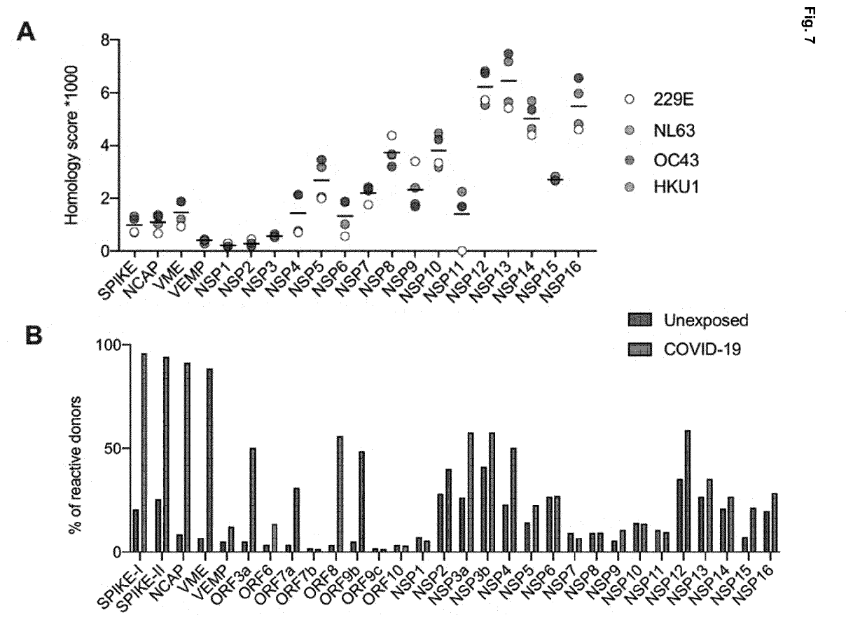

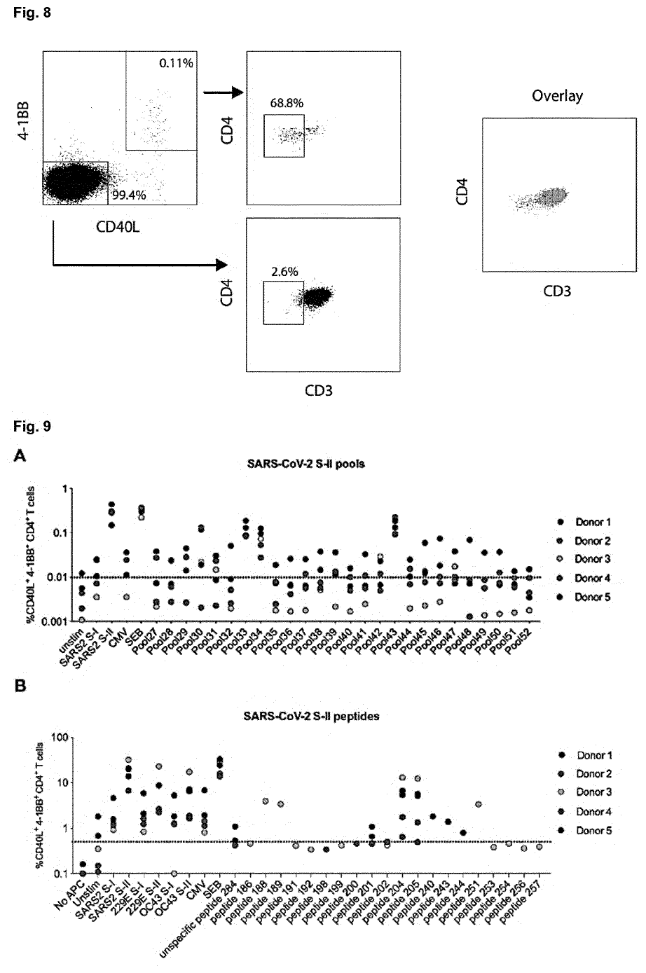

FIG. 8: CD3low gating in flow cytometry

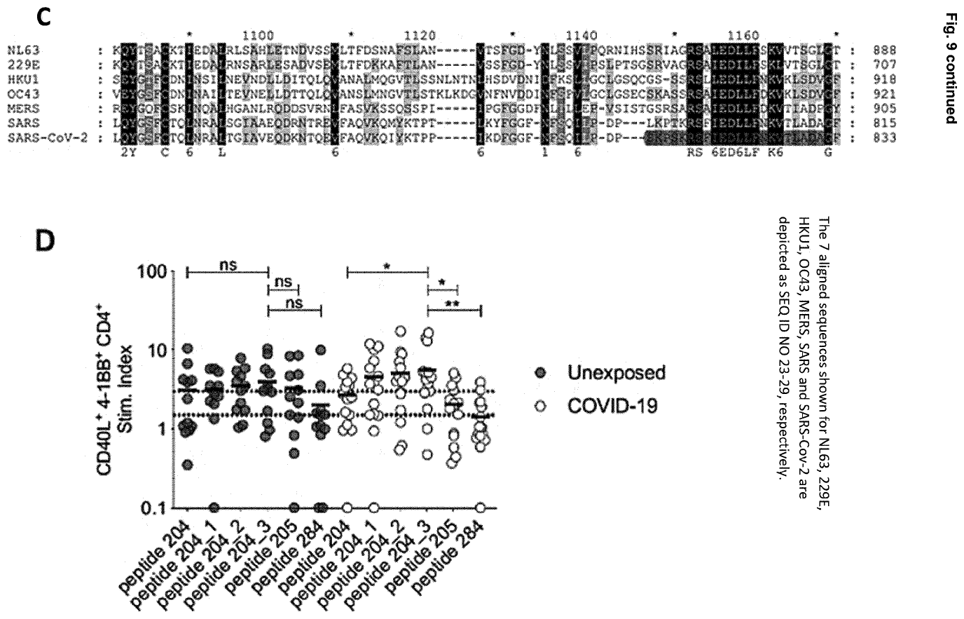

FIG. 9: Identification of iCope by narrowing down the sequence regions mapped in SARS-CoV-2 peptide pools that exhibited highest T cell cross-reactivity.

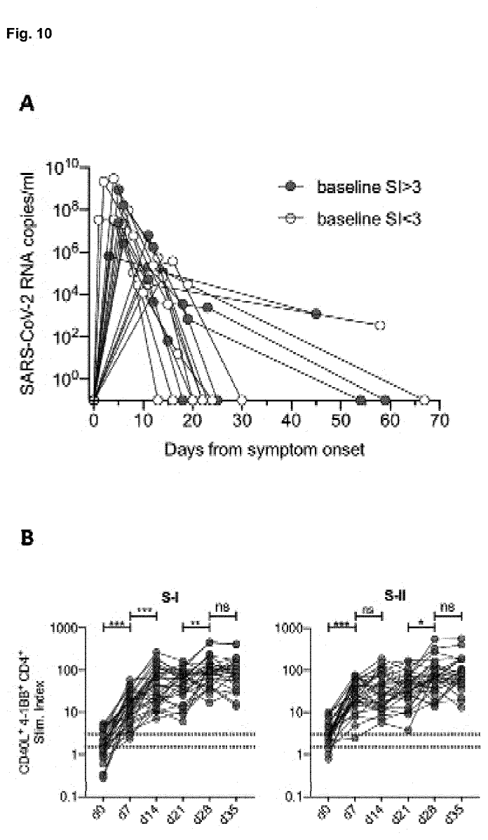

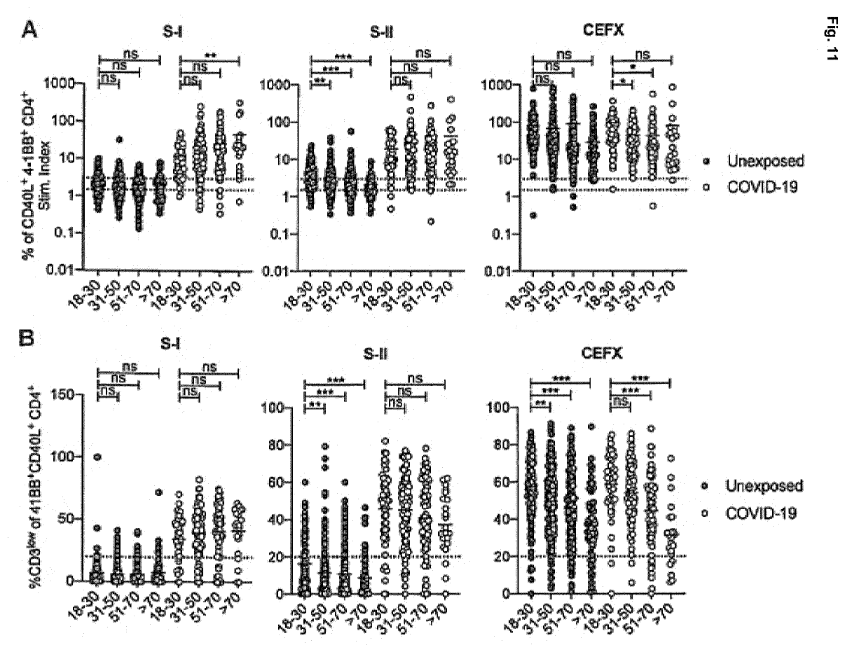

FIG. 10: Virus load during SARS-CoV-2 infection and individual T cell responses to vaccination

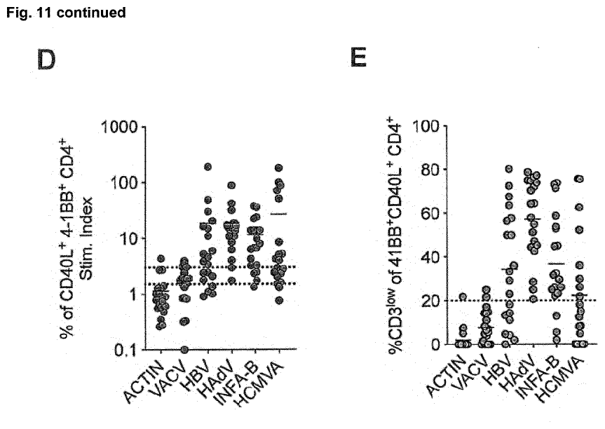

FIG. 11: Direct analysis of functional avidity of CD4+ T cells.

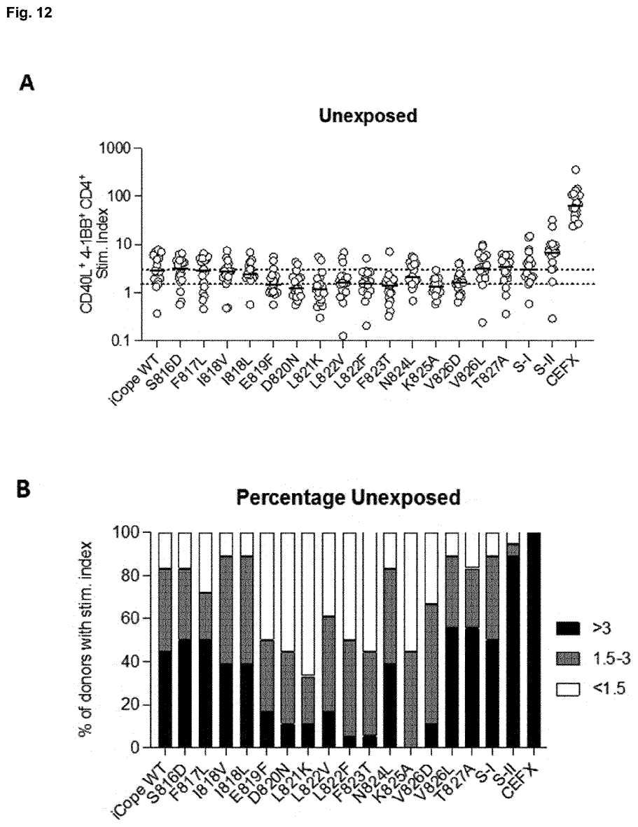

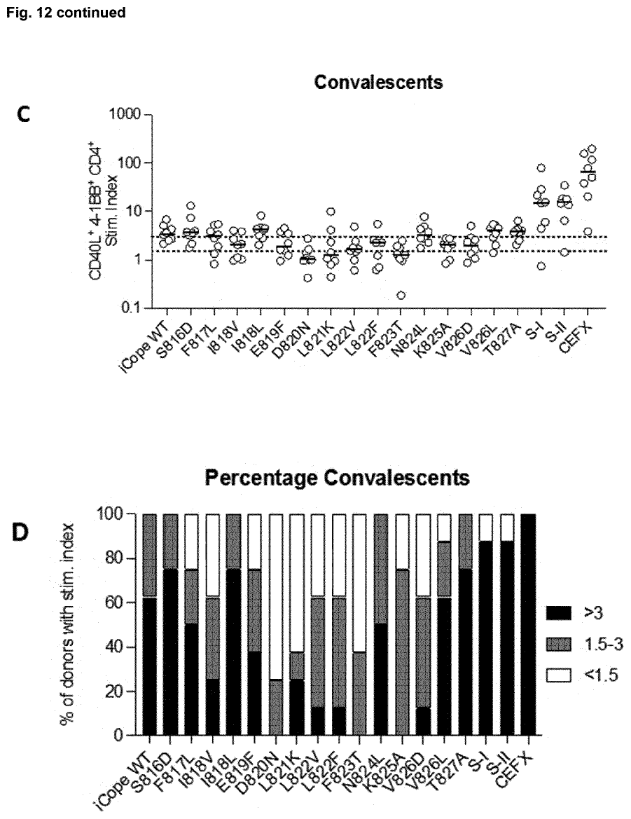

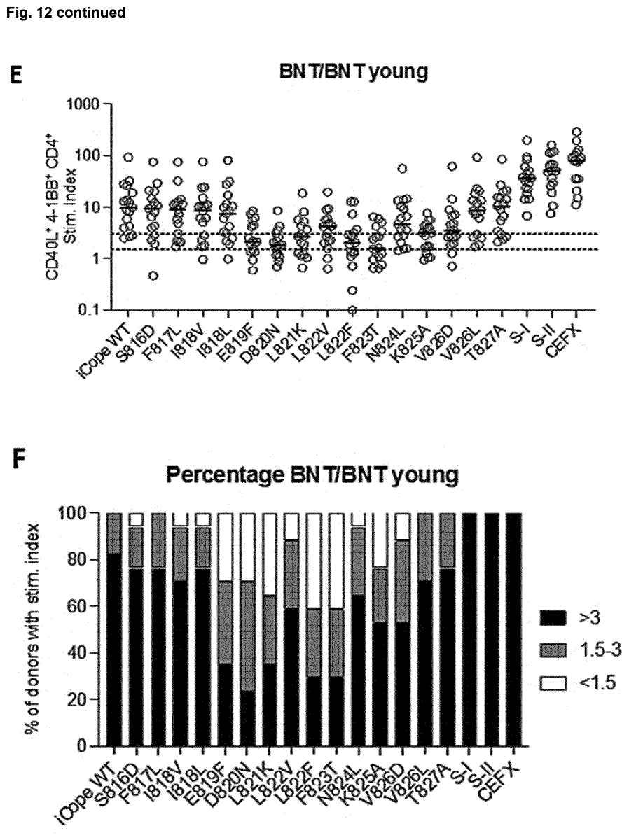

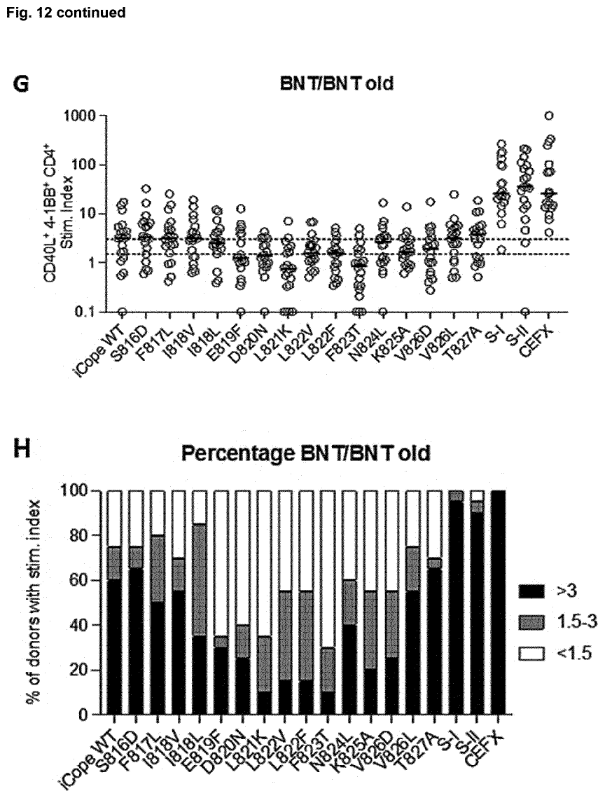

FIG. 12: Mutations in S819-826 impair T cell responsiveness in unexposed, convalescents and in vaccinated subjects

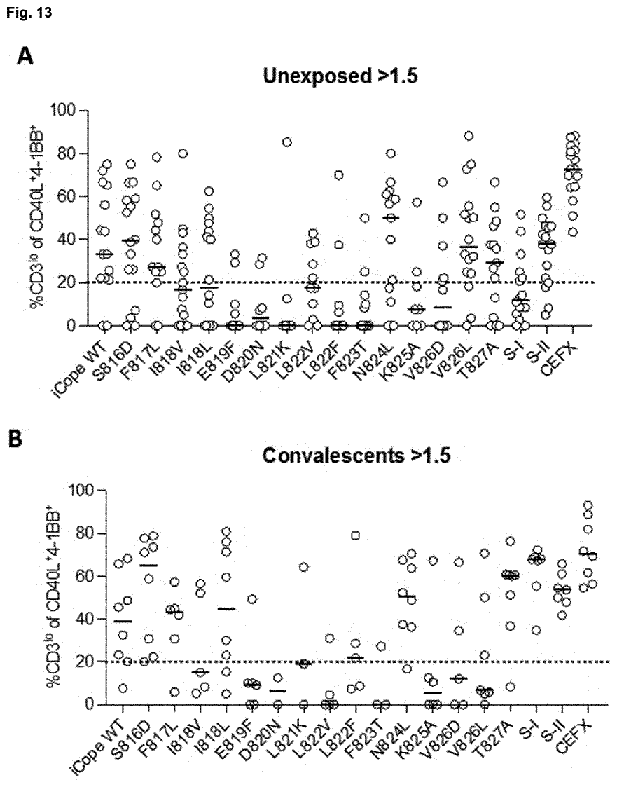

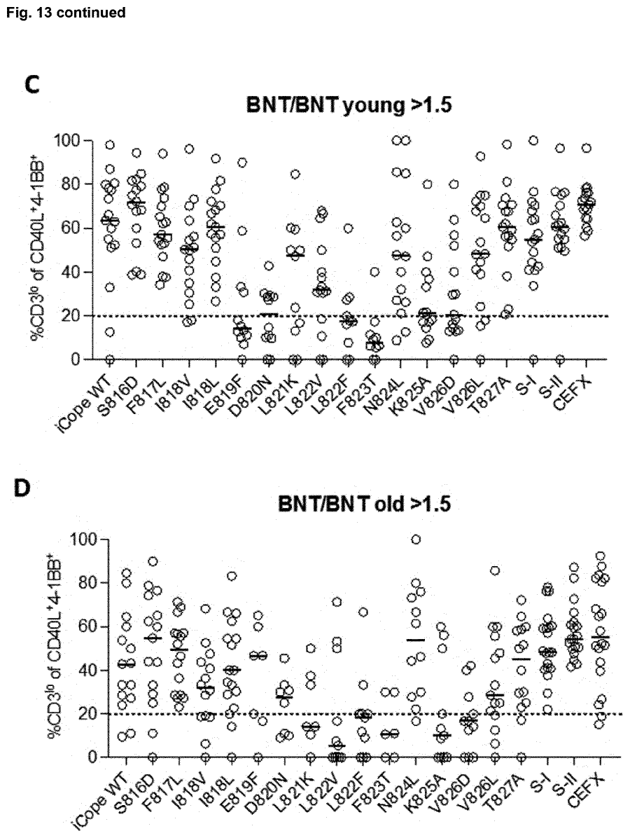

FIG. 13: Mutations in S819-826 impair the quality of iCope responsiveness, especially in the elderly

FIG. 14: Impact on COPE sequence mutation on cytokine response of PBMCs.

### DETAILED DESCRIPTION OF THE FIGURES

FIG. 1: CD4⁺ T cell cross-reactivity against the SARS-CoV-2 orfeome

(A) Stimulation index (stim. index) of reactive CD40L⁺4-1BB⁺ CD4⁺ T cells among PBMCs stimulated with indicated SARS-CoV-2-orfeome peptide pools. Grey labels below indicate proteins exclusive for SARS-CoV-2 (not shared with HCoVs). Grey (COVID-19) or dark red (Unexposed) dots mark donors with an SI≥3. n=60 unexposed, n=59 COVID-19 convalescents. Dotted lines indicate stim. index of 1.5 and 3. *p<0.05, **p<0.01, ***p<0.001 and ns=not significant for p>0.05 (unpaired student's t-test). Results for statistical significance are shown above Unexposed plot. (B) Proportion of individuals with indicated number of SARS-CoV-2-orfeome peptide pool stimulations with a stim. index≥3. (C) Proportion of individuals with stim. index≥3 for each stimulation in each indicated stimulation combination. (D) To determine the probability of homologous epitopes, a homology score for the comparison of a SARS-CoV-2 antigen with all 4 endemic coronaviruses 229E, NL63, OC43 and HKU1 (isolates N1, N2 N5) is plotted against the mean stim. index of 60 unexposed donors (circles) and 59 COVID-19 convalescents (triangles). For the calculation of the homology score all theoretical possible 9mers in each peptide of a respective PepMix were scored against all 9mers of the complete proteome of all other HCoVs using a PAM30 substitution matrix. The homology score is the percentage of comparisons between the 9mers from the PepMix and all 9mers from the HCoVs with a score above 30.

FIG. 2: Magnitude of SARS-CoV-2-cross-reactivity decreases with age

(A) Stim. index of CD40L⁺41 BB⁺ CD4+ T cells after stimulation of PBMCs with SARS-CoV-2 S-I, SARS-CoV-2 S-II, or CEFX (CMV, EBV, Flu and other common pathogens epitope pool) in n=568 unexposed donors and n=174 COVID-19 convalescents divided into age groups. (B, C) Stim. index of CD40L⁺4-1BB⁺ CD4+ T cells and (C) frequencies of CD3^{low} cells among CD40L⁺4-1BB⁺ CD4⁺ T cells after PBMC stimulation with indicated peptide pools. CD3^{low} frequencies are shown for antigen responding donors (stim. index≥1.5) except for ACTIN and VACV where CD3^{low} frequencies are shown for all CD40L⁺4-1BB⁺ T cells as low control. Dotted lines indicate stim. index of 1.5 and 3 (B) or 20% (C). (D) Frequency of CD3^{low} cells among S-I or S-II-reactive CD40L⁺4-1BB⁺ CD4+ T cells in different age groups after stimulation of PBMC from unexposed and COVID-19 convalescents. CD3^{low} frequencies are shown for T cell responses with a stim. index≥1.5.

A: *p<0.05, **p<0.01, ***p<0.001, and ns for p>0.05, One-way-ANOVA with Dunnett correction.

B, C, D: *p<0.05, **p<0.01, ***p<0.001, and ns for p>0.05 (Student's t-test).

FIG. 3: High functional avidity T cells to spike S-II from HCoVs decrease with age

(A) Stim. index of CD40L⁺4-1BB⁺ CD4⁺ T cells in unexposed (n=568) as well as COVID-19 convalescents (n=174) after PBMC stimulation with HCoV (229E, NL63, OC43, HKU1) spike glycoprotein S-I or S-II peptide pools. (B) Frequency of CD3^{low} cells in CD40L⁺4-1BB⁺ CD4⁺ T cells from unexposed and COVID-19 convalescents in age groups after stimulation with S-I and S-II spike glycoprotein pools of HCoVs. CD3^{low} frequencies are shown for T cell responses with a stim. index≥1.5. A, B: *p<0.05, **p<0.01, ***p<0.001 and ns for p>0.05 (One-way-ANOVA with Dunnett correction).

FIG. 4: Peptide 2043 (iCope) constitutes an immunodominant epitope of SARS-CoV-2 T cell (cross)-reactivity

(A) Proportions of unexposed individuals aged below 65 (n=491) and COVID-19 convalescents (n=174) with S-I or S-II specific responses against HCoV and/or SARS-CoV-2 with a stim. index≥3. (B) Short-term T cell lines derived from OC43 S-I and S-II-reactive primary T cells were restimulated with autologous APC in the presence of OC43 or SARS-CoV-2 spike glycoprotein pool S-I and S-II. Stim. index of CD40L⁺4-1BB⁺ CD4⁺ T cells after re-stimulation is shown. Dotted line indicates stim. index of 3. (C) Stim. index of CD40L⁺4-1BB⁺ CD4+ T cells from unexposed or COVID-19 convalescents after stimulation with the single peptide 204_3, the control single peptide 284 or the S-II peptide pool. (D) Anti-204_-1-peptide IgG ELISA. The ELISA plates were coated with a 18mer peptide overlapping 11 amino acids with the 204_3 peptide. Serum from unexposed and elderly (>65) individuals as well as COVID-19 convalescents were used at 1:100 dilution. Optical density (OD) of detected IgG antibodies is shown. (E) Distribution of common HLA alleles in definite iCope responders (stim. index≥3) and definite non-responders (stim. index≤1.5), n=308, +/+=homozygous, +/−=heterozygous.

C, D: *p<0.05, **p<0.01, ***p<0.001 and ns for p>0.05 (Student's t-test).

FIG. 5: HCoV-specific SARS-CoV-2-cross-reactive T cells are recruited into the primary SARS-CoV-2 infection response

(A-C) Stim. index of CD40L⁺4-1BB⁺ CD4⁺ T cells (A), frequencies of HLADR⁺CD38⁺ (B), and frequencies of CD3^{low} (C) in CD40L⁺4-1BB⁺ CD4⁺ T cells after stimulation with SARS-CoV-2 S-I, S-II and CEFX peptide pools of donors prior (baseline) to and at 4 different time points after SARS-CoV-2 infection. CD3^{low} frequencies are shown for T cell responses with a stim. index≥1.5. (D) Overview of changes in CD3^{low} frequency in CD40L⁺4-1BB⁺ CD4⁺ T cells between baseline, follow-up measurement time point 2 (between d10-d16 after symptom onset) and follow-up measurement time point 4 (between d29-d71) (left plot) and statistics (right plot) for baseline and follow-up measurement time point 2 in cross-reactive donors (baseline stim. index 3, red circles) and non-cross-reactive donors (baseline stim. index≤3, white circles). (E) Stim. index of CD40L⁺4-1BB⁺ CD4⁺ T cells and (F) frequency of CD3^{low} of CD40L⁺4-1BB⁺ CD4⁺ T cells after stimulation with peptide 204_3 (iCope) or control peptide 284. CD3^{low} frequencies are shown for T cell responses with a stim. index 21.5. (G) Anti-204_-1-peptide IgG ELISA. The ELISA plates were coated with an 18-mer peptide overlapping 11 amino acids with the 204_3 peptide. Sera from baseline and 7 days after symptom onset were used at a 1:100 dilution. Optical density (OD) of detected IgG antibodies is shown. (H) Kinetics of anti-S1-IgG antibody titer ratios in cross-reactive donors (baseline stim. index≥3, red circles) and non-cross-reactive donors (baseline stim. index≤3, white circles). (1) Correlation of anti-S1-IgG antibody titer ratio at last sampling time point FU4 (29-71 days after symptom onset) to the frequency of CD40L⁺4-1BB⁺ CD4⁺ upon S-II stimulation at d0 (baseline, left plot), and correlation of neutralizing antibody titers PRNT50 at last sampling time point FU 4 to frequency of CD40L⁺4-1BB⁺ CD4⁺ upon stimulation with S-II (middle plot) or to stimulation with S-I at d0 (baseline, right plot). (J, K, L) Heat maps with the delta (A) of stim. index (J), frequency of CD3^{low} (K) and frequency of HLADR⁺CD38⁺ cells (L) of CD40L⁺4-1BB⁺ CD4⁺ T cells after stimulation with S-II pools of indicated HCoVs. Delta is the change of the parameter at the given time point relative to baseline, i.e., white depicts no increase.

A, C, D, F, G: *p<0.05, **p<0.01, ***p<0.001 and ns for p>0.05 (Paired Student's t-test).

B: *p<0.05, **p<0.01, ***p<0.001 and ns for p>0.05 (repeated measure one-way-ANOVA with Dunnett correction).

E: ns for p>0.05 (paired Student's t-test).

I: Pearson correlation.

FIG. 6: HCoV-specific SARS-CoV-2-cross-reactive T cells are recruited into the BNT162b2 vaccine response

(A) Serum IgG and IgA S1 titer ratio at baseline (d0) and d7, d14, and d21 after primary immunization with the BNT162b2 COVID-19 vaccine, as well as d7 (d28) and d14 (d35) after secondary vaccination. Secondary vaccination occurred on day 21. IgA max value set as 12.

(B) Stimulation index of CD40L⁺4-1BB⁺ CD4+ T cells after stimulation with S-I, S-II and CEFX. (C) Difference of stim. index relative to the previous time point measured. (D) Frequency of CD3^{low} of CD40L⁺4-1BB⁺ CD4+ T cells after stimulation with S-I, S-II and CEFX. CD3^{low} frequencies are shown for T cell responses with a stim. index≥1.5. (E) Frequencies of HLADR⁺CD38⁺ of CD40L⁺4-1BB⁺ CD4+ T cells after stimulation with S-I, S-II and CEFX. (F, G) Stim. index (E) and frequency of HLADR⁺CD38^{low} (F) of CD40L⁺4-1BB^{low} CD4⁺ T cells after stimulation with HCoV S-II peptide pools at baseline and indicated days after primary vaccination. (H, I) Stim. index (G) and frequencies of CD3^{low} (H) of CD40L⁺4-1BB⁺ CD4+ T cells after stimulation with peptide 204_3 (iCope) and control peptide 284. CD3^{low} frequencies are shown for T cell responses with a stim. index≥1.5. (J) Ratio of 204_3-reactive T cells to SARS-CoV-2 S-II-reactive T cells. (K) Correlation of stimulation with single peptide 204_3 to stimulation with SARS-CoV-2 S-II peptide pool at d0 or d7, respectively for vaccinees. (L) Anti-204_-1-peptide IgG ELISA. The ELISA plates were coated with an 18-mer peptide overlapping 11 amino acids with the 204_3 peptide. Sera from before and 7 days after primary vaccination were used at a 1:100 dilution. Optical density (OD) of detected IgG antibodies is shown.

A, E, F, G, H, I: *p<0.05, **p<0.01, ***p<0.001 and ns for p>0.05 (repeated measure one-way-ANOVA with Dunnett correction).

B, C, L: *p<0.05, **p<0.01, ***p<0.001 and ns for p>0.05 (Paired Student's t-test).

D: *p<0.05, **p<0.01, ***p<0.001 and ns for p>0.05 (Student's t-test).

K: Pearson correlation.

FIG. 7: HCoV homology scores and SARS-CoV-2 peptide pool reactivity (A) Similarity between SARS-CoV-2 and the different endemic coronaviruses 229E, NL63, OC43 and HKU1 (isolate N1). Scores were calculated using a PAM30 substitution matrix for all 9mers of the respective antigen against all 9mers of the proteome of SARS-CoV-2. The homology score is the percentage of 9mer pairs with a score above 30. (B) Frequency of donors with reactive T cells against indicated peptide pools in unexposed and COVID-19 convalescents from FIG. 1A with a stim. index≥3.

FIG. 8: CD3^{low} gating in flow cytometry

Representative dot plot of gating for CD3^{low} cells within non-reactive CD4⁺ T cells and S-II-reactive CD40L⁺4-1BB⁺ CD4+ T cells. Cells were pre-gated on alive, doublet-free CD4⁺ lymphocytes.

FIG. 9: Identification of iCope by narrowing down the sequence regions mapped in SARS-CoV-2 peptide pools that exhibited highest T cell cross-reactivity.

(A) Expanded SARS-CoV-2 S-I or S-II specific CD40L⁺4-1BB⁺ CD4⁺ short-term-culture T cells were restimulated with autologous APC in the presence of different matrix pools and the frequency of CD40L⁺4-1BB⁺ reactive cells determined. Cut-off line is set at 0.01 based on unstimulated controls. (B) Expanded SARS-CoV-2 S-I or S-II reactive CD40L⁺4-1BB⁺ CD4+ T cells were restimulated with autologous APC in the presence of single peptide candidates determined from matrix stimulation (shown in A) and the frequency CD40L⁺4-1BB⁺ reactive CD4⁺ T cells was determined. Cut-off line is set at 0.5 based on unstimulated controls. (C) Position of single peptides 204 and 205 in sequence overlay of 7 known human coronaviruses. Color code indicates homology: black=identical amino acid, grey=conservative amino acid replacement. (D) Identification of the dominant peptide at the 204 to 205 sequence intersection. The number behind the underline character indicates the number of amino acids by which the 15-mer was shifted towards the C-terminus beginning with the sequence of 204 and ending with the sequence of 205. Only a selection of donors is shown that displayed a stim. index 3 for a minimum of one stimulation.

FIG. 10: Virus load during SARS-CoV-2 infection and individual T cell responses to vaccination
(A) Relative quantities of SARS-CoV-2 as measured by PCR at indicated time points. Donors with stim. index 3 for SARS-CoV-2 S-II at baseline shown as red circles, individuals with stim. index≤3 shown with white circles. (B) Individual vaccinee response kinetics of CD40L⁺4-1BB+CD4⁺ T cells upon stimulation with S-I or S-II, as shown in FIG. 6B. *p<0.05, **p<0.01, ***p<0.001 and ns for p>0.05 (Paired Student's t-test). Secondary vaccination occurred on day 21.

FIG. 11: Direct analysis of functional avidity of CD4+ T cells.
(A) and (B) show experiments with cross-reactivities against SARS-CoV-2 spike glycoprotein S-I and S-II in healthy donors (unexposed) and in COVID-19 convalescents (COVID-19). In addition, control peptide pools composed of peptides that are immunodominant epitopes of various common pathogens (CEFX) were applied here. (D) shows the stimulation indices of CD4+ T cells responding to each antigen pool. (E) shows the % of CD3-low cells within the CD4+ T cells responding to the respective antigen pools.

FIG. 12: Mutations in S819-826 impair T cell responsiveness in unexposed, convalescents and in vaccinated subjects

Ex vivo stimulation of PBMCs from unexposed individual (A,B), COVID-19 convalescent patients (C, D) and BNT/BNT vaccinated individuals (young: E, F; old: G, H) with iCope WT or different mutated iCope peptides and control pools S-I, S-II and CEFX. (A, C, E, G) The percentage of CD40L⁺4-1BB⁺ CD4+ T cells among stimulated PBMC was divided by the percentage of these cells among unstimulated PBMC to determine the stimulation index (stim. index) shown on the y-axis. Dotted lines indicate a stim. index of 1.5 and 3. (B, D, F, H) Bars show the proportions of individuals with the indicated peptide or peptide-pool stimulations with a stim. index>3, 1.5-3 or <1.5. (A, B) Bars show the proportions of individuals with the indicated peptide or peptide-pool stimulations with a stim. index>3, 1.5-3 or <1.5. *P<0.05, **P<0.01, ***P<0.001 and ns for P>0.05 (Student's t test).

FIG. 13: Mutations in S819-826 impair the quality of iCope responsiveness, especially in the elderly

Ex vivo stimulation of PBMCs from young BNT/BNT vaccinated individuals with iCope WT or different mutated iCope peptides and control pools S-I, S-II and CEFX. CD3 downregulation is significantly reduced in mutations of the positions 819-823 and 825 in all measured cohorts. In general, the mutations in the central sequence region of the iCOPE peptide showed reduced T cell binding and in general also displayed in lower TCR affinity. Homologous BNT/BNT vaccination results in the expansion of high affine iCope clones in the young, but not in the elderly.

FIG. 14: Impact on iCOPE sequence mutation on cytokine response of PBMCs

Ex vivo stimulation of PBMCs from young BNT/BNT vaccinated individuals with iCope WT or different mutated iCope peptides and control pools S-I, S-II and CEFX. Shown is the TNFα and/or IFNg positive or negative percentage of cells. The remaining iCope specific T cells in the vaccinated elderly generally display reduced IFNg-driven antiviral responses. Mutations in the iCope sequence also result in less optimal cytokine responses by the remaining responsive T cells.

### EXAMPLES

### Example 1: Method for Direct Analysis of Functional Avidity of CD4+ T Cells

Initially, the assay was directly applied to assess well-characterized immune responses against pathogens and vaccine antigens (in healthy subjects). One group of subjects younger than 30 and a second group older than 70 were studied. The following antigen-peptide pools were used:
ACTIN (control peptide pool with strongly expressed autoantigen, therefore no or weak response expected with low functional avidity).
VACV (vaccinavirus) (peptide pool of cell surface-binding protein MVA, no reactivity in non-vaccinated)
HBV (hepatitis B virus) (peptide pool of HBV vaccine antigen, reactivity and functional avidity according to distance from last vaccination)
INFA-B (influenza) (different reactivities in all individuals)
INFA-H (influenza) (different reactivities in all individuals)

The method, enabling direct analysis of functional quality of immunity, included providing PBMC from donors of both age groups, and stimulating PBMC for 16 h with the respective indicated peptide pools.

Subsequently, cells were fixed and labeled with antibodies against CD4, CD3, CD40L, and 4-1BB, among others. Flow cytometric analysis was performed on a MACSQuant 16 cytometer (semiautomated sample acquisition).

Shown in FIG. 11D are the stimulation indices of CD4+ T cells responding to each antigen pool.

FIG. 11E shows the % of CD3-low cells within the CD4+ T cells responding to the respective antigen pools.

Using analysis of CD3 expression, functional avidities can now be additionally and directly determined. Responses in the range up to about 20% (e.g. as seen for actin) are to be considered as only poorly functional, or as exhibiting low function. Here it can be assumed that recognition by memory T cells occurs with only low functional avidity, i.e. such T cells would for example recognize completely different antigenic peptides via their immune receptors, occurring for example also by chance. Values of >50% indicate T cell reactivities with high functional avidity.

The combined analysis allows here for the first time an overall assessment of T cell reactivities.

We tested this technique directly in another series of experiments with cross-reactivities against SARS-CoV-2 spike glycoprotein S-I and S-II in healthy donors (unexposed) and in COVID-19 convalescents (COVID-19). In addition, control peptide pools composed of peptides that are immunodominant epitopes of various common pathogens (CEFX) were applied here.

Results are shown in FIGS. 11A and 11B.

First, we can demonstrate that the proportions of cross-reactive T cells against SARS-CoV-2 spike glycoprotein S-I remain consistent with age and are low. Slightly higher proportions are observed for cross-reactive T cells against SARS-CoV-2 spike glycoprotein S-II, although these also decrease with age.

Using the present “CD3-low” analysis technique, we can now directly demonstrate for the first time that cross-reactivities in healthy unexposed donors against SARS-CoV-2 spike glycoprotein S-I are of low functional avidity, whereas cross-reactivities against SARS-CoV-2 spike glycoprotein S-II are of higher functional avidity. However, the proportion of such cells with high functional avidity decreases with age. This decrease in cells with high functional avidity is also observed in CEFX reactive T cells.

Applications of the Invention:

Various applications are conceivable in which, for the first time, critical conclusions can be drawn about the quality of immune responses in malignancies, in infections, and in the status of and/or recovery from various disease states. Immune responses can be characterized not only functionally in terms of distinct messengers or activation markers, but also in terms of their functional avidity. Poor functional avidity will generally be associated with a non-efficient (because, for example, too slow) immune response. In the case of infections, this means that the pathogen may multiply more rapidly (as in e.g. SARS CoV 2) and then infect cells or tissues systemically. Thus, severe damage occurs.

### Example 2: Cross-Reactive CD4⁺ T Cells Enhance SARS-CoV-2 Immune Responses Upon Infection and Vaccination

Frequent and Broad SARS-CoV-2-Cross-Reactivity in Unexposed Healthy Donors

To determine the extent of cellular cross-reactivity, we stimulated CD4⁺ T cells of 60 unexposed healthy donors and 59 COVID-19 convalescents as controls with the complete SARS-CoV-2 orfeome (FIG. 1A). The SARS-CoV-2 orfeome consists of 11 ORFs (NCBI), five of which (ORF1a/b, NCAP, spike, VEMP and VME) are also found in HCoVs 229E, OC43, NL63 and HKU1, with ORF1a/b coding for a total of 16 non-structural proteins (NSPs). Sequence alignment revealed discrete stretches of high homology in almost all SARS-CoV-2 proteins to the corresponding proteins in HCoVs. Parts of the ORF1a/b including NSP8, NSP10 and NSP12-16 displayed the highest frequencies of homology and thus potential cross-reactive epitopes to all HCoVs (FIG. S7A). COVID-19 convalescents displayed CD4⁺ T cells reacting strongly against spike N-terminal S-I (amino acid residues 1-643) and C-terminal S-II (amino acid residues 633-1273), NCAP, and VME peptide pools (FIG. 1A, B). However, there was no increased reactivity among convalescents compared to SARS-CoV-2-unexposed individuals with respect to NSP8-16, some of which showed the highest degree of sequence homology between HCoVs and SARS-CoV-2. Reactivity against the combination of S-I, S-II, NCAP, and VME peptide pools clearly distinguished unexposed individuals from COVID-19 convalescents irrespective of the disease course (FIG. 1C). In unexposed individuals, we detected low CD4+ T cell reactivity against virtually all SARS-CoV-2 antigens to various extent, including those exclusive to SARS-CoV-2 (FIG. 7B), however, there was no obvious correlation between homology and cross-reactivity across the proteins (FIG. 1D). Thus, we identified cognate cross-reactivity likely to result from previous exposure to similar proteins found in HCoVs with respect to, for example, the spike protein, but also some non-cognate cross-reactivity, i.e. cross-reactivity that cannot be explained by the previous exposure to similar proteins in HCoVs. Of all 30 orfeome peptide pools, spike S-I/-II was the only one that elicited T-cell reactivity in all COVID-19 convalescents and additionally responses in some of the unexposed individuals. In addition, antibodies to spike induced by SARS-CoV-2 infection can neutralize the virus (18), and most of the recently approved and highly effective SARS-CoV-2 vaccines are also based on spike. Therefore, we next assessed pre-existing SARS-CoV-2 spike-cross-reactive T cells in unexposed individuals in more detail.

SARS-CoV-2 Spike S-II-Cross-Reactive T Cells Decrease with Age

A striking feature of SARS-CoV-2 infection is the high correlation of age with disease severity. Immunologically, aging is associated with a lack of newly generated T cells and clonal expansion of few clones as a result of persistent infections, which limits the breadth and quality of T cell responsiveness (19). In order to assess the effect of age on SARS-CoV-2-(cross)-reactive T cell immunity we first examined the breadth of SARS-CoV-2 spike-specific CD4⁺ T cell responses in 568 unexposed individuals and 174 COVID-19 convalescents from different age groups (FIG. 2A). COVID-19 convalescents displayed a statistically significant age-associated increase in spike S-I reactive T cells that correlated with higher disease severity in the older cohorts. However, in line with our previous findings (3) in unexposed individuals, T cell cross-reactivity to the N-terminal portion of spike (S-I) was rare, close to the limit of detection, and remained at low levels with increasing age, whereas reactivity to the C-terminal portion of spike (S-II) was more frequent and higher but decreased with increasing age. T cells reacting to a peptide pool representing a mixture of selected T cell epitopes from common pathogens (CEFX pool), by contrast, remained stable with age (FIG. 2A). Thus, elderly individuals exhibited decreased cross-reactivity immunity to the more HCoV-homologous spike S-II portion.

Low CD3 Surface Expression Identifies SARS-CoV-2 Reactive T Cells with High Functional Avidity Ex Vivo

To assess the quality of the spike-(cross-)reactive T cell response in terms of functional T cell avidity, we examined the level of CD3 surface expression in CD40L⁺4-1BB⁺ CD4+ T cells induced upon short-term in vitro stimulation. Strong T cell receptor (TCR) activation, characteristic of T cells with high TCR avidity, blocks recycling of the TCR:CD3 complex and hence is detectable by reduction in CD3 surface expression (20), hereafter referred to as high functional avidity. Thus, cognate cross-reactivity with higher probability of high functional avidity should be distinguishable from non-cognate cross-reactivity with higher probability of low functional avidity by analyzing the frequency of CD3^{low} T cells among CD4+ T cells activated via their TCR. Accordingly, when stimulated with the peptide pools of the autoantigen ACTIN or vaccinia virus (VACV, unvaccinated donors, FIG. 2B), the few reactive T cells exhibited only low non-cognate reactivity, characterized by low antigen avidity with CD3^{low} frequencies below 20% (FIG. C, FIG. 8). By contrast, T cell responses to the hepatitis B vaccine as well as to recurrent infections (Influenza A, Adenovirus and CMV, FIG. 2B) were characterized by high frequencies of reactive T cells and high frequencies of high avidity CD3^{low} cells (FIG. 2C). After stimulations with spike S-I/S-II peptide pools, COVID-19 convalescents showed high frequencies of spike S-I and S-II-activated CD4⁺ T cells that largely lacked CD3 expression (FIG. 2D). In unexposed individuals, the frequency of CD3^{low} cells among spike S-I and S-II-activated CD4+ T cells was markedly lower, but significantly higher frequencies of CD3^{low} cells were present among S-II- than among S-I-activated CD4⁺ T cells across all age groups (FIG. 2D). These results are consistent with the high homology of the C-terminal S-II portion of SARS-CoV-2 spike to the analogous S-II portion of HCoV spike. These results indicate that spike S-II (cross)-reactive CD4⁺ T cells comprise cells with high functional avidity.

HCoV Spike-Reactive High Functional Avidity CD4⁺ T Cells Decreases with Age

We hypothesized that previous HCoV exposures induce cognate cross-reactive CD4⁺ T cells. Therefore, we next characterized CD4⁺ T cell immunity to HCoV spike in unexposed individuals and COVID-19 convalescents, again grouped by age. HCoV-S-I and S-II-reactive CD4+ T cells were more readily detectable than SARS-CoV-2 spike-specific T-cells and found in 80% (S-I) and 98% (S-II) of SARS-CoV-2 unexposed individuals, respectively (FIG. 3A). However, their frequency significantly decreased with age, and in this instance this reduction was also detectable for the S-I-reactive T cells. Aggregated as a group, SARS-CoV-2 infection overall did not result in higher HCoV-S-I-/S-II-reactive T cell frequencies in COVID-19 convalescents compared to unexposed individuals. HCoV-reactive T cells were also found to decrease as a function of age. We also examined the functional avidities of HCoV-reactive CD4⁺ T cells (FIG. 3B). Among HCoV spike S-I- and S-II-reactive CD4⁺ T cells, high frequencies of CD3^{low} T cells were found, which again significantly decreased with increasing age. These results suggest a high degree of HCoV exposure of the population, leading to equally widespread cross-immunity to SARS-CoV-2 spike. HCoV-reactive CD4+ T cells frequently comprised cells with high functional avidity but significantly decreased with age.

Identification of the Immunodominant Peptide “iCope” Recognized by SARS-CoV-2 Spike Glycoprotein S-II-Cross-Reactive CD4⁺ T Cells

All SARS-CoV-2-cross-reactive unexposed donors showed a response against at least 2 (S-I) or 3 (S-II) HCoVs, suggesting that repeated infection with different HCoVs either establishes a detectable prominent SARS-CoV-2 cross-reactive T cell pool and/or specific T cells are directed against highly homologous sequences shared by multiple HCoVs and SARS-CoV-2 (FIG. 4A). We examined next, whether HCoV spike glycoprotein-specific T cells directly cross-react to SARS-CoV-2 spike glycoprotein. Therefore, CD40L⁺4-1BB⁺ OC43 S-I or S-II-reactive CD4⁺ T cells were isolated, expanded in vitro and restimulated with autologous APC in the presence of OC43- or SARS-CoV-2 spike pool S-I and S-II, respectively. 6 out of 18 OC43 S-II specific short-term T cell lines displayed cross-reactivity against SARS-CoV-2 S-II (FIG. 4B) whereas OC43 S-I specific T cell lines lacked cross-reactivity against SARS-CoV-2 S-I. Next, we aimed to identify the underlying SARS-CoV-2 S-II cross-reactive epitopes. To this end, we isolated SARS-CoV-2 S-II-cross-reactive CD4⁺ T cells from 5 unexposed individuals at high purity, expanded the cells in vitro, and examined their specificity upon restimulation with peptide matrices. We identified two T-cell stimulating peptides (peptides 204 and 205) within S-II in all 5 donors (FIG. 9A). Restimulation of the expanded T cell lines with single peptides and negative control peptides validated the specificity for peptides 204 (N′-SKRSFIEDLLFNKVT-C′ (SEQ ID NO 9)) and 205 (N′-FIEDLLFNKVTLADA-C′ (SEQ ID NO 10)) (FIG. 9B). Only one donor responded to some of the other identified candidates (peptides 188, 189, 251; FIG. 9B). Sequence alignment revealed that S-II peptides 204 and 205 were located in a region of spike characterized by high homology with HCoV (FIG. 9C). The peptides 204 and 205 largely overlapped and differed in only 3 amino acids (aa). By designing 15 aa peptides covering the intersection of both peptides we identified the fusion peptide domain 1 sequence N′-SFIEDLLFNKVTLAD-C′ (aa 816-830; (SEQ ID NO 2)) as the immunodominant Coronavirus peptide epitope hereafter referred to as “iCope” (peptide 204_3, FIG. 9D).

We next examined direct ex vivo T cell reactivity against iCope compared to a control peptide 284 (aa 1133-1147) and the SARS-CoV-2 spike S-II peptide pool in 48 unexposed individuals and 22 COVID-19 convalescents. Note that iCope-reactive CD4⁺ T cells were detected in 50% of convalescents and 20% of unexposed individuals with significantly higher frequency in the former (FIG. 4C). Antibodies to the SARS-CoV-2 spike amino acid residues 815-823 (peptide 204_-1) were previously reported in COVID-19 patients but also in unexposed individuals (21). By examining sera from iCope-T-cell-assay-responders and non-responders we detected 204_-1 binding antibodies in all individuals, however, significantly higher concentration of these antibodies was found in COVID-19 convalescents with substantially more iCope-reactive T cells (FIG. 4D). HLA-typing of n=308 unexposed study participants revealed that among the most frequent alleles (found in >10% of individuals in the study) definite iCope responders (stim. index≥3) compared to definite non-responders (stim. index≤1.5) were more frequently positive for HLA-DPB1*02:01, HLA-DPB1*04:02 and, especially homozygous expression of HLA-DPB1*04:01 (FIG. 4E). Since HLA-DPA1*01:03 was found in 100% of the responders and 94.8% of the non-responders we investigated if combinations of HLA-DPA1*01:03 and HLA-DPB1*02:01/DPB1*04:01/DPB1*04:02 were likely to present iCope or fragments thereof. HLA-peptide-binding predictions (www.IEDB.org) identified excellent potential binders which was also true for the homologous peptide of iCope in other HCoVs.

Pre-Existing SARS-CoV-2 S-II-Cross-Reactive T Cells are Recruited into the Immune Response to Primary SARS-CoV-2 Infection

One of the still outstanding questions is to what extent and how SARS-CoV-2-cross-reactive T cells influence the disease course of primary SARS-CoV-2 infection. Therefore, the Charité Corona Cross study recruited 760 healthy unexposed individuals from July 2020 to February 2021 with the objective to recruit them again in case of diagnosed primary SARS-CoV-2 infection. Baseline immunity to spike S-I and S-II of SARS-CoV-2 and all 4 HCoVs was determined in each subject at the first visit. Study participants informed us in the event of symptoms suggestive of SARS-CoV-2 infection, after which multiplex RT-PCR screening for SARS-CoV-2 and all 4 HCoVs was performed. We aimed to re-sample patients on day (d) 7, d14, d21 and >d35 after the assumed infection day by estimating the infection day to be three days before symptom onset. 17 cases of acute primary SARS-CoV-2 infection were identified (FIG. 5). All 17 patients showed mild COVID-19 disease progression and immediately detectable viral titers (FIG. 10). Robust CD4⁺ T cell responses specific of SARS-CoV-2 spike S-I and S-II were detected and the proportions of HLADR⁺CD38⁺ cells among CD40L⁺4-1BB⁺ CD4⁺ T cells significantly increased on follow up time points 1 and 2 (3-16 days) indicating their in vivo activation (FIG. 5A, B). High functional avidity CD3^{low} cells substantially increased during acute primary SARS-CoV-2 infection and remained at a high level when the infection had resolved (FIG. 5C). Individuals who already had spike S-II-cross-reactive CD4⁺ T cells with a stimulation index above 3 at baseline showed superior high functional avidities throughout the initiation of the T cell response (FIG. 5D). iCope-reactive T cells increased in frequency and in functional avidity in 10 of 17 donors after infection (FIG. 5E, F). Notably, IgG antibodies against the 204_-1 peptide were boosted as early as 3-9 days (follow-up 1) after the presumed infection (FIG. 5G). The appearance kinetics and levels of anti-S1-IgG serum antibodies varied widely, being first detectable at follow-up time point 2 and peaking after day 20 in most subjects (FIG. 5H). Anti-S1-IgG ratios at late time points positively correlated with S-II- but not S-I-cross-reactive T cell levels at d0 suggesting that pre-existing cross-reactive CD4⁺ T cells enhanced SARS-CoV-2-specific humoral immunity (FIG. 5I, left). Moreover, the titer of neutralizing antibodies also positively correlated with S-II- but not S-I-cross-reactive CD4⁺ T cells at baseline, indicating a role of cross-reactive CD4⁺ T cells in protection (FIG. 5I, middle and right). Finally, the frequency of pre-existing HCoV-reactive CD4+ T cells increased in almost all individuals shortly after primary SARS-CoV2 infection (FIG. 5J). There was also a concomitant increase in the frequency of CD3^{low} cells (FIG. 5K) and HLADR⁺CD38⁺ cells (FIG. 5L) among HCoV-reactive T cells, demonstrating that pre-existing HCoV-reactive cellular immunity became activated and expanded during primary SARS-CoV-2 infection. Overall, these results indicate that pre-existing SARS-CoV-2 S-II-cross-reactive T cells were recruited into primary SARS-CoV-2 immune responses in healthy unexposed individuals. The quantity and functional avidity of pre-existing cross-reactive cellular immunity corresponded to the quality and magnitude of specific cellular and humoral anti-SARS-CoV-2 responses and thus may efficiently support mild COVID-19 disease courses.

BNT162b2 Vaccination Re-Activates Pre-Existing SARS-CoV-2 Spike S-II-Cross-Reactive T Cells

Finally, we investigated how pre-existing SARS-CoV-2 S-II-cross-reactive T cells in healthy unexposed individuals influenced the course of BNT162b2 COVID-19 spike mRNA vaccine responses. We monitored baseline- and follow up humoral- and T cell responses against SARS-CoV-2- and HCoV spike glycoproteins in 31 healthy adults who underwent primary and booster vaccination with BNT162b2 at d0 and d21. At day 21, 30 of 31 donors had seroconverted for anti-S1-IgG and all donors had seroconverted for anti-S1-IgA (FIG. 6A) with booster vaccination further increasing the antibody levels. BNT162b2 vaccination induced robust S-I and S-II CD4+ T cell responses in all individuals after primary vaccination which were again slightly enhanced by booster vaccination (FIG. 6B). By contrast, the functional avidity of CD40L⁺4-1BB⁺ CD4⁺ T cells was again substantially increased by booster vaccination (FIG. 6D). Moreover, at day 14 all but three donors had high frequencies of HLADR⁺CD38⁺ cells among S-I- and S-II-reactive CD4⁺ T cells indicating their recent in vivo activation (FIG. 6E). Of note, kinetics of S-I- and S-II-reactive T cells differed in that S-II-reactive T cells showed a sharp increase from baseline to 7 days after vaccination but not thereafter, whereas S-I-reactive T cells again showed a marked increase from day 7 to day 14 (FIG. 6B, C), which is indicative of secondary response kinetics of the former and primary response kinetics of the latter (22). Frequencies of HCoV S-II-reactive T cells were significantly increased 7 days after primary vaccination (FIG. 6F), which was in accordance with increased frequencies of HCoV S-II-reactive HLADR⁺CD38⁺ T cells (FIG. 6G), demonstrating that cognate cross-reactive T cells were activated in response to SARS-CoV-2 spike-specific vaccination. All but 2 of 31 donors (94%) responded with high functional avidity T cells to iCope at day 7 and day 14 (FIG. 6H, I). We observed a correlation between the iCope-reactive T cell response and the S-II-reactive T cell response at d0 that was even more pronounced in the early stage of the immune response on day 7 emphasizing the importance of iCope in the anti-SARS-CoV-2 cellular immune response (FIG. 6K). iCope-specific T cells initially contributed to up to 100% of the CD40L⁺4-1BB⁺ cells in S-II stimulations but decreased in proportion as other specificities increased during the course of the immune response (FIG. 6L). Finally, comparable to the cellular response, a humoral response to the iCope-overlapping sequence 204_-1 was detectable upon vaccination as early as 7 days after primary vaccination (FIG. 6M) supporting the notion of secondary response kinetics (23) for pre-existing cross-reactive immunity.

### Discussion

A better understanding of how cross-reactive immunity influences COVID-19 disease severity and virus clearance is central to containment efforts as well as to the evaluation of the most efficient vaccination strategies. Non-cognate cross-reactivity has been reported but plays a minor role compared to HCoV-mediated cognate cross-reactivity (16,23). However, the clinical relevance of pre-existing cognate cross-immunity to SARS-CoV-2 has been a subject of much debate. A recent HCoV infection has been associated with less severe COVID-19 (17). Interestingly, more than 90% of the population is seropositive for at least one HCoV, thus a large proportion of the population might have some underlying humoral immunity (24, 25). However, in pre-pandemic sera from PCR-validated HCoV-positive individuals, cross-neutralization between HCoVs but no neutralizing antibodies to SARS-CoV-2 were found (24). In a further report, only low spike-specific cross-reactive antibody activity was detected in 5 of 34 donors with recent HCoV infection and in 1 of 31 donors without recent HCoV infection, indicating low level and fast decay of humoral cross-immunity (12). Finally, infection with SARS-CoV-2 increased the prevalence of antibodies against seasonal HCoVs which, however, did not provide protection against SARS-CoV-2, thus drawing attention to the role of cross-reactive cellular immunity (9, 24, 25).

Recently, cross-reactive T cells in unexposed individuals for several SARS-CoV-2 antigens utilizing predicted epitopes (4, 5) or megapools of selected peptides were identified (8, 26). Although the 21 kb long ORF1a/b accounts for the majority of the total 29.8 kb coding sequence of SARS-CoV-2, the totality of T cell responses to ORF1a/b encoded proteins in COVID-19 convalescents compared to cross-reactive T-cell responses in healthy unexposed donors had not been previously analyzed. Our work revealed significant cross-reactivity regarding ORF1a/b encoded proteins but also that most of the anti-SARS-CoV-2 reactivity was directed against spike, nucleocapsid and membrane protein. We further demonstrated that the magnitude and quality of SARS-CoV-2 cross-reactivity and HCoV-reactivity declined with age. This failure of the aging immune system to maintain HCoV-induced SARS-CoV-2 cross-reactive T cells might explain, to an extent, the significant increase in life-threatening COVID-19 disease in the older population. Our results demonstrate not only that HCoV-specific, cross-reactive T cells contributed to SARS-CoV-2 immune responses upon infection and vaccination, but also, that cognate cross-reactivity correlates with a slightly faster high quality cellular as well as enhanced humoral response. Mechanistic insights into this observation can be derived from prime-boost experiments in mice. Here, sequential administration of two antigens sharing the same carrier induces pre-existing T-cell help leading to more efficient B-cell recruitment in secondary immunization (27). Further studies in mice showed that T cell signals early in the immune response can be limiting and that after viral infection, the presence of cognate T cell help promotes germinal center formation which is required for high affinity antibody generation (27-29). As early induction of SARS-CoV-2 T cell reactivity has been associated with rapid viral clearance and mild disease (30), cross-reactive T cells that enhance the immune response to SARS-CoV-2 may be a correlate of immune protection, as recently discussed (31, 32).

Upon BNT162b2 vaccination we observed immune responses that exceeded the response to SARS-CoV-2 infection in terms of T cell and antibody levels. Responses to S-II, unlike responses to the non-cross-reactive S-I, displayed a kinetics profile reminiscent of a secondary rather than a primary response (22, 23). These observations may provide an explanation for the results of current large on-going studies showing protection levels against SARS-CoV-2 infection in excess of 75% as early as 15-28 days after primary vaccination with BNT162b2 (33). In addition, just one dose of the BNT162b2 or the Astra Zeneca ChAdOx1 vaccine reduced the risk of hospitalization by 85% and 94%, respectively, on days 28-34 post primary vaccination, indicating markedly high efficiency (34). Interestingly, a single shot vaccination protocol based on A26 adenovirus encoded, modified spike protein from Johnson & Johnson with a reported vaccine efficiency of 66% was recently approved by the FDA and the EMA (Janssen Ad26.COV2.S (COVID-19) Vaccine VRBPAC Briefing document, FDA). Our data supports the notion that cross-immunity induced by HCoV infections may favor milder SARS-CoV-2 infection courses and furthermore provide an immunological explanation for the high efficacy of SARS-CoV-2 vaccines after just one single dose. The known abundance of HCoV infections would imply that subjects with no or reduced immunity to HCoVs would be at a higher risk of failing to control early virus replication and, as a result, provide conditions favoring viral dissemination. In unexposed subjects with sufficient levels of HCoV reactivity and thus pre-existing cross-reactivity against SARS-CoV-2, a single dose of a spike protein based vaccine may provide efficient protection. However, in older people with waning HCoV reactivity and SARS-CoV-2 cross-reactivity, repeated vaccinations may be critical (35).

The immunodominant cross-reactive peptide (iCope) that we identified here is located within the highly conserved spike fusion domain downstream of the S2′ cleavage site (36). We demonstrate that iCope-reactive T cells were efficiently recruited into the SARS-CoV-2 response in the majority of infected and almost all vaccinated individuals. The literature reports variable but generally high frequencies of HLA-DPA1*01:03, DPB1*02:01, DPB1*04:01, and DPB1*04:02 in different populations around the world (allelefrequencies.net). The high allele frequencies of HLA-DPA1*01:03 and, in particular, HLA-DPB1*04:01 as well as increased homozygosity among responders in our population may explain why so many individuals were able to raise a T-cell response to iCope and its homologues in other HCoVs. Of note, IgG screening against spike and nucleocapsid specific epitopes revealed high responses in COVID-19 convalescents as well as multiple cross-reactive responses in healthy donors for a sequence containing the SARS-CoV-2 B cell epitope SFIEDLLF (21) (SEQ ID NO 18) present in iCope and overlapping 73% with iCope. SARS-CoV-2 infection and vaccination with BNT162b2 induced specific antibody production towards this region. Recently, it has been reported that in current vaccine trials, antibodies specific to the S2 portion of spike, in particular, may be involved in the early induction of protection (25, 37). iCope might serve as a universal conserved coronavirus target for both B cells and T cells. Enhancing the immune response to iCope may have protective effects without the possibility of immune evasion by human coronaviruses and should be a focus of future studies.

### Example 3

The inventors showed that the T cell response to a SARS-COVID-19 infection is dominated by the immunodominant coronavirus peptide (iCOPE) sequence of the spike protein of COVID-19. So far, no variant of concern (VOC) from alpha to omicron is mutated in the iCope sequence. However, in view of the apparent importance of the iCOPE sequence for at least the T cell response against COVID-19 the inventors aimed to analyzed the effect of potential future arising mutations Hence, in this example the inventors investigated the impact of existing and potential mutations on cellular and humoral iCope responses upon infection and vaccination and the robustness overtime. It was also assessed, if mutations within the iCOPE sequence might significantly impair cross-reactive responses affecting the quality of anti-viral response.

To investigate this the inventors applied an embodiment of the method according to the invention for assessing the functional avidity of T-cells in PBMCs from subjects that were either exposed to SARS-COVID-19 infection, received two consecutive SARS-COVID-19 m-RNA vaccinations or had no SARS-COVID-19 exposure before. This method was employed to test the T-cell avidity in patient samples towards COVID-19 antigens that comprised single nucleic acid mutations in the 15 amino acid SFIEDLLFNKVTLAD (SEQ ID NO 2) of the wild-type “iCOPE” sequence.

Amino Acids S819-826 are Critical for iCope Specific T Cell Responses

The inventors assessed the effect of the different mutations on iCope-specific CD4+ T cell reactivity by comparing 15mer peptide stimulations of wildtype (WT) iCope to the different mutations as well as N-terminal (S-I, aa 1-643) and C-terminal (S-II, aa 633-1273) spike peptide pools and CEFX high control (consisting of a mixture of CMV, EBV, Flu and other peptides). In young, unexposed donors 44% strong (stimulation index (stim. index)>3) and 39% weak responders were observed (stim. index>1.5) (FIG. 12). The mutation F817L causes a drop in 4 more sample donors suggesting a strong HLA-dependent impairment. With exception of the non-conserved N824L position, the stretch 819-826 appears to be critical for effective T cell activation. Also the V826D exchange impaired the T cell activation. In line with the inventors previous findings, where iCope frequencies increased very early after infection until naive; SARS-CoV-2 specific clones are expanded (as indicated by increased levels of S-I and S-II peptide-pool frequencies) and drop close to initial levels after clearance, the frequencies of iCope reactive T cells in convalescents are only slightly higher than in unexposed.

The inventors showed before that the cross-reactive T cell response is dominated by the iCOPE sequence located within the fusion peptide domain (amino acids 816-830). Comparison of the 15mer SARS-CoV-2 spike S816-830 iCope sequence SFIEDLLFNKVTLAD (SEQ ID NO 2) with those of endemic coronaviruses (NL63, 229E, OC43, HKU1) demonstrates high conservation throughout the different coronaviruses. Herein, the inventors observed that mutations, specifically in the central or “core” sequence of the iCOPE peptide comprising at least EDLLFNKV (SEQ ID NO 17) reduced the responsiveness of both COVID-19 unexposed and convalescents, indicating the importance of this central sequence region of the iCOPE peptide for the immune response of subjects, even when the subjects had no exposure to COVID-19 before, but have only been exposed to seasonal endemic coronaviruses.

Vaccination Boosts iCope Immunity in the Younger but not in the Old

The inventors could demonstrate that BNT162b vaccination results in a strong induction of iCope specific T cells. However, aging had a negative impact on the frequencies of spike specific cross-reactive T cells. Therefore, the inventors here assessed the response of iCope specific T cells upon homologous BNT162b vaccination in young (age<40, mean 30.8 years; FIG. 12. E, F) and elderly (age>60, mean 76.5 years) donors. While the impact of mutations remained comparable in both groups, the overall frequencies of responsive T cells were much lower in the elderly (FIG. 12G, H). As already observed in the unvaccinated subjects, the mutations, especially in the central or “core” sequence of the iCOPE peptide comprising at least EDLLFNKV (SEQ ID NO 17), reduced the responsiveness of the T cells of vaccinated subjects to the iCOPE antigen. These effects were less pronounced in in the amino acids flanking the sequence EDLLFNKV (SEQ ID NO 17). These observations indicate the high importance of the central sequence region of the iCOPE peptide for the immune response of subjects, wherein mutations in the outer regions of the 15 amino acid sequence appeared to be less important for the immune reaction and might tolerate certain sequence variations, as it can be derived from the FIGS. 12 to 14.

Age and Mutations Affect the Quality of iCope Specific T Cell Response

Next, the inventors assessed if not only the frequencies but also the quality in terms of TCR affinity is affected by the different mutations and by aging. The inventors showed that CD3 downregulation among the antigen specific CD40L⁺4-1BB+ T cells, is a correlate of TCR affinity. Consistent with the results in unvaccinated subjects, the inventors observed in individuals that received two vaccinations with an mRNA vaccine (BNT/BNT), which encoded the COVID-19 spike protein, a significantly reduced CD3 downregulation when the positions 819-823 and 825 were mutated (FIG. 13). In general, mutations with reduced T cell binding also displayed in lower TCR affinity. A SARS-CoV-2 infection results in the generation of high affine T cell clones targeting S-I and S-II peptides however no substantial change in the iCope-Sequence can be observed after the infection has been cleared. Homologous BNT/BNT vaccination results in the expansion of high affine iCope clones in the young, but not in the elderly (FIG. 13C-D). The remaining iCope specific T cells in the vaccinated elderly moreover display reduced IFNg-driven antiviral responses, especially when mutations were introduced into the central EDLLFNKV (SEQ ID NO 17) iCOPE sequence (FIG. 13D). Mutations in the iCope sequence also resulted in less optimal cytokine responses by the remaining responsive T cells, wherein mutations in the centre of the iCOPE sequence had a greater negative effect on the cytokine response, than sequence variations in the outer amino acids of the 15 amino acid iCOPE peptide (FIG. 14).

### Materials and Methods

### Study Subjects

This study was approved by the Institutional Review board of the Charité (EA/152/20) and written informed consent was obtained from all participants included (38). Participants who tested positive for SARS-CoV-2 RNA in nasopharyngeal swabs by RT-qPCR were classified as convalescent donors. All donors were assessed for age, gender, BMI, comorbidities and medications. Convalescent donors were subclassified according to their symptoms into WHO severity grades. Day of infection was set as day −3 prior to reported symptom onset. Measurement day post symptom onset is indicated in the graphs. Study participants who reported symptoms typical for a SARS-CoV-2 infection were RT-qPCR tested for virus RNA and positive donors were enrolled for follow-up measurements.

### SARS-CoV-2 RT-qPCR

RNA was extracted by using the MagNA Pure 96 system (Roche, Germany). The viral RNA extraction was performed using from 200 μl swab dilution (Swab suspended in 4.3 ml cobas PCR Media, Roche), eluted in 100 μl. SARS-CoV-2 detection was based on two genomic targets (E- and N gene, TIB Molbiol, Berlin, Germany) using 5 μl of the RNA eluate. Quantification of SARS-CoV-2 was done applying calibration curves and using serial diluted photometrically quantified in-vitro transcribed RNA as described before (39). All RT-qPCR's were performed using a LightCycler 480 II (Roche).

### Blood and Serum Sampling and PBMC Isolation

Whole blood was collected in lithium heparin tubes for peripheral blood mononuclear cells (PBMC) isolation and SSTTMII advance (all Vacutainer®, BD) tubes for serology. SSTTMII advance tubes were centrifuged at 1000 g, 10 min and serum supernatant aliquots frozen at −20° C. until further use. PBMCs were isolated by gradient density centrifugation according to the manufacturer's instructions (Leucosep tubes, Greiner; Biocoll, Bio&SELL).

### Ex Vivo T Cell Stimulation

Freshly isolated PBMC were cultivated at 5×10⁶ PBMC in AB-medium containing RPMI 1640 medium (Gibco) supplemented with 10% heat inactivated AB serum (Pan Biotech), 100 U/ml penicillin (Biochrom), 0.1 mg/ml streptomycin (Biochrom). Stimulations were conducted with 11aa overlapping 15-mer PepMix™ SARS-CoV-2, HCoV-229E, HCoV-OC43, HCoV-NL63, HCoV-HKU1 spike glycoprotein peptide pool 1 or 2 (all from JPT) at concentrations of 1 μg/ml per peptide respectively. SARS-CoV-2 orfeome stimulations were performed with NCAP-1, VEMP-1, VME-1, AP3A (ORF3a), NS6, NS7A, NS7B, NS8, ORF9B, ORF10, Y14 (ORF9c), and the ORF1a/b pools: NSP01, NSP02, NSP03a, NSP03b, NSP04, NSP05, NSP06, NSP07, NSP08, NSP09, NSP10, NSP11, NSP12, NSP13, NSP14, NSP15, NSP16 (all from JPT) at concentrations of 1 μg/ml per peptide respectively. Used control peptide pools were hepatitis B virus protein LEP (HBV-LEP), adenovirus penton protein (HAdV5), influenza A Brisbane H1N1 (INFA-HABris), vaccinia virus (VACV), autoantigen actin, human cytomegalovirus protein pp65 (HCMVA), all from JPT. Single peptide stimulation was conducted with 1 μg/ml of the following peptides: 204 (N′-SKRSFIEDLLFNKVT-C′) (SEQ ID NO 9), 204_1 (N′-KRSFIEDLLFNKVTL-C′) (SEQ ID NO 20), 204_2 (N′-RSFIEDLLFNKVTLA-C′) (SEQ ID NO 21), 204_3 (N′-SFIEDLLFNKVTLAD-C′) (SEQ ID NO 2), 205 (N′-FIEDLLFNKVTLADA-C′) (SEQ ID NO 10) or the control peptide 284 (N′-VNNTVYDPLQPELDS-C′) (SEQ ID NO 22) (all from JPT). Stimulation controls were performed with equal concentrations of DMSO in PBS (unstimulated control) and 1.5 mg SEB/1.0 mg TSST1 (Sigma-Aldrich) or 1 μg/ml per peptide of CEFX Ultra SuperStim pool (JPT) (positive control). All approaches contained 1 μg/ml purified anti-CD28 (clone CD28.2, BD Biosciences). Incubation was performed at 37° C., 5% CO2 for 16 h in the presence of 10 μg/ml brefeldin A (Sigma-Aldrich) during the last 14 h. CD4⁺ T cell activation was plotted as Stimulation Index (Stim. index)=% of CD40L⁺4-1BB⁺ CD4⁺ T cells in stimulation/% of CD40L⁺4-1BB⁺ CD4⁺ T cells in unstimulated control. Dotted lines indicate SI of 1.5 (positive with uncertainty) and 3 (certainly positive). For CD3^{low} plots the threshold line is set at 20%.

### T Cell Enrichment and Expansion

Activated cells were enriched from stimulated PBMCs by magnetic cell sorting (MACS). Cells were stimulated with indicated PepMixes in the presence of 1 μg/ml purified anti-CD28 (clone CD28.2, BD Biosciences) and 1 μg/ml purified anti-CD40 (5C3, Biolegend) for 16 h followed by staining with CD40L-APC (5C8, Miltenyi) and 4-1BB-PE (4B4-1, BD). The activated cells were enriched with anti-PE MultiSort MicroBeads (Miltenyi) according to manufacturer's instructions. After release of anti-PE beads a second enrichment was performed with anti-APC MicroBeads (Miltenyi). Purity was routinely checked to >80% of alive cells. Feeder cells were obtained from the 4-1BB-PE negative fraction by CD3 MicroBead (Miltenyi) depletion and subsequent irradiation at 50 Gy. Enriched CD40L⁺4-1BB⁺ cells were co-cultured with feeder cells at a ratio of 1:1 in AB-medium supplemented with 10 ng/ml IL-7 and IL-15 respectively (both from Miltenyi) for 10 d followed by 2 d cytokine starvation. Restimulation in the presence of CD3 depleted, autologous APC was conducted as described above and indicated in the figure legends. For spike glycoprotein epitope identification restimulation was performed with the Epitope Mapping Peptide Set SARS-CoV-2 (JPT) according to the manufacturer's instructions.

### Flow Cytometry

Stimulations were stopped by incubation in 20 mM EDTA for 5 min. Surface staining was performed for 15 min in the presence of 1 mg/ml Beriglobin (CSL Behring) with the following fluorochrome-conjugated antibodies titrated to their optimal concentrations: CD3-FITC (REA613, Miltenyi), CD4-VioGreen (REA623, Miltenyi), CD8-VioBlue (REA734, Miltenyi), CD38-APC (REA671, Miltenyi), HLA-DR-PerCpVio700 (REA805, Miltenyi). During the last 10 min of incubation, Zombie Yellow fixable viability staining (Biolegend) was added. Fixation and permeabilization were performed with eBioscience™ FoxP3 fixation and PermBuffer (Invitrogen) according to the manufacturer's protocol. Intracellular staining was carried out for 30 min in the dark at room temperature with 4-1BB-PE (REA765, Miltenyi) and CD40L-PeVio770 (REA238, Miltenyi). All samples were measured on a MACSQuant® Analyzer 16 (Miltenyi). Instrument performance was monitored prior to every measurement with Rainbow Calibration Particles (BD).

### SARS-CoV-2 IgG S1 ELISA

Anti-SARS-CoV-2 IgG and IgA ELISA specific for S subunit 1 (S1) using a commercial kit (EUROIMMUN Medizinische Labordiagnostika AG) were performed according to the manufacturer's instructions and described previously (40). Maximum values were set as 12 IgA.

### SARS-CoV-2 Neutralization Assay

Neutralization activity of SARS-CoV-2 specific antibodies was assessed with a plaque reduction neutralization test (PRNT) as described before (39). Vero E6 cells (1.6×10⁵ cells/well) were seeded in 24-well plates overnight. Sera were diluted in OptiPro and mixed 1:1 with 200 μl virus (Munich isolate 984) solution containing 100 plaque forming units and incubated at 37° C. for 1 hour. Plated Vero cells were incubated with 200 μl serum-virus solution. After 1 hour at 37° C., the supernatants were discarded and cells were washed once with PBS and overlaid with 1.2% Avicel solution in DMEM. After 3 days at 37° C., the supernatants were removed, the cells were fixed with 6% formaldehyde in PBS and stained with crystal violet. All dilutions were tested in duplicates.

Epitope Specific Antibody ELISA 400 nM of biotinylated peptide 204_-1 (Biotin-Ttds-PSKPSKRSFIEDLLFNKV-OH (SEQ ID NO 19), overlapping with 204_3 by 11 aa (cursive letters), Ttds linker=N-(3-{2-[2-(3-Amino-propoxy)-ethoxy]-ethoxy}-propyl)-succinamic acid, JPT Peptide Technologies) was coated on a 96 well Streptavidin plate (Steffens Biotechnische Analysen GmbH) for 1 h at RT. After blocking (1 h, 30° C.) serum samples were diluted 1:100 and incubated for 1 h at 30° C. HRP-coupled, anti-human-IgG secondary antibody (Jackson Immunoresearch) was diluted 1:5000 (Jackson Immunoresearch) and added to the serum samples for 1 h at 30° C., then HRP substrate was added (TMB, Kem-En-Tec). The reaction was stopped by adding sulfuric acid and absorption was measured at 450 nm using a FlexStation 3.

### HLA Typing and Analysis

HLA typing has been performed by LABType® CWD assays (One Lambda, West Hills, CA, USA) based on reverse sequence-specific oligonucleotides (rSSO) according to the manufacturer's instructions. Briefly, the HLA genomic region has been amplified individually using locus-specific biotinylated primers for HLA-DRB1, -DQA1, -DQB1, -DPA1 and -DPB1. Amplicons were hybridized to HLA allele- and allele-group-specific probes attached to Luminex® beads. Complementary binding was detected by addition of R-phycoerythrin-conjugated streptavidin and acquired using a FLEXMAP 3D flow analyzer (Luminex, Austin, TX, USA). HLA typings were derived as 2-field typing results with the highest probability as referenced in the catalogue of common and well-documented HLA alleles version 2.0.0 33. IEDB Analysis Resource (www.IEDB.org (41, 42), 2.22 method) was used for MHC class II binding prediction of the peptide 2043 and homologous HCoV epitopes. For the purpose of this analysis, we refer to an individual as ‘homozygous’ if the two corresponding alleles of the same locus are identical in the first two fields.

### Homology Score

For the calculation of the homology score all possible 9mers were generated for each respective PepMix of SARS-CoV-2. Each of the 9mers was scored against each unique 9mer from the proteomes of the corona viruses 229E, NL63, OC43 and HKU1 (isolates N1, N2, N5) using the PAM30 substitution matrix. The homology score is the percentage of comparisons with a pairwise 9mer-score above 30.

### Data Analysis and Statistics

Study data were collected and managed using REDCap electronic data capture tools hosted at Charité (43, 44). Flow cytometry data were analyzed with FlowJo 10.6 (FlowJo LLC) and statistical analysis conducted with GraphPad Prism 9. If not stated otherwise, data are plotted as mean. N indicates the number of donors. P-values were set as follows: *p<0.05, ** p<0.01, and *** p<0.001.

### REFERENCES

1. E. J. Williamson, A. J. Walker, K. Bhaskaran, S. Bacon, C. Bates, C. E. Morton, H. J. Curtis, A. Mehrkar, D. Evans, P. Inglesby, J. Cockburn, H. I. McDonald, B. MacKenna, L. Tomlinson, I. J. Douglas, C. T. Rentsch, R. Mathur, A. Y. S. Wong, R. Grieve, D. Harrison, H. Forbes, A. Schultze, R. Croker, J. Parry, F. Hester, S. Harper, R. Perera, S. J. W. Evans, L. Smeeth, B. Goldacre, Factors associated with COVID-19-related death using OpenSAFELY. Nature. 584, 430-436 (2020).
2. U. Stervbo, S. Rahmann, T. Roch, T. H. Westhoff, N. Babel, Epitope similarity cannot explain the pre-formed T cell immunity towards structural SARS-CoV-2 proteins. Scientific Reports. 10, 18995 (2020).
3. J. Braun, L. Loyal, M. Frentsch, D. Wendisch, P. Georg, F. Kurth, S. Hippenstiel, M. Dingeldey, B. Kruse, F. Fauchere, E. Baysal, M. Mangold, L. Henze, R. Lauster, M. A. Mall, K. Beyer, J. Röhmel, S. Voigt, J. Schmitz, S. Miltenyi, I. Demuth, M. A. Müller, A. Hocke, M. Witzenrath, N. Suttorp, F. Kern, U. Reimer, H. Wenschuh, C. Drosten, V. M. Corman, C. Giesecke-Thiel, L. E. Sander, A. Thiel, SARS-CoV-2-reactive T cells in healthy donors and patients with COVID-19. Nature. 587, 270-274 (2020).
4. A. Grifoni, D. Weiskopf, S. I. Ramirez, J. Mateus, J. M. Dan, C. R. Moderbacher, S. A. Rawlings, A. Sutherland, L. Premkumar, R. S. Jadi, D. Marrama, A. M. de Silva, A. Frazier, A. F. Carlin, J. A. Greenbaum, B. Peters, F. Krammer, D. M. Smith, S. Crotty, A. Sette, Targets of T Cell Responses to SARS-CoV-2 Coronavirus in Humans with COVID-19 Disease and Unexposed Individuals. Gell. 181, 1489-1501.e15 (2020).
5. A. Nelde, T. Bilich, J. S. Heitmann, Y. Maringer, H. R. Salih, M. Roerden, M. Lübke, J. Bauer, J. Rieth, M. Wacker, A. Peter, S. Hörber, B. Traenkle, P. D. Kaiser, U. Rothbauer, M. Becker, D. Junker, G. Krause, M. Strengert, N. Schneiderhan-Marra, M. F. Templin, T. O. Joos, D. J. Kowalewski, V. Stos-Zweifel, M. Fehr, A. Rabsteyn, V. Mirakaj, J. Karbach, E. Jäger, M. Graf, L.-C. Gruber, D. Rachfalski, B. Preuß, I. Hagelstein, M. Märklin, T. Bakchoul, C. Gouttefangeas, O. Kohlbacher, R. Klein, S. Stevanović, H.-G. Rammensee, J. S. Walz, SARS-CoV-2-derived peptides define heterologous and COVID-19-induced T cell recognition. Nature Immunology. 22, 74-85 (2021).
6. N. Le Bert, A. T. Tan, K. Kunasegaran, C. Y. L. Tham, M. Hafezi, A. Chia, M. H. Y. Chng, M. Lin, N. Tan, M. Linster, W. N. Chia, M. I.-C. Chen, L.-F. Wang, E. E. Ooi, S. Kalimuddin, P. A. Tambyah, J. G.-H. Low, Y.-J. Tan, A. Bertoletti, SARS-CoV-2-specific T cell immunity in cases of COVID-19 and SARS, and uninfected controls. Nature. 584, 457-462 (2020).
7. J. Mateus, A. Grifoni, A. Tarke, J. Sidney, S. I. Ramirez, J. M. Dan, Z. C. Burger, S. A. Rawlings, D. M. Smith, E. Phillips, S. Mallal, M. Lammers, P. Rubiro, L. Quiambao, A. Sutherland, E. D. Yu, R. da S. Antunes, J. Greenbaum, A. Frazier, A. J. Markmann, L. Premkumar, A. de Silva, B. Peters, S. Crotty, A. Sette, D. Weiskopf, Selective and cross-reactive SARS-CoV-2 T cell epitopes in unexposed humans. Science. 370, 89-94 (2020).
8. P. Bacher, E. Rosati, D. Esser, G. R. Martini, C. Saggau, E. Schiminsky, J. Dargvainiene, I. Schröder, I. Wieters, Y. Khodamoradi, F. Eberhardt, M. J. G. T. Vehreschild, H. Neb, M. Sonntagbauer, C. Conrad, F. Tran, P. Rosenstiel, R. Markewitz, K.-P. Wandinger, M. Augustin, J. Rybniker, M. Kochanek, F. Leypoldt, O. A. Comely, P. Koehler, A. Franke, A. Scheffold, Low-Avidity CD4+ T Cell Responses to SARS-CoV-2 in Unexposed Individuals and Humans with Severe COVID-19. Immunity. 53, 1258-1271.e5 (2020).
9. E. M. Anderson, E. C. Goodwin, A. Verma, C. P. Arevalo, M. J. Bolton, M. E. Weirick, S. Gouma, C. M. McAllister, S. R. Christensen, J. Weaver, P. Hicks, T. B. Manzoni, O. Oniyide, H. Ramage, D. Mathew, A. E. Baxter, D. A. Oldridge, A. R. Greenplate, J. E. Wu, C. Alanio, K. D'Andrea, O. Kuthuru, J. Dougherty, A. Pattekar, J. Kim, N. Han, S. A. Apostolidis, A. C. Huang, L. A. Vella, L. Kuri-Cervantes, M. B. Pampena, M. R. Betts, E. J. Wherry, N. J. Meyer, S. Cherry, P. Bates, D. J. Rader, S. E. Hensley, Seasonal human coronavirus antibodies are boosted upon SARS-CoV-2 infection but not associated with protection. Gell. 0 (2021), doi:10.1016/j.cell.2021.02.010.
10. N. Mishra, X. Huang, S. Joshi, C. Guo, J. Ng, R. Thakkar, Y. Wu, X. Dong, Q. Li, R. S. Pinapati, E. Sullivan, A. Caciula, R. Tokarz, T. Briese, J. Lu, W. I. Lipkin, Immunoreactive peptide maps of SARS-CoV-2. Gommunications Biology. 4, 1-7 (2021).
11. A. P. Ferretti, T. Kula, Y. Wang, D. M. V. Nguyen, A. Weinheimer, G. S. Dunlap, Q. Xu, N. Nabilsi, C. R. Perullo, A. W. Cristofaro, H. J. Whitton, A. Virbasius, K. J. Olivier, L. R. Buckner, A. T. Alistar, E. D. Whitman, S. A. Bertino, S. Chattopadhyay, G. MacBeath, Unbiased Screens Show CD8+ T Cells of COVID-19 Patients Recognize Shared Epitopes in SARS-CoV-2 that Largely Reside outside the Spike Protein. Immunity. 53, 1095-1107.e3 (2020).
12. K. W. Ng, N. Faulkner, G. H. Cornish, A. Rosa, R. Harvey, S. Hussain, R. Ulferts, C. Earl, A. G. Wrobel, D. J. Benton, C. Roustan, W. Bolland, R. Thompson, A. Agua-Doce, P. Hobson, J. Heaney, H. Rickman, S. Paraskevopoulou, C. F. Houlihan, K. Thomson, E. Sanchez, G. Y. Shin, M. J. Spyer, D. Joshi, N. O'Reilly, P. A. Walker, S. Kjaer, A. Riddell, C. Moore, B. R. Jebson, M. Wilkinson, L. R. Marshall, E. C. Rosser, A. Radziszewska, H. Peckham, C. Ciurtin, L. R. Wedderburn, R. Beale, C. Swanton, S. Gandhi, B. Stockinger, J. McCauley, S. J. Gamblin, L. E. McCoy, P. Cherepanov, E. Nastouli, G. Kassiotis, Preexisting and de novo humoral immunity to SARS-CoV-2 in humans. Science. 370, 1339-1343 (2020).
13. D. Weiskopf, K. S. Schmitz, M. P. Raadsen, A. Grifoni, N. M. A. Okba, H. Endeman, J. P. C. van den Akker, R. Molenkamp, M. P. G. Koopmans, E. C. M. van Gorp, B. L. Haagmans, R. L. de Swart, A. Sette, R. D. de Vries, Phenotype and kinetics of SARS-CoV-2-specific T cells in COVID-19 patients with acute respiratory distress syndrome. Science Immunology. 5 (2020), doi:10.1126/sciimmunol.abd2071.
14. T. Sekine, A. Perez-Potti, O. Rivera-Ballesteros, K. Stralin, J.-B. Gorin, A. Olsson, S. Llewellyn-Lacey, H. Kamal, G. Bogdanovic, S. Muschiol, D. J. Wullimann, T. Kammann, J. Emgard, T. Parrot, E. Folkesson, Karolinska COVID-19 Study Group, O. Rooyackers, L. I. Eriksson, J.-O. Henter, A. Sönnerborg, T. Allander, J. Albert, M. Nielsen, J. Klingström, S. Gredmark-Russ, N. K. Björkström, J. K. Sandberg, D. A. Price, H.-G. Ljunggren, S. Aleman, M. Buggert, Robust T Cell Immunity in Convalescent Individuals with Asymptomatic or Mild COVID-19. Gell. 183, 158-168.e14 (2020).
15. A. A. Lehmann, G. A. Kirchenbaum, T. Zhang, P. A. Reche, P. V. Lehmann, bioRxiv, in press, doi:10.1101/2020.11.29.402677.
16. W. S. Lee, A. K. Wheatley, S. J. Kent, B. J. DeKosky, Antibody-dependent enhancement and SARS-CoV-2 vaccines and therapies. Nature Microbiology. 5, 1185-1191 (2020).
17. M. Sagar, K. Reifler, M. Rossi, N. S. Miller, P. Sinha, L. F. White, J. P. Mizgerd, Recent endemic coronavirus infection is associated with less-severe COVID-19. J Glin Invest. 131 (2021), doi:10.1172/JCI143380.
18. L. Liu, P. Wang, M. S. Nair, J. Yu, M. Rapp, Q. Wang, Y. Luo, J. F.-W. Chan, V. Sahi, A. Figueroa, X. V. Guo, G. Cerutti, J. Bimela, J. Gorman, T. Zhou, Z. Chen, K.-Y. Yuen, P. D. Kwong, J. G. Sodroski, M. T. Yin, Z. Sheng, Y. Huang, L. Shapiro, D. D. Ho, Potent neutralizing antibodies against multiple epitopes on SARS-CoV-2 spike. Nature. 584, 450-456 (2020).
19. D. L. Farber, N. A. Yudanin, N. P. Restifo, Human memory T cells: generation, compartmentalization and homeostasis. Nature Reviews Immunology. 14, 24-35 (2014).
20. H. Liu, M. Rhodes, D. L. Wiest, D. A. A. Vignali, On the Dynamics of TCR:CD3 Complex Cell Surface Expression and Downmodulation. Immunity. 13, 665-675 (2000).
21. P. Holenya, P. J. Lange, U. Reimer, W. Woltersdorf, T. Panterodt, M. Glas, M. Wasner, M. Eckey, M. Drosch, J.-M. Hollidt, M. Naumann, F. Kern, H. Wenschuh, R. Lange, K. Schnatbaum, F. F. Bier, medRxiv, in press, doi:10.1101/2020.11.24.20216663.
22. A. A. Minervina, M. V. Pogorelyy, E. A. Komech, V. K. Karnaukhov, P. Bacher, E. Rosati, A. Franke, D. M. Chudakov, I. Z. Mamedov, Y. B. Lebedev, T. Mora, A. M. Walczak, Primary and secondary anti-viral response captured by the dynamics and phenotype of individual T cell clones. eLife. 9, e53704 (2020).
23. K. Fink, Origin and Function of Circulating Plasmablasts during Acute Viral Infections. Front. Immunol. 3 (2012), doi:10.3389/fimmu.2012.00078.
24. D. Poston, Y. Weisblum, H. Wise, K. Templeton, S. Jenks, T. Hatziioannou, P. Bieniasz, Absence of Severe Acute Respiratory Syndrome Coronavirus 2 Neutralizing Activity in Prepandemic Sera From Individuals With Recent Seasonal Coronavirus Infection. Clinical Infectious Diseases (2020), doi:10.1093/cid/ciaa1803.
25. W. R. Morgenlander, S. N. Henson, D. R. Monaco, A. Chen, K. Littlefield, E. M. Bloch, E. Fujimura, I. Ruczinski, A. R. Crowley, H. Natarajan, S. E. Butler, J. A. Weiner, M. Z. Li, T. S. Bonny, S. E. Benner, A. Balagopal, D. Sullivan, S. Shoham, T. C. Quinn, S. Eshleman, A. Casadevall, A. D. Redd, O. Laeyendecker, M. E. Ackerman, A. Pekosz, S. J. Elledge, M. L. Robinson, A. A. R. Tobian, H. B. Larman, Antibody responses to endemic coronaviruses modulate COVID-19 convalescent plasma functionality. J Clin Invest (2021), doi:10.1172/JC1146927.
26. A. Tarke, J. Sidney, C. K. Kidd, J. M. Dan, S. I. Ramirez, E. D. Yu, J. Mateus, R. da Silva Antunes, E. Moore, P. Rubiro, N. Methot, E. Phillips, S. Mallal, A. Frazier, S. A. Rawlings, J. A. Greenbaum, B. Peters, D. M. Smith, S. Crotty, D. Weiskopf, A. Grifoni, A. Sette, Comprehensive analysis of T cell immunodominance and immunoprevalence of SARS-CoV-2 epitopes in COVID-19 cases. Cell Rep Med. 2, 100204 (2021).
27. T. A. Schwickert, B. Alabyev, T. Manser, M. C. Nussenzweig, Germinal center reutilization by newly activated B cells. Journal of Experimental Medicine. 206, 2907-2914 (2009).
28. T. A. Schwickert, G. D. Victora, D. R. Fooksman, A. O. Kamphorst, M. R. Mugnier, A. D. Gitlin, M. L. Dustin, M. C. Nussenzweig, A dynamic T cell-limited checkpoint regulates affinity-dependent B cell entry into the germinal center. Journal of Experimental Medicine. 208, 1243-1252 (2011).
29. K. Fink, N. Manjarrez-Orduño, A. Schildknecht, J. Weber, B. M. Senn, R. M. Zinkernagel, H. Hengartner, B Cell Activation State-Governed Formation of Germinal Centers following Viral Infection. The Journal of Immunology. 179, 5877-5885 (2007).
30. A. T. Tan, M. Linster, C. W. Tan, N. L. Bert, W. N. Chia, K. Kunasegaran, Y. Zhuang, C. Y. L. Tham, A. Chia, G. J. D. Smith, B. Young, S. Kalimuddin, J. G. H. Low, D. Lye, L.-F. Wang, A. Bertoletti, Early induction of functional SARS-CoV-2-specific T cells associates with rapid viral clearance and mild disease in COVID-19 patients. Cell Reports. 34 (2021), doi:10.1016/j.celrep.2021.108728.
31. M. Lipsitch, Y. H. Grad, A. Sette, S. Crotty, Cross-reactive memory T cells and herd immunity to SARS-CoV-2. Nature Reviews Immunology. 20, 709-713 (2020).
32. A. T. Huang, B. Garcia-Carreras, M. D. T. Hitchings, B. Yang, L. C. Katzelnick, S. M. Rattigan, B. A. Borgert, C. A. Moreno, B. D. Solomon, L. Trimmer-Smith, V. Etienne, I. Rodriguez-Barraquer, J. Lessler, H. Salje, D. S. Burke, A. Wesolowski, D. A. T. Cummings, A systematic review of antibody mediated immunity to coronaviruses: kinetics, correlates of protection, and association with severity. Nature Communications. 11, 4704 (2020).
33. S. Amit, G. Regev-Yochay, A. Afek, Y. Kreiss, E. Leshem, Early rate reductions of SARS-CoV-2 infection and COVID-19 in BNT162b2 vaccine recipients. The Lancet. 0 (2021), doi:10.1016/S0140-6736(21)00448-7.
34. J. L. Bernal, N. Andrews, C. Gower, J. Stowe, C. Robertson, E. Tessier, R. Simmons, S. Cottrell, R. Roberts, M. O'Doherty, K. Brown, C. Cameron, D. Stockton, J. McMenamin, M. Ramsay, medRxiv, in press, doi:10.1101/2021.03.01.21252652.
35. C. M. Saad-Roy, S. E. Morris, C. J. E. Metcalf, M. J. Mina, R. E. Baker, J. Farrar, E. C. Holmes, O. G. Pybus, A. L. Graham, S. A. Levin, B. T. Grenfell, C. E. Wagner, Epidemiological and evolutionary considerations of SARS-CoV-2 vaccine dosing regimes. Science (2021), doi:10.1126/science.abg8663.
36. A. L. Lai, J. K. Millet, S. Daniel, J. H. Freed, G. R. Whittaker, The SARS-CoV Fusion Peptide Forms an Extended Bipartite Fusion Platform that Perturbs Membrane Order in a Calcium-Dependent Manner. Journal of Molecular Biology. 429, 3875-3892 (2017).
37. S. Ravichandran, Y. Lee, G. Grubbs, E. M. Coyle, L. Klenow, O. Akasaka, M. Koga, E. Adachi, M. Saito, I. Nakachi, T. Ogura, R. Baba, M. Ito, M. Kiso, A. Yasuhara, S. Yamada, Y. Sakai-Tagawa, K. Iwatsuki-Horimoto, M. Imai, S. Yamayoshi, H. Yotsuyanagi, Y. Kawaoka, S. Khurana, Longitudinal antibody repertoire in “mild” versus “severe” COVID-19 patients reveals immune markers associated with disease severity and resolution. Science Advances. 7, eabf2467 (2021).
38. F. Kurth, M. Roennefarth, C. Thibeault, V. M. Corman, H. Müller-Redetzky, M. Mittermaier, C. Ruwwe-Glösenkamp, K. M. Heim, A. Krannich, S. Zvorc, S. Schmidt, L. Kretzler, C. Dang-Heine, M. Rose, M. Hummel, A. Hocke, R. H. Hübner, B. Opitz, M. A. Mall, J. Röhmel, U. Landmesser, B. Pieske, S. Knauss, M. Endres, J. Spranger, F. P. Mockenhaupt, F. Tacke, S. Treskatsch, S. Angermair, B. Siegmund, C. Spies, S. Weber-Carstens, K.-U. Eckardt, D. Schürmann, A. Uhrig, M. S. Stegemann, T. Zoller, C. Drosten, N. Suttorp, M. Witzenrath, S. Hippenstiel, C. von Kalle, L. E. Sander, Studying the pathophysiology of coronavirus disease 2019: a protocol for the Berlin prospective COVID-19 patient cohort (Pa-COVID-19). Infection. 48, 619-626 (2020).
39. R. Wölfel, V. M. Corman, W. Guggemos, M. Seilmaier, S. Zange, M. A. Müller, D. Niemeyer, T. C. Jones, P. Vollmar, C. Rothe, M. Hoelscher, T. Bleicker, S. Brünink, J. Schneider, R. Ehmann, K. Zwirglmaier, C. Drosten, C. Wendtner, Virological assessment of hospitalized patients with COVID-2019. Nature, 1-10 (2020).
40. N. M. A. Okba, M. A. Muller, W. Li, C. Wang, C. H. GeurtsvanKessel, V. M. Corman, M. M. Lamers, R. S. Sikkema, E. de Bruin, F. D. Chandler, Y. Yazdanpanah, Q. L. Hingrat, D. Descamps, N. Houhou-Fidouh, C. B. E. M. Reusken, B.-J. Bosch, C. Drosten, M. P. G. Koopmans, B. L. Haagmans, medRxiv, in press, doi:10.1101/2020.03.18.20038059.
41. P. Wang, J. Sidney, C. Dow, B. Mothé, A. Sette, B. Peters, A Systematic Assessment of MHC Class II Peptide Binding Predictions and Evaluation of a Consensus Approach. PLOS Computational Biology. 4, e1000048 (2008).
42. P. Wang, J. Sidney, Y. Kim, A. Sette, O. Lund, M. Nielsen, B. Peters, Peptide binding predictions for HLA DR, DP and DQ molecules. BMC Bioinformatics. 11, 568 (2010).
43. P. A. Harris, R. Taylor, B. L. Minor, V. Elliott, M. Fernandez, L. O'Neal, L. McLeod, G. Delacqua, F. Delacqua, J. Kirby, S. N. Duda, The REDCap consortium: Building an international community of software platform partners. Journal of Biomedical Informatics. 95, 103208 (2019).
44. P. A. Harris, R. Taylor, R. Thielke, J. Payne, N. Gonzalez, J. G. Conde, Research electronic data capture (REDCap)—A metadata-driven methodology and workflow process for providing translational research informatics support. Journal of Biomedical Informatics. 42, 377-381 (2009).

1. A method for assessing the functional avidity of T cells for one or more antigens, the method comprising:
providing a cell population comprising T cells,
contacting said cell population with one or more antigens in vitro, and
determining a level of a T-cell receptor (TCR) complex and/or component thereof in a sub-set of T cells of said cell population.

2. The method according to claim 1, wherein the cell population comprising a T cell comprises, or is derived from, a bodily fluid from a subject.

3. The method according to any one or more of the preceding claims, wherein the bodily fluid from a subject is blood or part thereof, or cerebrospinal fluid.

4. The method according to any one or more of the preceding claims, wherein the cell population comprising T cells is an in vitro cell population of T cells, for example a population of T cells isolated from a subject and cultivated in vitro, or a population of T cells generated in vitro.

5. The method according to any one or more of the preceding claims, wherein the cell population comprising T cells comprises peripheral blood mononuclear cells (PBMC).

6. The method according to any one or more of the preceding claims, wherein the sub-set of cells in which a T-cell receptor (TCR) and/or component thereof is determined comprises T helper cells.

7. The method according to any one or more of the preceding claims, wherein the sub-set of cells in which a T-cell receptor (TCR) and/or component thereof is determined comprises CD4+ T cells.

8. The method according to any one or more of the preceding claims, wherein the sub-set of cells in which a T-cell receptor (TCR) and/or component thereof is determined comprises activated T helper cells, preferably CD40L+, 4-1BB+, CD8+ and/or CD4+ T cells.

9. The method according to any one or more of the preceding claims, wherein determining a level of a T-cell receptor (TCR) complex and/or component thereof comprises determining a level of TCR alpha (α), beta (β), gamma (γ) or delta (δ) chains, preferably constant regions thereof, or cluster of differentiation 3 (CD3).

10. The method according to claim 9, wherein determining a level of a T-cell receptor (TCR) complex and/or component thereof comprises determining a level of CD3.

11. The method according to any one or more of the preceding claims, comprising determining the proportion of cells in said sub-set of cells in which the level of a T-cell receptor (TCR) complex and/or component thereof is below a first threshold value (TCR^{low} cells).

12. The method according to the preceding claim, comprising determining the proportion of cells in said sub-set of cells in which the level of CD3 is below a first threshold value (CD3^{low} cells).

13. The method according to any one or more of the preceding claims, wherein the proportion of TCR^{low} (preferably CD3^{low}) cells in said sub-set of cells indicates a level of functional T cell avidity for the antigen.

14. The method according to the preceding claim, wherein the proportion of TCR^{low} (preferably CD3^{low}) cells in said sub-set of cells is positively correlated with a level of functional T cell avidity for the antigen.

15. The method according to any one or more of the preceding claims, wherein determining the level of a T-cell receptor (TCR) complex and/or component thereof is carried out using flow cytometry of antibody labelled cells.

16. The method according to any one or more of the preceding claims, wherein determining the level of a T-cell receptor (TCR) complex and/or component thereof in a sub-set of T cells of said cell population is carried out using flow cytometry of antibody labelled cells, wherein the sub-set of cells is labelled using one or more of CD40L, 4-1BB, CD4, CD8 and CD3 antibodies.

17. The method according to any one or more of the preceding claims, wherein contacting said cell population with one or more antigens in vitro comprises cultivation of said cells with said one or more antigens for 2 to 36 hours.

18. The method according to any one or more of the preceding claims, wherein contacting said cell population with one or more antigens in vitro comprises cultivation of said cells with said one or more antigens for 10 to 24 hours, preferably 12 to 20 hours, more preferably about 16 hours.

19. The method according to any one or more of the preceding claims, comprising additionally determining a level of one more cytokines after contacting said cell population with one or more antigens in vitro.

20. The method according to any one or more of the preceding claims, wherein said one or more antigens comprises or consists of one or more ligands, antigens, pathogens and/or mixtures or fragments thereof.

21. The method according to any one of the preceding claims, wherein the method is employed to assess the strength and/or quality of an immune response in a subject, from which said cell population comprising T cells was obtained, against one or more antigens of interest.

22. The method according to any one of the preceding claims, wherein the one or more antigens is of pathological relevance.

23. The method according to any one of the preceding claims, wherein the one or more antigens comprise an autoantigen, a tumor antigen, a pathogen, or antigenic part or mixture thereof.

24. The method according to any one of the preceding claims, wherein the method is employed to monitor the status of a disease and/or condition of a subject with a persistent immune-related medical condition, such as a viral infection, such as HCV, HIV infection, or an autoimmune disease.

25. The method according to any one of the preceding claims, wherein the one or more antigens is, comprises and/or is derived from SARS-CoV, preferably SARS-CoV-2.

26. The method according to the preceding claim, wherein the one or more antigens is, comprises and/or is derived from SARS-CoV-2 spike glycoprotein, preferably SARS-CoV-2 spike glycoprotein S-II.

27. The method according to the preceding claim, wherein the one or more antigens is, a peptide of up to 25 amino acids comprising or consisting of an amino acid sequence FIEDLLFNKVT (SEQ ID NO 1) or a sequence of at least 80%, preferably at least 90%, sequence identity thereto, preferably of 15 to 25 amino acids, more preferably comprising IEDLLFNKV (SEQ ID NO 3) or EDLLFNKVT (SEQ ID NO 4) or FIEDLLFNKVT (SEQ ID NO 1) and optionally 1-4 additional amino acid at the N and/or C-termini.

28. The method according to any one of claims 25-27, wherein the method is employed to assess (preferably prognose) an immune response of said subject to a SARS-CoV (i.e. to assess the strength of an immune response against a SARS-CoV infection), or to assess a risk of a subject in developing a severe acute respiratory syndrome (SARS) or other adverse event or severe medical condition associated with a SARS-CoV (preferably SARS-CoV-2) infection.

29. A kit for assessing the functional avidity of T cells for one or more antigens, comprising:
means for determining a level of a T-cell receptor (TCR) complex and/or component thereof in a sub-set of T cells of a cell population, preferably comprising one or more antibodies, more preferably comprising one or more labelled antibodies, wherein the means preferably detect CD3, and
one or more antigens of pathological relevance,
and optionally, means for providing, maintaining and/or culturing a cell population comprising T cells and contacting said cell population with said one or more antigens in vitro, and
optionally, means for determining a level of one more cytokines, preferably one or more antibodies.


SEQUENCE LISTING (29 sequences)

SEQ ID NO: 1 - protein, 11 aa, Severe acute respiratory syndrome coronavirus 2
FIEDLLFNKVT

SEQ ID NO: 2 - protein, 15 aa, Severe acute respiratory syndrome coronavirus 2
SFIEDLLFNKVTLAD

SEQ ID NO: 3 - protein, 9 aa, Severe acute respiratory syndrome coronavirus 2
IEDLLFNKV

SEQ ID NO: 4 - protein, 9 aa, Severe acute respiratory syndrome coronavirus 2
EDLLFNKVT

SEQ ID NO: 5 - protein, 4 aa, Severe acute respiratory syndrome coronavirus 2
LADA

SEQ ID NO: 6 - protein, 10 aa, Severe acute respiratory syndrome coronavirus 2
FIEDLLFNKV

SEQ ID NO: 7 - protein, 10 aa, Severe acute respiratory syndrome coronavirus 2
IEDLLFNKVT

SEQ ID NO: 8 - protein, 4 aa, Severe acute respiratory syndrome coronavirus 2
SKRS

SEQ ID NO: 9 - protein, 15 aa, Severe acute respiratory syndrome coronavirus 2
SKRSFIEDLLFNKVT

SEQ ID NO: 10 - protein, 15 aa, Severe acute respiratory syndrome coronavirus 2
FIEDLLFNKVTLADA

SEQ ID NO: 11 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1270 aa, Severe acute respiratory syndrome coronavirus 2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1271 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1285 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 16 - protein, 25 aa, Severe acute respiratory syndrome coronavirus 2
SKPSKRSFIEDLLFNKVTLADAGFI

SEQ ID NO: 17 - protein, 8 aa, Severe acute respiratory syndrome coronavirus 2
EDLLFNKV

SEQ ID NO: 18 - protein, 8 aa, Severe acute respiratory syndrome coronavirus 2
SFIEDLLF

SEQ ID NO: 19 - protein, 18 aa, Severe acute respiratory syndrome coronavirus 2
PSKPSKRSFIEDLLFNKV

SEQ ID NO: 20 - protein, 15 aa, Severe acute respiratory syndrome coronavirus 2
KRSFIEDLLFNKVTL

SEQ ID NO: 21 - protein, 15 aa, Severe acute respiratory syndrome coronavirus 2
RSFIEDLLFNKVTLA

SEQ ID NO: 22 - protein, 15 aa, Artificial Sequence
  control peptide
VNNTVYDPLQPELDS

SEQ ID NO: 23 - protein, 84 aa, Coronavirus
KQYTSACKTIEDALRLSAHLETNDVSSMLTFDSNAFSLANVTSFGDYNLSSVLPQRNIRSSRIAGRSALEDLLFSKVVTSGLGT

SEQ ID NO: 24 - protein, 84 aa, Coronavirus
KQYTSACKTIEDALRNSAMLESADVSEMLTFDKKAFTLANVSSFGDYNLSSVIPSLPRSGSRVAGRSAIEDILFSKLVTSGLGT

SEQ ID NO: 25 - protein, 89 aa, Coronavirus
SEYGTFCDNINSILNEVNDLLDITQLQVANALMQGVTLSSNLNTNLHSDVDNIDFKSLLGCLGSQCGSSSRSLLEDLLFNKVKLSDVGF

SEQ ID NO: 26 - protein, 90 aa, Coronavirus
VEYGSFCDNINAILTEVNELLDTTQLQVANSLMNGVTLSTKLKDGVNFNVDDINFAPVLGCLGSECSKASSRSAIEDLLFDKVKLSDVGF

SEQ ID NO: 27 - protein, 84 aa, Coronavirus
REYGQFCSKINQALHGANLRQDDSVRNLFASVKSSQSSPIIPGFGGDFNLTLLEPVSISTGSRSARSAIEDLLFDKVTIADPGY

SEQ ID NO: 28 - protein, 80 aa, Coronavirus
LQYGSFCTQLNRALSGIAAEQDRNTREVFAQVKQMYKTPTLKYFGGFNFSQILPDPLKPTKRSFIEDLLFNKVTLADAGF

SEQ ID NO: 29 - protein, 80 aa, Severe acute respiratory syndrome coronavirus 2
LQYGSFCTQLNRALTGIAVEQDKNTQEVFAQVKQIYKTPPIKDFGGFNFSQILPDPSKPSKRSFIEDLLFNKVTLADAGF